SEQUENCE LISTING (18 sequences)

SEQ ID NO: 1 - DNA, 20 bp, Artificial Sequence
  Description of Artificial Sequence Synthetic primer
gtgtgctaaccgtacctggc

SEQ ID NO: 2 - DNA, 20 bp, Artificial Sequence
  Description of Artificial Sequence Synthetic primer
ctggtgatttccttgaccgg

SEQ ID NO: 3 - DNA, 19 bp, Artificial Sequence
  Description of Artificial Sequence Synthetic primer
ccaaggcaggttcaagagg

SEQ ID NO: 4 - DNA, 21 bp, Artificial Sequence
  Description of Artificial Sequence Synthetic primer
tccagcagaaagagaagagga

SEQ ID NO: 5 - DNA, 19 bp, Artificial Sequence
  Description of Artificial Sequence Synthetic primer
ccatggcagaaggaggagg

SEQ ID NO: 6 - DNA, 20 bp, Artificial Sequence
  Description of Artificial Sequence Synthetic primer
attggatggcactagctgcg

SEQ ID NO: 7 - DNA, 21 bp, Artificial Sequence
  Description of Artificial Sequence Synthetic primer
ctgacttgggtctgggctatg

SEQ ID NO: 8 - DNA, 24 bp, Artificial Sequence
  Description of Artificial Sequence Synthetic primer
ttgtcactcaccagccttctattt

SEQ ID NO: 9 - DNA, 21 bp, Artificial Sequence
  Description of Artificial Sequence Synthetic primer
caacaagcccacagggtatgg

SEQ ID NO: 10 - DNA, 21 bp, Artificial Sequence
  Description of Artificial Sequence Synthetic primer
gcactccctctacttgcgttc

SEQ ID NO: 11 - DNA, 19 bp, Artificial Sequence
  Description of Artificial Sequence Synthetic primer
cttgtaaaatgtgcggaga

SEQ ID NO: 12 - DNA, 20 bp, Artificial Sequence
  Description of Artificial Sequence Synthetic primer
gtctttcaccatgaagcact

SEQ ID NO: 13 - DNA, 20 bp, Artificial Sequence
  Description of Artificial Sequence Synthetic primer
gagacagccaagtcctattg

SEQ ID NO: 14 - DNA, 20 bp, Artificial Sequence
  Description of Artificial Sequence Synthetic primer
ccctcgatgtagtagcagac

SEQ ID NO: 15 - DNA, 20 bp, Artificial Sequence
  Description of Artificial Sequence Synthetic primer
aggaccttgcatactgtctc

SEQ ID NO: 16 - DNA, 20 bp, Artificial Sequence
  Description of Artificial Sequence Synthetic primer
actccttggtagccttcttt

SEQ ID NO: 17 - DNA, 20 bp, Artificial Sequence
  Description of Artificial Sequence Synthetic primer
cgccgctagaggtgaaattc

SEQ ID NO: 18 - DNA, 19 bp, Artificial Sequence
  Description of Artificial Sequence Synthetic primer
ttggcaaatgctttcgctc

(19) United States
(12) Patent Application Publication
(10) Pub. No.: US 2015/0086511 A1
(43) Pub. Date: Mar. 26, 2015

(54) SURVIVAL, DIFFERENTIATION AND STRUCTURAL INTEGRATION OF HUMAN NEURAL STEM CELLS GRAFTED INTO THE ADULT RAT SPINAL CORD

(71) Applicants: NEURALSTEM, INC., Rockville, MD (US); The Johns Hopkins University, Baltimore, MD (US)

(72) Inventors: Vassilis E. Koliatsos, Ruxton, MD (US); Jun Yan, Annandale, VA (US); Karl K. Johe, Potomac, MD (US)

(73) Assignees: NEURALSTEM, INC., Rockville, MD (US); THE JOHNS HOPKINS UNIVERSITY, Baltimore, MD (US)

(21) Appl. No.: 14/169,860

(22) Filed: Jan. 31, 2014

Related U.S. Application Data
(63) Continuation of application No. 11/922,279, filed on Jun. 1, 2009, now abandoned.
(60) Provisional application No. 60/796,434, filed on May 1, 2006.
(60) Provisional application No. 60/690,017, filed on Jun. 13, 2005.
(60) Provisional application No. 60/690,033, filed on Jun. 13, 2005.

(51) Int. Cl. A61K 35/30

(52) U.S. Cl. CPC A61K 35/30
USPC 424/93.7

(57) ABSTRACT

The present invention provides methods and compositions for treating spinal cord diseases and injuires. The methods involve transplanting neural stem cells which have been previously expanded in vitro into a patient such that the cells can ameliorate the disease or injury. The stem cells to be transplanted are derived from spinal cord tissue.

### CROSS REFERENCE TO RELATED APPLICATIONS

This application is a continuation of U.S. patent application Ser. No. 11/922,279, filed Jun. 1, 2009, which is the U.S. National Stage of PCT/US2006/023034, filed Dec. 21, 2006 which claims priority to U.S. Provisional Patent Application Ser. No. 60/690,017, filed Jun. 13, 2005; U.S. Provisional Patent Application Ser. Nos. 60/690,033, filed Jun. 13, 2005 and Ser. No. 60/796,434, filed May 1, 2006; the entire disclosures of which are incorporated herein by reference in their entirety.

### STATEMENT OF FEDERALLY SPONSORED RESEARCH

Funding for the present invention was provided in part by the Government of the United States by virtue of Grant No. RO1 NS045140-03 by the National Institutes of Health. Thus, the Government of the United States has certain rights in and to the invention claimed herein.

### FIELD OF THE INVENTION

The present invention relates to novel approaches for grafting of human neural stem cells into the adult spinal cord with increased cell survival, differentiation and structural integration of neural stem cells.

### BACKGROUND OF THE INVENTION

Degenerative and traumatic diseases of the nervous system are featured by loss of neurons and their connections. Effective treatments for these conditions are presently unavailable. In the field of experimental therapeutics two major approaches have been taken, i.e. prevention of cell death by interfering with decision-making steps in cell death pathways and the replacement of dead neurons with neural grafts [1,2]. Neural stem cells (NSCs) are a promising alternative to fetal tissues for cell replacement therapies, primarily due to their self-renewal and pluripotentiality [3,4]. The use of human-derived NSCs has the additional advantage of yielding translational information that is relevant to clinical therapeutics.

The adult spinal cord represents an especially challenging environment for the survival and differentiation of NSCs because of the apparent lack of cells and/or signals promoting regeneration [5]. For the most part, NSC grafts into the adult injured cord have either shown poor differentiation [6,7] or a restricted differentiation into the glial lineage [8,9], the latter attributable to the relative preference of pluripotent precursors for non-neuronal fates. However two studies, the one using ES/embryonic body-derived NSCs [10] and the other lineage-restricted neuronal progenitors [11] have achieved good neuronal differentiation of grafted cells, thus mounting a challenge to the notion of spinal cord as an environment unfavorable to neuronal differentiation.

The potential of NSCs as therapies for motor neuron disease will depend on their ability to survive, differentiate, and become integrated when grafted into spinal cords undergoing chronic degenerative changes. However, neurodegeneration represents a particularly challenging biological environment and cell death signals present in established neurodegenerative disease [12-14] may be incompatible with graft survival. In addition, the adult spinal cord is viewed as lacking cells and/or signals required for regeneration [15] and the majority of NSC grafting studies have shown poor or restricted differentiation [16,17]. As mentioned above, recent findings using human NSCs have rekindled some optimism, but these data are limited to intact animals or animals with spinal cord injuries.

One particular motor neuron disease is Amyotrophic lateral sclerosis (ALS) which is characterized by progressive loss of motor neurons in the spinal cord and brain stem, as well as some pyramidal neurons in motor cortex, leading to muscle atrophy and eventual paralysis and death. Approximately 10-13% of cases of ALS are familial and 20% of these cases harbor mutations in the Cu/Zn superoxide dismutase (SOD1) gene [18,19]. Transgenic mice expressing mutant SOD1 with a substitution of alanine for glycine at position 93 (SOD1-G93A) reproduce symptoms and pathologies of human ALS [20-22]. Despite progress in the elucidation of several cellular and molecular mechanisms operating in familial ALS, there is presently no disease-modifying strategy that would prevent the progressive motor neuron death in familial or sporadic forms of the illness [18]. Therefore, neuronal replacement therapies with cell grafts are as applicable in ALS as they are in other diseases featured by neuronal cell death [23-25]. Our neuronal replacement capabilities have been recently expanded with the availability of neural stem cells (NSCs) derived ex vivo from neural tissues or in vitro from embryonic stem cells (ES) as alternatives to fetal grafts [24,25].

Human NSCs, i.e. cells that may eventually be used in clinical applications, are different from their rodent counterparts in important ways. For example, human NSCs proliferate and differentiate slower than rodent cells, and this property may alter the extent and/or rate of migration, axonal elongation, and synapse formation [26-28]. Therefore, prior to their consideration as potential ALS therapy, human NSCs must be studied in vivo in the best available models of motor neuron disease, i.e. transgenic (Tg) rodents harboring SOD1 mutations. A significant obstacle here is the rejection, by rodent hosts, of xenografts from species as distant as humans [29]. The use of cyclosporin A as a single immunosuppressive agent has failed or partially failed in other laboratories [30, 31], and our own [32, 33].

It would be highly desirable to develop methods of promoting large scale structural integration, differentiation and survival of neural stem cells into diseased and injured spinal cords for purposes of restoring normal function.

Throughout this application, various publications are referenced to by numbers. Full citations for these publications may be found at the end of the specification immediately following the Abstract. The disclosures of these publications in their entireties are hereby incorporated by reference into this application in order to more fully describe the state of the art to those skilled therein as of the date of the invention described and claimed herein.

### SUMMARY OF THE INVENTION

The present invention provides methods for treating spinal cord diseases and injuries. The methods involve tranplanting neural stem cells which have been previously expanded in vitro into a patient such that the cells can ameliorate the disease or injury. The stem cells to be transplanted are derived from spinal cord tissue. In one respect, the present invention discloses various stem cells, neural progenitors and neural precursors beneficial for treating various spinal cord diseases or injuries. In another respect, the present invention discloses how to identify, isolate, expand, and prepare the neural stem cells for treatment of spinal cord diseases, disorders or injuries. In particular, the present invention discloses the method of treating degenerate diseases, such as ALS, by transplanting in vitro expanded multipotential neural progenitors or neural stem cells isolated from spinal cord back into a spinal cord.

The cells of the present invention include cells that, upon transplantation, generate an amount of neurons sufficient to operate within the neuronal infrastructure to ameliorate a diseased or injured state. For example, transplantation can be used to improve ambulatory function in a patient with traumatic spinal cord injury. The cells of the present invention undergo neuronal differentiation in the presence of factors existing in an injured tissue. In certain instances, the neural precursor cells can be multipotential neural stem cells capable of expansion in culture and of generating both neurons and glia upon differentiation.

Using the present methods, neural circuits can be treated by transplanting the cells into appropriate regions for amelioration of the disease or condition. Generally, transplantation occurs into spinal cord tissue. In some instances, transplantation can occur into remote areas of the body and the cells can migrate to their intended target. The method of treatment includes supplying to an injured spinal cord area, via transplantation, a suitable number of cells of this invention which can differentiate into a sufficient number of GABA-producing neurons to attenuate defective neural circuits, including hyperactive neural circuits. The cells can be either undifferentiated, pre-differentiated or fully differentiated in vitro at the time of transplantation. The cells can be obtained from fetal, neonatal, juvenile, adult, or post-mortem tissues of the human spinal cord.

Additional features and advantages of the present invention are described in, and will be apparent from, the following Detailed Description of the Invention and the figures.

### BRIEF DESCRIPTION OF THE FIGURES

FIGS. 1A-1D illustrate In vitro differentiation of human NSCs used for grafts;

FIG. 1A. Shows that the vast majority of cells express the NSC-specific marker nestin (red) immediately before grafting. The DNA dye DAPI (blue) was used to reveal all cells in culture; FIGS. 1B-1C Show that fourteen days within the differentiation phase (i.e. after bFGF removal), ˜50% of cells acquire MAP2 immunoreactivity and neuronal cytology with characteristic processes (red, FIG. 1B). A smaller number of cells differentiate into GFAP (+) astrocytes (green, FIG. 1C); FIG. 1D Depicts real-time RT-PCR data showing increased neurotrophic factor and neuregulin expression in the course of NSC differentiation in vitro. Number of days on top of columns is the days NSCs have been in a phase of differentiation (after withdrawal of FGF). Results are expressed as fold increases compared to levels expressed at the proliferation phase (Day 0), the latter values designated as 1. Data represent average±SD of triplicate measurements of a representative cell culture sample at a given time point. Experiment was repeated twice with different sets of cell samples and yielded very similar results. Scale bars: 50 μm.

FIGS. 2A-2F illustrate the survival and migration of human NSCs in rat spinal cord; FIGS. 2A-2C illustrate the localization and numbers of HNu (+) cells at different time points post-grafting, whereas FIGS. 2D-2E support the migratory phenotype of grafted HNu (+) cells and FIG. 2F confirms their low mitotic activity; FIGS. 2A-2B Show that at 3 weeks postgrafting most HNu (+) cells, indicated as red profiles with an arrow in FIG. 2A, are located around the injection sites and along needle tracks. By 6 months (FIG. 2B), HNu (+) cells show widespread migration away from the injection site in both the gray and white matter, and many are seen in the white matter and a few in the gray matter of the contralateral side. FIG. 2B is a composite of several fields to show the extent of migration. Arrow in FIG. 2B shows the colonization, by NSC-derived cells, of the CNS portion of the dorsal root (note the CNS-PNS transition zone); FIG. 2C depicts bar diagrams showing HNu (+) cell numbers at the time of grafting (0) and at 3 weeks (3 w), 3 months (3 m), and 6 months (6 m) post-grafting in the different treatment groups. Left-hand diagram shows numbers of HNu (+) cells ipsilateral to the grafting site (Ipsi), and right-hand diagram shows numbers on the contralateral grey matter (Contra). Brackets show the results of post-hoc testing when ANOVA was significant in the avulsion and HCA groups ipsilateral to grafting; in all other cases, significance was established with a students' t test. Asterisk indicates statistical significance at p<0.05 by ANOVA or t test. Method of section selection is indicated on the extreme left; FIG. 2D Shows that Dcx, a marker for migrating neuronal precursors, was expressed by about 80% of grafted cells three weeks post-grafting. Dcx expression is reduced to 10-15% of HNu (+) cells surrounding the grafting sites at 3 and 6 months but remains very high (˜80%) in HNu (+) cells on the contralateral gray matter up to 6 months post-grafting; FIG. 2E is a confocal image of HNu (+) (red) cells also labeled with Dcx (green) at 3 weeks post-grafting; FIG. 2F depicts three panels illustrating, on a section that was dually stained for HNu (red nuclear marker in left panel) and Ki67 (green nuclear marker in central panel), the very low rate of mitotic activity (double-stained nuclei in right panel) in NSC grafts. The single double-stained nuclear profile is indicated with an arrow. Scale bars: A, 200 μm; B, 600 μm; E, 10 μm; F, 20 μm.

FIGS. 3A-3E illustrate the differentiation of grafted human NSCs into neurons and astrocytes; FIGS. 3A-3D illustrate cases of neuronal (FIGS. 3A-3B) and astrocytic (FIGS. 3C-3D) differentiation of HNu (+) cells by epifluorescence (FIG. 3A and FIG. 3C) or confocal (FIG. 3B and FIG. 3D) microscopy. FIG. 3E combines bar diagrams to illustrate the fate of NSC grafts at parenchymal (left-hand) and meningeal (right-hand) sites; FIGS. 3A-3B are two sections, dually stained for HNu and TUJ-1, which illustrate the very high frequency of NSC-derived neurons within the parenchyma of the ventral horn by epifluorescence (FIG. 3A) and confocal microscopy (FIG. 3B). Both preparations are taken from animals killed 3 months post-grafting. Inset is a magnification of demarcated area in (FIG. 3A). Note the homogeneous appearance of TUJ-1 (+) cells in the FIG. 3A inset. Confocal sections have been virtually re-sectioned at the x and y planes to confirm the identity of the double-stained structure; FIGS. 3C-3D are sections, dually stained for HNu and GFAP, which illustrate the significant astrocytic differentiation of NSCs located on to pia-arachnoid by epifluorescence (FIG. 3C) and confocal microscopy (FIG. 3D). Inset is a magnification of demarcated area in (FIG. 3C) and representative astrocytes are indicated by arrows. Confocal sections have been processed as in (FIG. 3B); FIG. 3E depicts bar diagrams illustrating the phenotypic fates of human NSC grafts into the parenchyma (left-hand diagrams) or the meninges (right-hand diagrams) at 3 weeks (3 w), 3 months (3 m), and 6 months (6 m) in different treatment groups. Neuronal fate is represented by numbers of TUJ-1-labeled HNu (+) cells, and astrocytic fate is represented by numbers of GFAP-labeled HNu (+) cells. NSCs in a neural stem/precursor state are depicted here as nestin-and-HNu double-labeled cells. Asterisk indicates critical post-hoc differences in groups with significant variance (p<0.05). Although some experimental protocols appear to bias NSC differentiation (see the robust neuronal fate of NSCs in the avulsion group), these trends are not significant P: parenchymal location; M: meningeal location; Scale bars: FIGS. 3A and 3C, 20 μm; FIGS. 3B and 3D, 10 μm.

FIGS. 4A-4B illustrate the differentiation of human NSCs into neurons after grafting into the lumbar spinal cord of normal adult Sprague-Dawley rats; Panels illustrate the neuronal differentiation of NSCs two months post-grafting based on dual-label immunofluorescence for HNu (red) and a neuronal marker (green, FIG. 4A: TUJ1; FIG. 4B: NeuN). The predominance of double-labeled profiles in both panels matches the avid neuronal differentiation of human NSCs in nude rats as illustrated in FIGS. 3A-3E; Scale bars: 20 μm.

FIGS. 5A-5E and FIGS. 5A′-5E′ illustrate neurotransmitter differentiation of grafted human NSCs. FIGS. 5A-5E and FIGS. 5A′-5E′ illustrate evidence of glutamatergic (FIG. 5A and FIG. 5A′), GABAergic (FIG. 5B, FIG. 5B′, FIG. 5C and FIG. 5C′), and cholinergic (FIG. 5E and FIG. 5E′) neurotransmission in NSC grafts. As in previous figures, confocal microscopy is used primarily to confirm the colocalization of two markers in the same cellular compartment along three planes of sectioning; FIG. 5A and FIG. 5A′ illustrate two sections, stained for HNu and the prevalent AMPA receptor epitope GluR2/3, show both cytoplasmic and synaptic staining by epifluorescence (FIG. 5A) or confocal (FIG. 5A′) microscopy. Insets in (FIG. 5A) represent magnifications of indicated neurons in main panel; top and bottom-left insets show two medium-size HNu (+) cells with cytoplasmic immunoreactivity, whereas bottom-right inset illustrates a larger HNu (+) cell containing multiple GluR2/3 (+) boutons; FIG. 5B and FIG. 5B′ depict sections stained for HNu and the GABA-synthesizing enzyme GAD and visualized with epifluorescence (FIG. 5B) or confocal microscopy (FIG. 5B′). Arrows in (FIG. 5B) indicate multiple HNu (+) cells with cytoplasmic GAD immunoreactivity; FIG. 5C and FIG. 5C′ show confocal microscopy of a field stained with both human synaptophysin (red in single-channel image on top left, to label graft-derived terminals) and GAD (green in single-channel image on bottom left, to label GABAergic terminals) shows colocalization of the two proteins (yellow color in merged images in FIG. 5C′) in multiple synaptic boutons. Nearly all graft-derived boutons are inhibitory (FIG. 5C′); FIG. 5D and FIG. 5D′ sections (FIG. 5D, epifluorescence; FIG. 5D′, confocal) stained for human synaptophysin to label graft-derived terminals (red) and mixed VGLUT-1/VGLUT-2 antibodies to label glutamatergic terminals in the field (green). Despite significant overlap and apposition of graft-derived and VGLUT-1/2 (+) terminals (FIG. 5D), the two groups of terminals do not overlap (FIG. 5D′); FIG. 5E and FIG. 5E′ illustrate two sections that were dually stained for HNu and ChAT (FIG. 5E and the insert, epifluorescence; FIG. 5E′, confocal microscopy), some of the largest NSC-derived neurons express cholinergic phenotypes. These cells elaborate multiple primary dendrites (FIG. 5E and the insert). FIG. 5E′ is the confocal image; Scale bars: FIGS. 5A, 5B, 5D, 5E, 20 μm; FIGS. 5A′, 5B′, 5C, 5C′, 5D′, 5E′, 10 μm.

FIGS. 6A-6H illustrate the differentiation of grafted human NSCs into phenotypes associated with developing motor neurons; FIG. 6A and FIG. 6E illustrate the expression, in El 3 rat motor neurons, of islet-1 and p75^{NTR}, respectively. FIG. 6B illustrates the expression of islet-1 in host motor neurons (adult). FIGS. 6A-6B and FIG. 6E serve as positive controls for staining seen in graft-derived cells in FIGS. 6C-6D and FIGS. 6F-6H; FIGS. 6A-6D show that many developing (FIG. 6A) and some adult (FIG. 6B) motor neurons express nuclear islet-1 immunoreactivity (red). A portion of HNu (+) cells in spinal cord are also islet-1 (+) (FIG. 6C; arrows), but staining is predominantly cytoplasmic. This staining is not present when primary antibody is replaced with pre-immune IgG and may signify either leakage or a different processing of this protein by NSC-derived cells. Colocalization of islet-1 with the graft-specific marker HNu in same neurons is confirmed with confocal microscopy (FIG. 6D); FIGS. 6E-6H show that our p75^{NTR} ICC protocol stains the cytoplasm of developing motor neurons (green; FIG. 6E) as well as a large number of graft-derived, HNu (+) neurons in the adult spinal cord (FIG. 6F). FIGS. 6F-6H represent sections multiply labeled for HNu (blue), p75^{NTR} (red) and ChAT (green). Besides a proportion of p75^{NTR}(+) HNu (+) neurons express p75^{NTR} immunoreactivity (FIG. 6F). FIG. 6H represents a confocal microscopic image that was virtually re-sectioned in the x and y planes to show the colocalization of p75^{NTR} and ChAT in selective graft-derived, HNu (+) neurons; Scale bars: FIGS. 6A, 6E, 100 μm; FIGS. 6B, 6C, 6F, 6G, 20 μm; FIGS. 6D, 6H, 10 μm.

FIGS. 7A-7D illustrate the maturation of human NSC-derived neurons based on the elaboration of axons, synapses, and innervation by host neurons; FIG. 7A is a field taken through the ventral horn of a HNu/NF-70 stained section 3 months post-grafting and shows bundles of human NF70 (+) axons (indicated with white arrows) originating in HNu (+) grafts (one indicated with an asterisk on top right) and converging to form larger pathways (red arrows on bottom left) that course towards the ventral white matter; FIG. 7B is a field taken from a graft site in the ventral horn of a human synaptophysin-stained section 3 months post-grafting. The sharp boundaries of staining of puncta only in the graft region (boundaries demarcated with arrows) are due to the selectivity of the antibody for human, but not rat, synaptophysin protein; FIGS. 7C-7C′ (FIG. 7C, epifluorescence; FIG. 7C′, confocal) are taken from triple-stained sections with HNu (red), TUJ-1 (blue), and the presynaptic marker Bassoon (Bsn, green). The Bassoon antibody used for ICC recognizes rat and mouse, but not human, protein. FIG. 7C depicts a dense field of rat Bassoon (+) terminals in proximity to HNu and TUJ-1 (+) profiles. Specific contacts between rat terminals and NSC-derived neurons are indicated with arrowheads in the inset, which is a magnification of the profile at the center of the main panel. The very large number of such terminals on NSC-derived cell bodies is best illustrated with confocal microscopy (FIG. 7C′); FIGS. 7D-7D′ (FIG. 7D, epifluorescence; FIG. 7D′, confocal) illustrate sections stained with HNu (red), TUJ-1 (blue), and mixed VGLUT1/VGLUT2 antibodies (green) and show the innervation of HNu and TUJ-1 (+) cells by glutamatergic terminals putatively originating in the host; Scale bars: FIG. 7A, 80 μm; B, 20 μm; FIGS. 7C-7D, FIGS. 7C′-7D′, 10 μm.

FIGS. 8A-8B illustrate the innervation of host neurons (including motor neurons) by graft-derived nerve cells; FIGS. 8A-8B (FIG. 8A and inset, epifluorescence; FIG. 8B, confocal) illustrate sections stained with human synaptophysin (red) and TUJ-1 (green) and show the innervation of host motor neurons (large TUJ-1 [+] cell bodies) by human synaptophysin (+) terminals originated from grafted NSCs. FIG. 8A shows the site of the original graft (area with dense human synaptophysin staining pointed with an arrow) away from the area of innervation (enlarged in inset); Scale bars: FIG. 8A, 200 μm; FIG. 8B, 20 μm.

FIGS. 9A-9C illustrate root trajectories of NSC-derived axons in animals with intact peripheral conduits. Cases illustrated here are taken from animals with HCA lesions in L5 motor neurons (FIG. 9A) and show L5 roots containing both axons and migrating NSC-derived cells (FIGS. 9B-9C) marked with human-specific antibodies; FIG. 9A shows two representative cresyl violet preparations of L5 ventral horn from an intact (top) and an HCA lesioned animal (bottom). Note the degeneration of α-motor neurons 1 week after the local application of the excitotoxic compound (arrows); FIG. 9B shows a teased L5 root whole mount preparation stained with both HNu (nuclear staining) and human NF70 (axonal staining) antibodies shows prolific elaboration of NSC-derived axons in some distance into the root. The spinal (cord) and nerve (periphery) sides of the root are denoted. A significant number of HNu (+) migrate along the axonal tracts, especially proximal to the cord (bottom right); FIG. 9C is a cross-sectional preparation of L5 ventral root stained with HNu (for nuclei of grafted NSCs) and human NF70 (for NSC-derived axons). Both are visualized with DAB (brown). Note both NSCs (asterisks) and axons (an example is given with an arrow) in the endoneurium, wedged among host motor axons (unstained round structures). Insets represent magnifications of indicated fields; Scale bars: FIG. 9A, 80 μm; FIGS. 9B-9C, 100 μm.

FIGS. 10A-10D illustrate the Survival and neuronal differentiation of human NSCs in the spinal cord of SOD1 G93A rats; FIG. 10A is a section taken from a representative graft site of an end-stage animal and stained with HNu ICC (red). A large number of HNu (+) cells are shown to survive at the original graft site in ventral horn/ventral root exit zone (arrows); FIGS. 10B-10D are fields photographed from a section dually stained with HNu (red) and the neuronal marker TUJ1 (green) and show that a majority of HNu (+) cells in this ventral L5 graft are also TUJ1 (+). FIG. 10C represents an enlargement of the framed area in FIG. 10B and FIG. 10D is a confocal image taken from the framed area in FIG. 10C; Image in FIG. 10D was optically resectioned perpendicular to the z plane to confirm the colocalization of TUJ1 (+) cytoplasm and HNu (+) nucleus at the same plane. Blue emission channel for DAPI counterstain is merged only in FIG. 10D; Size bars: FIG. 10A, 150 μm; FIG. 10B, 100 μm; FIG. 10C, 20 μm; FIG. 10D, 10 μm.

FIGS. 11A-11G illustrate the structural integration of human NSCs in the spinal cord of SOD1 G93A rats; Images depict patterns of graft-to-host innervation (FIGS. 11A-11B), host-to-graft innervation (FIGS. 11C-11D), and pathways of graft-derived axons forming within the host spinal cord (FIGS. 11E-11H); FIGS. 11A-11B are images taken from a section that was dually stained for ChAT (green) and human-specific synaptophysin (red) ICC at lower (FIG. 11A) and higher (FIG. 11B, confocal) magnifications. Confocal image in FIG. 11B was taken from the framed area in FIG. 11A. Section in FIG. 11B was counterstained with DAPI (blue). As shown for many host motor neurons in FIG. 11A and with a confocal level of detail in FIG. 11B, cell bodies and proximal dendrites of host α-motor neurons are contacted by a large number of graft-derived boutons. In FIG. 11B, several of these synaptic contacts are confirmed with x- and y-plane reconstruction (arrows); FIGS. 11C-11D are photographs taken from two sections processed with triple immunofluorescence for HNu (blue), TUJ1 (green), and the rat form of the presynaptic protein Bassoon (red). Section in FIG. 11C was photographed under epifluorescence. FIG. 11D represents a confocal detail from an adjacent section. Note a general overlap of host-derived terminals (red) with a TUJ1 differentiated graft in FIG. 11C (arrows). When examined in greater detail, i.e. with confocal microscopy (FIG. 11D), only few graft-derived neurons are seen to receive synaptic contacts by host cells (asterisk in main frame, arrow in x-plane reconstruction). FIGS. 11E-11E′ are digital photographs, taken from a horizontal section stained for HNu and human NF-70 ICC (both red), counterstained with ChAT ICC (green) and processed for the acquisition of red emission (FIG. 11E) or merged red and green emission (FIG. 11E′). A large number of graft-derived axons are shown in panel E to leave the graft on top and course preferentially along the left border of this field (arrows). The superimposition of green ChAT immunostaining in cells/processes in FIG. 11E′ serves to indicate the position of host motor neurons in the ventral horn (asterisks) and to define the position of graft-derived pathway in the white matter of the ventral funiculus; FIG. 11F. In all cases with live grafts, there was a prominent visualization of human axons and synapses around ependymal cells. Although a de novo projection of NSC-derived neurons to ependyma could not be ruled out, the majority of these processes resulted from migration of NSCs to ependymal sites. FIG. 11F illustrates human synaptophysin (+) synapses apposed to DAPI-stained ependymal cells; FIG. 11G is a teased preparation from ventral L5 root which was stained en block with human NF-70 and shows sparse graft-derived axons coursing near the surface (arrows); Size bars: FIG. 11A, 50 μm; FIG. 11B, 10 μm; FIG. 11C, 10 μm; FIG. 11D, 20 μm; FIG. 11E, 100 μm; FIG. 11F, 10 μm.

FIGS. 12A-12E illustrate the Effects of human NSC treatment on severity of motor neuron disease in G93A SOD1 rats shown with progression (FIGS. 12A-12B) as well as end-point (FIGS. 12C-12E) analysis of clinical and pathological measures in cases with live-cell (L) and dead-cell (control, C) grafts; FIG. 12A is a Kaplan-Meier plot showing a significant separation between experimental (n=16) and control animals (n=11) throughout the course of observation (p=0.0003). FIG. 12B shows a separation in the two principal measures of muscle weakness (BBB and inclined plane scores) between the two groups (p=0.00168 and 0.00125, respectively); FIGS. 12C-12E are end-point analysis of survival (FIG. 12C), time-to-disease-onset (FIG. 12D), and motor neuron numbers based on Niss1 staining (FIG. 12E), in experimental (n=3) and control rats (n=3). Bar diagram in FIG. 11C shows a significant 11-day difference in life span between the two groups (p=0.0005), and diagram in FIG. 12D indicates a significant 7-day difference in time-to-disease-onset between the two groups (p=0.0001). FIG. 12E depicts a difference of 3,212 cells in the lumbar protuberance between live and dead NSC groups (p=0.01). Inset at the bottom of FIG. 12E illustrates the difference in motor neuron survival between a representative experimental (upper) and control (lower) rat at 128 days of age; arrows indicate the lateral motor neuron group; Size bars: 150 μm.

FIGS. 13A-13D illustrate the expression and release of GDNF and BDNF in the spinal cord of NSC-grafted SOD1 G93A rats; FIG. 13A shows GDNF concentrations in the parenchyma and CSF of rats grafted with live cells (L1 and L2 bars n=3) and animals grafted with dead cells (C bar, n=3) by ELISA; L1 bars represent concentrations through the graft site; L2 bars depict concentrations in tissues one segment above or below. Average GDNF concentration was 0.912 pg/μg at the graft site and 0.819 pg/μg one spinal segment away in animals grafted with live cells; in animals that received dead cells, average graft-site concentration was 0.368 pg/μg (left); In the CSF, GDNF concentration was 0.027 pg/μl in experimental and 0.006 pg/μl in control animals (right). Variance in parenchymal concentrations among groups is significant and caused by a large difference between L1 or L2 and C groups. Difference in CSF concentrations between experimental (live-cell, L) and control (dead-cell, C) groups are also significant by t test; FIG. 13B is a GDNF Western blotting, serving as confirmation of ELISA, which detects a 16 kDa protein (left) and shows a higher normalized GDNF concentration in animals grafted with live NSCs; FIG. 13C depicts BDNF concentrations in the parenchyma and CSF of experimental rats (L1 and L2 bars, n=3) and controls (C bars, n=3) by ELISA. BDNF concentration was 0.086 pg/μg at the graft site and 0.054 pg/μg one segment away in animals grafted with live cells. In rats grafted with dead cells, graft-site concentration was 0.010 pg/μg. In the CSF, BDNF concentration was 0.041 pg/μl in animals with live cells and 0.010 pg/μl in animals with dead cells. Variance in these values is significant because of large differences between live- and dead-cell grafts, but also between graft sites and sites adjacent to them in animals with live NSCs (left). Differences between experimental and control CSF concentrations are also significant (right); FIG. 13D depicts BDNF and GDNF real time RT-PCR and demonstrates that spinal cord tissues with human NSC grafts express higher levels of human, but lower or unchanged levels of rat BDNF and GDNF mRNA. Melting curve and agarose gel analysis detected single melt peaks and specific bands for each of the eight rat and human-specific primers (data not shown). PCR efficiency values were between 92 and 105% for all amplicons. RT minus, NTC, rat cDNA (for human-specific primers), and human NSC cDNA (for rat-specific primers) control reactions did not amplify any specific product. The normalized expression ratio of human BDNF and GDNF in grafts is ˜8-fold and 9-fold higher, respectively, compared to levels of expression in NSCs prior to grafting. In contrast, host tissues with live NSC grafts express ˜3.5 times lower BDNF, whereas GDNF expression does not change compared to control tissues grafted with dead NSCs; * p<0.05 ** p<0.01

FIGS. 14A-14F illustrate the localization of GDNF immunoreactivity in cell bodies (FIGS. 14A-14D) and terminals (FIG. 14E) of NSC-derived neurons in the spinal cord of SOD1 G93A rats; FIGS. 14A-14B. are dually stained preparations for HNu (red) and GDNF (green) which illustrate the abundance of GDNF immunoreactivity within the cytoplasm of grafted NSCs under low-power epifluorescence. FIG. 14A was photographed in multiple emission acquisition channels such as to allow for the visualization of green only (left), red only (middle), and merged red and green (right) epifluorescence. FIG. 14B was taken from a section adjacent to the one in FIG. 14A that was stained for HNu and rabbit IgG immunoreactivity to control for GDNF antibody background; image in FIG. 14B is the product of merged green and red channels. Note the delineation of the graft area by intense GDNF immunoreactivity in FIG. 14A and the abolition of specific immunoreactivity when anti-GDNF is replaced with pre-immune rabbit IgG (FIG. 14B). FIG. 14C-14D are two merged-emission photographs that serve to illustrate in greater detail the cytoplasmic GDNF immunoreactivity of NSC grafts. Red and green emissions represent HNu and GDNF immunoreactivity, as in FIGS. 14A-14B. Confocal image in FIG. 14D is taken from the framed area of the epifluorescent image in FIG. 14C. Confocal image was optically resectioned at the x and y planes to confirm GDNF and HNu colocalization; FIGS. 14E-14F are images taken through the central canal region of sections dually stained for human synaptophysin (red) and GDNF (green) and photographed under epifluorescent (FIG. 14E) or confocal (FIG. 14F) conditions for the acquisition of merged emission of red and green fluorescence and blue emission from DAPI staining. Note the precise colocalization of human synaptophysin, serving as a graft-specific synaptic marker, and GDNF immunoreactivity in the dense terminal field near ependymal cells (asterisks). Colocalization is especially evident with confocal microscopy in FIG. 14F (index double-labeled synapses are indicated with arrows); Size bars: FIG. 14A, FIG. 14B 150 μm; FIG. 14C, 50 μm; FIG. 14D, 10 μm; FIG. 14E, 10 μm.

FIGS. 15A-15E illustrate the localization of GDNF immunoreactivity in synapses associated with host motor neurons in SOD1 G93A rats at different stages of disease progression. FIG. 15A is a representative illustration of GDNF immunoreactivity in motor neurons from animals grafted with live (left and right) and dead (center) NSCs at early and late-stage disease. Note the high density of GDNF (+) boutons, especially on the cell bodies and proximal dendrites of host motor neurons in early disease (left, arrows). These terminals are smaller with advanced disease (right, arrows). There is also granular GDNF immunoreactivity within the cytoplasm, best appreciated in confocal images (FIG. 15C′); FIGS. 15B-15C′ are confocal images of a host motor neuron stained with GDNF (green) and human synaptophysin (red; to selectively label NSC-derived terminals). FIG. 15B, corresponding to early motor neuron disease, shows the rare localization of GDNF within human synaptophysin (+) synapses (arrow). In late disease (FIGS. 15C-15C′), most immunoreactive GDNF appears in vesicular structures within host motor neurons (arrows in FIG. 15C′). FIG. 15C′ is an enlargement of the framed area in FIG. 15C; FIGS. 15D-15E are confocal images of a representative host motor neuron from sections dually stained with GDNF and ChAT or GDNF and VAChT. ChAT and VAChT are cholinergic markers, the former labeling both cholinergic cell bodies and terminals and the latter labeling local cholinergic terminals. GDNF immunoreactivity is detected with green and ChAT and VAChT with red emission. The cholinergic cell body is contacted by multiple ChAT or VAChT and GDNF (+) large terminals with the appearance of cholinergic C-boutons (arrows). X and y plane reconstruction verifies the colocalization of ChAT or VAChT and GDNF on these terminals (arrows); Size bars: FIG. 15A, 20 μm; FIG. 15B, 10 μm; FIG. 15C, 10 μm; FIG. 15D, 10 μm; FIG. 15E, 10 μm.

FIGS. 16A-16F illustrate the effects of various immunosuppressive regimens on graft survival versus CD8 cell infiltration. Spinal cord sections were taken through the graft site and dually labeled with antibodies against HNu (red) and CD8 (green). Images were captured under epifluorescence, except in top inset of FIG. 16A which is a confocal image taken from the same section as the main panel. All insets, except the top one in FIG. 16A, represent magnifications of the framed areas in main panels. Arrows in insets point to representative CD8 (+) T-cells; Arrowheads point to CD8 (+) cellular debris. Cross-sections of some blood vessels are labeled with asterisks. FIGS. 16A-16B illustrate representative sections of mice treated with FK506, surviving for 1 week (FIG. 16A) or 1 month (FIG. 16B) post-grafting. Note many intact CD8 (+) cells in (FIG. 16A) with their typical thin cytoplasm. Many CD8 (+) cells are in close proximity to diffuse HNu immunoreactivity that gives the impression of scaffolding around green CD8 (+) profiles (arrows in insets in FIG. 16B); At 1 month, CD8 immunoreactivity is localized as predominantly extracellular debris (arrowheads in inset in FIG. 16B); FIGS. 16C-16D show a significant increase in graft survival (indicated by the presence of a dense population of intact HNu [+] nuclei) and a decrease in CD8 (+) cell infiltration with combined FK506+rapamycin treatment at 1 week (FIG. 16C) and 1 month (FIG. 16D) post-grafting. CD8 immunoreactivity is present both in intact cells (arrows in insets) and as extracellular debris (arrowheads in insets). CD8 (+) debris is much less intense than in FIG. 16B, presumably due to a lower frequency of CD8 (+) T-cells recruited into the graft site; FIGS. 16E-16F show robust graft survival and minimal CD8 (+) cell infiltration one month after treatment with combined FK506+rapamycin+MMF (E) or CD4 antibodies (FIG. 16F). The results resemble that of FK506+rapamycin treatment at 1 month (FIG. 16D); Scale bars: main FIGS., 100 μm; all insets (except confocal in FIG. 16A), 20 μm; confocal in FIG. 16A (upper inset), 10 μm.

FIGS. 17A-17B illustrate the effects of FK506 (FIG. 17A) and combined FK506+rapamycin treatment (FIG. 17B) on graft survival versus CD4 cell infiltration one week post-grafting; Spinal cord sections were taken through the graft site and dually stained with antibodies against HNu (red) and CD4 (green). All images were captured under epifluorescence, and insets represent magnifications of framed areas in main panels, except the confocal image in top inset in FIG. 17A. Note the sharp difference between non-nuclear HNu immunoreactivity in FIG. 17A and intense HNu immunoreactivity of densely packed nuclei in FIG. 17B; In FIG. 17A, arrows in insets point to representative CD4 (+) cells. In contrast to CD8 (+) cells in FIG. 1A, most of these cells are not in close proximity to HNu-immunoreactive material. Cross-section of a blood vessel is labeled with an asterisk; In FIG. 17B, arrowheads in inset point to CD 4 (+) cellular debris, which is the predominant CD4-immunoreactive structure in these preparations. Scale bars: main panels, 100 μm; all insets (except confocal in FIG. 17A), 20 μm; confocal in FIG. 17A, 10 μm.

FIGS. 18A-18F illustrate the Effects of various immunosuppressive regimens on graft survival versus microglia/macrophage infiltration; Spinal cord sections were dually stained with antibodies against HNu (red) and Iba-1 (green). Images were captured under epifluorescence, except in top inset of FIG. 18A which is a confocal image. All insets except the top one in FIG. 18A represent magnifications of framed areas in main panels. Arrows in insets point to representative microglial cells/macrophages; FIGS. 18A-18B are an illustration of representative sections of mice treated with FK506 and surviving for 1 week (FIG. 18A) or 1 month (FIG. 18B) post-grafting. Most Iba-1 (+) cells in FIG. 18A show pyramidal shapes with substantial cytoplasm and short processes, i.e. cytological features of macrophages (arrows in insets). Colocalization of HNu (+) material internal to the Iba-1 (+) cell surface is evident in the confocal inset (arrow, top inset in FIG. 18A). Iba-1 (+) cells in panel B show mixed cytologies with both extensively ramified cells resembling activated microglia and some macrophage-like profiles; FIGS. 18C-18D show a significant increase in graft survival (indicated by the presence of a dense population of intact HNu [+] nuclei) with combined FK506+rapamycin treatment at 1 week (FIG. 18C) or 1 month (FIG. 18D) post-grafting. IBA-1 (+) cells are far fewer than in FIG. 18A and are comprised primarily of activated microglia in FIG. 18C, whereas in FIG. 18D they also include macrophage-like profiles; FIGS. 18E-18F show robust graft survival one month after treatment with combined FK506+rapamycin+MMF (FIG. 18E) or CD4 antibodies (FIG. 18F). There are several macrophage-like Iba-1 (+) cells in FIG. 18E. In FIG. 18F, most Iba-1 (+) cells have cytologies consistent with activated microglia; Scale bars: main figures, 100 μm; all insets (except confocal in FIG. 18A), 20 μm; confocal in FIG. 18A, 10 μm.

FIGS. 19A-19D illustrate the combined immunosuppressive drugs or CD4 antibodies delay disease onset, improve motor scores, and extend life span of SOD1-G93A mice grafted with human NSCs and treated with combined immunosuppressants as compared to FK506 monotherapy; FIGS. 19A-19B are graphs indicating that combined immunosuppressive treatments or CD4 antibodies delay disease onset (FIG. 19A) and extend the life span (FIG. 19B) of SOD1 mice. Variance in both measures is significant (ANOVA: p=0.0029 and p=0.068, respectively). Fischer LSD post-hoc testing shows that the significance originates in differences between the combined treatment groups or the CD4 antibody group with the FK506 monotherapy group. Note that the FK506+rapamycin+MMF group is not significantly different from the FK506 group with respect to disease onset; FIGS. 19C-19D depict variance in the progression of muscle weakness (FIG. 19C) and in survival (FIG. 19D) among treatment groups. Muscle strength was scored with open-field testing as explained in Materials and Methods. Repeated-measures ANOVA followed by Fisher LSD post-hoc testing of individual differences in FIG. 19C reveals significant differences comparing FK506+rapamycin or FK506+rapamycin+MMF or anti-CD4 groups to FK506 group (p=0.007, 0.008, and 0.023, respectively). Logrank testing of Kaplan-Meier survival curves in FIG. 19D shows an overall significant variance (p=0.011, χ²=11.11). Using Logrank testing for comparisons between pairs of groups reveals significant differences between anti-CD4 or FK506+rapamycin or FK506+rapamycin+MMF and FK506 monotherapy (p=0.023, 0.016, and 0.001, respectively). Group data are displayed as mean±SD. (** p<0.01, based on post-hoc testing).

FIGS. 20A-20D illustrate the differences in clinical parameters of motor neuron disease between animals grafted with live or dead human NSCs, all of which were optimally immunosuppressed with FK506 plus rapamycin; FIGS. 20A-20B show that live NSC grafts delay disease onset (FIG. 20A) and extend the life span (FIG. 20B) of SOD1 mice when compared to dead-cell grafts. On both measures, differences between the two groups are significant (Student's t-test: p=0.0025 and p=0.012, respectively); FIGS. 20C-20D depict differences in the progression of motor weakness (FIG. 20C) and in survival (FIG. 20D) between live- and dead-cell grafted groups. Repeated-measures ANOVA followed by Fisher LSD post-hoc testing of variance in FIG. 20C shows significant differences between live- and dead-cell grafted groups (p=0.02). Logrank testing of Kaplan-Meier survival curves (FIG. 20D) also shows significant differences between the two groups (p=0.001, χ²=11.11); Group data are displayed as mean±SD. (* p<0.05; ** p<0.01).

FIGS. 21A-21C illustrate the differentiation of human NSCs into neurons and astrocytes in vivo; FIGS. 21A-21A′ are sections which were dually stained for HNu (red) and TUJ1 (green) and illustrate the very high frequency of neurons derived from human NSCs using epifluorescence (FIG. 21A) and confocal microscopy (FIG. 21A′). FIG. 21A is a low-power image illustrating the marked enrichment of TUJ1 immunoreactivity in the graft compared to the surrounding host tissue; Confocal images in FIG. 21A′ illustrate the typical filamentous cytoplasmic TUJ1 immunoreactivity; image in main frame was optically resectioned in the x and y planes to confirm the intimate apposition of the two immunoreactivities within these densely clustered neuronal cell bodies. FIGS. 21B-21B′ are sections stained for HNu (red) and GFAP (green) which showcase the sparse astrocytic differentiation of human NSCs by epifluorescence (FIG. 21B) and confocal microscopy (FIG. 21B′). Note the presence of rare GFAP (+) cell bodies with enclosed HNu (+) nuclei in the graft (arrow in FIG. 21B) despite the presence of numerous GFAP (+) processes. Confocal microscopy (FIG. 21B′) shows a small cluster of human NSC-derived GFAP (+) cells located close to the pial surface. Confocal sections have been processed as in panel A′; C—C′. These sections were stained for HNu (red) and nestin (green) and illustrate that some human NSCs retain stem-cell properties, at least as indicated by their expression of high levels of nestin immunoreactivity by epifluorescence (FIG. 21C) and confocal microscopy (FIG. 21C′); Confocal images have been processed as in panel A′. Arrows in FIG. 21C point to selected nestin (+) cells; Scale bars: FIG. 21A: 50 μm; FIG. 21B, FIG. 21C: 20 μm; FIG. 21A′, FIG. 21B′, FIG. 21C′: 10 μm.

FIGS. 22A-22B illustrate the reciprocal innervation between graft-derived and host neurons based on triple. ICC for human synaptophysin (Syn), two VGLUT epitopes (1 and 2), and TUJ1; Human synaptophysin immunoreactivity is used as a selective marker for graft-derived synapses. VGLUT1/2 is an excitatory synaptic marker specifying host origin because of the absence of glutamatergic phenotypes in differentiated NSCs. TUJ1 is a generic neuronal marker. Color representations of various immunoreactivities are specified on top of panels; FIGS. 22A-22A′ are epifluorescent (FIG. 22A) and confocal (FIG. 22A′) images taken through the ventral horn of a SOD1-G93A mouse one month post-grafting and show a TUJ1 (+) (green) host α-motor neuron (outlined by the three arrows in FIG. 22A) contacted, at both the cell bodies and dendrites, by many human synaptophysin (+) terminals deriving from differentiated NSCs (red). DAPI (blue) is used as nuclear counterstain; FIGS. 22B-22B′ are epifluorescent (FIG. 22B) and confocal (FIG. 22B′) photographs taken through the graft site and show graft-derived neurons, identified by HNu (red) nuclei and cell body staining of TUJ1 (blue) in proximity to VGLUT1/2 (+) (green) terminals from host neurons (some are indicated with arrows in FIG. 22 B). Confocal images have been optically resectioned in the x and y planes as explained in FIG. 5A′; Scale bars: FIG. 22A, FIG. 22B: 20 μm; FIG. 22A′: 10 μm; FIG. 22B′: 5 μm.

FIGS. 23A-23B illustrate the effects of FK506 (FIG. 23A) and FK506+rapamycin (FIG. 23B) treatment on graft survival versus NK-cell infiltration one week post-grafting; Spinal cord sections were dually stained with antibodies against HNu (red) and NK cells (green). Images were captured under epifluorescence and insets represent magnifications of framed areas in main panels, except the top inset of FIG. 23A that is a confocal image from the same section from which the image in the main figure was obtained; In FIG. 23A, arrows in insets point to DX5 (+) NK cells. NK cells come into close contact with non-nuclear HNu immunoreactivity that is often seen in a scaffold pattern around NK cells and likely derives from dead human cells; FIG. 23B depicts, in contrast to FIG. 23A, a large number of densely packed HNu (+) nuclei indicates robust graft survival. Arrowheads in inset point to DX5 (+) NK cell debris, that appears very early in treatments with combined immunosuppressants or CD4 antibodies; Scale bars: main panels, 100 μm; all insets (except confocal image in FIG. 23A), 20 μm; confocal in FIG. 23A, 10 μm.

### DETAILED DESCRIPTION OF THE INVENTION

### Integration and Differentiation of Human Neural Stem Cells in Adult Spinal Cord

The present invention provides for large-scale differentiation and integration of human NSCs grafted into the normal and injured spinal cord of T-cell deficient (nude) rats. Under the present experimental conditions, human NSCs survive well with limited further mitotic activity and migrate extensively for at least 6 months post-grafting. Although the vast majority of parenchymally-grafted NSCs take on a neuronal fate, the meningeal environment appears to promote an astrocytic differentiation or to restrict NSCs to a perpetual nestin (+) state. A majority of NSC-derived neurons in spinal cord parenchyma have bipolar cytologies and GABAergic phenotypes for at least 6 months after grafting and receive GABAergic innervation from other graft and host neurons, in addition to glutamatergic innervations from the host. A small percentage of graft-derived neurons evolve into larger multipolar neurons with cholinergic phenotypes. NSC-derived neurons elaborate axons and synaptic specializations and engage in dense reciprocal innervation with host spinal cord neurons. The present invention relates to parenchymal NSC grafts which integrate into neural circuits in spinal cord and that NSCs of human origin can be useful in methods for repairing damaged spinal circuitry.

The 3-4 fold increase in NSC numbers post-grafting, although not of the magnitude or rate to cause tumors, persisted during the period of active neuronal differentiation. Because it is implausible that the differentiation and dividing pools of NSCs are the same cell population, a subset of grafted NSCs persists in a nestin (+) state, for example NSC-derived cells located near the pial surface, is the one that gives rise to additional neuronal lineage, possibly on an ongoing basis. These cells can be persisting in a premature stage either because of accidental initial placement near the pial environment or via active migration and tropism to those pial sites. These are complex phenomena that require coordinated NSC-host signaling.

The extensive migratory disposition of human NSCs was especially evident at 3 and 6 months, but migratory capacity was ascertained by Dcx immunoreactivity as early as 3 weeks post-grafting. Although most HNu (+) cells remained within the gray matter, there was also some colonization of the white matter. A consistent phenomenon was the early establishment of linear pathways of migration from the initial graft into the white matter perpendicular to the spinal cord surface that often appeared to extend all the way to or close to the pia. In DAPI-counterstained preparations, it was apparent that these migratory pathways were associated with microvessels.

Neuronal differentiation of NSC grafts in the adult spinal cord has been the exception, rather than the rule, in the literature [23-26]. The relative success in the neuronal differentiation of NSCs when these cells were grafted into the developing spinal cord [69] is consistent with the idea that essential inductive signals are present in the immature but perhaps not the adult spinal cord [22, 70]. The substantial neuronal differentiation of ES/embryonic body-derived NSCs in the adult spinal cord achieved by McDonald and colleagues [27] was the first indication that the adult spinal cord environment may allow neuronal differentiation under certain conditions. The extensive neuronal differentiation of the NSC preparation used in the present study is due to several potential reasons, including species of NSC origin and culture method. Human NSCs have a more marked pluripotentiality compared to rodent NSCs [71]. In addition, NSCs used in the present study were propagated in monolayer cultures as compared to cell aggregates, i.e. neurospheres, which require harsher treatment prior to the suspension of cells for grafting. The fact that ˜5% of human NSCs express PSA-NCAM prior to grafting implies that a portion of grafted cells had already made a neuronal lineage choice. However, this pre-inoculation choice of a very small minority of grafted NSCs cannot account for the massive neuronal differentiation of the graft that is bound to have derived through multiple-step differentiation post-inoculation.

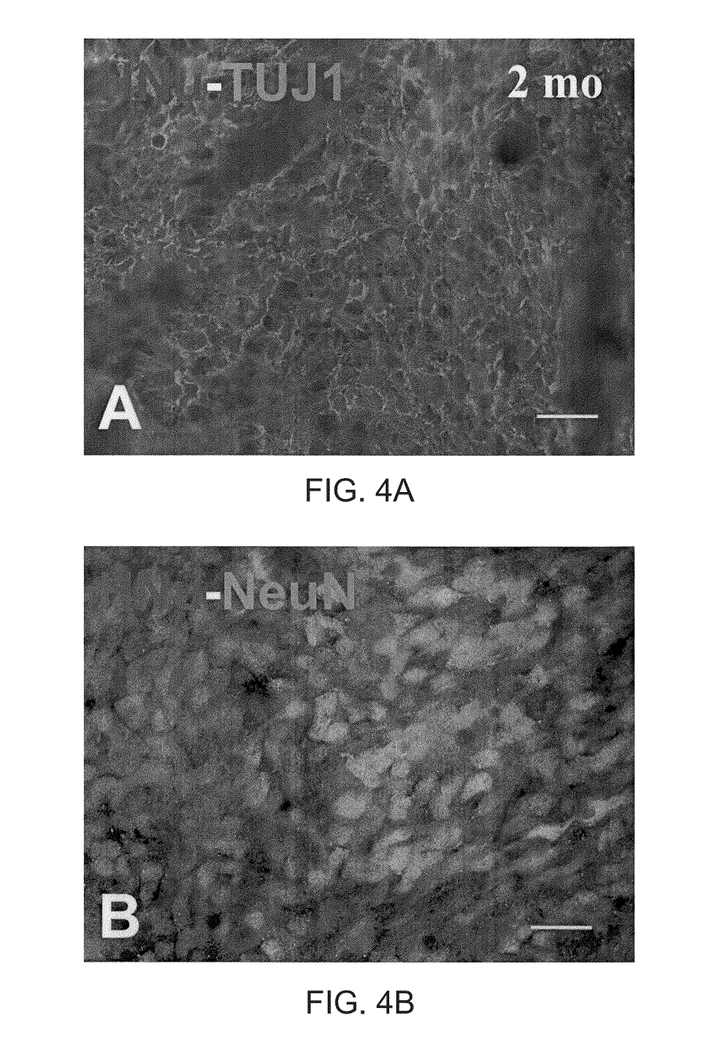

The robust differentiation observed in the present Examples amplifies and extents the findings of McDonald et al and is inconsistent with the notion that the adult cord environment is constitutively unfavorable to the survival and differentiation of NSCs [22,70]. The T-cell deficient state of the experimental subjects used here is very unlikely to have influenced the grafting outcome. In addition to the successful grafting of human NSCs in nude rats described herein, excellent survival and neuronal differentiation of human NSC grafts was observed on completely normal rats (FIG. 4A-4B) or SOD1-G93A transgenic rats and mice [72] [73] after treatment with immunosuppressive agents.

The present invention includes that local factors can influence the fate choice of grafted NSCs, i.e. many more NSCs turn into astrocytes or remain nestin (+) when located in or close to the meninges (pia). This is welcome evidence of the plasticity of NSCs and demonstrates that signals from the host microenvironment play significant roles in fate determination of grafted NSCs.

The predominant neurotransmitter phenotype of differentiated NSCs is inhibitory-GABAergic, although a measurable minority of these cells elaborates cholinergic phenotypes. These two neurotransmitter signatures appear to belong to neurons with different cytologies, i.e. GABAergic neurons have smaller bipolar cytologies whereas cholinergic neurons are larger and multipolar. However, all shapes and sizes between these two types have been encountered and, in a few cases of grafts growing outside the spinal cord parenchyma, we have seen a continuous pattern of differentiation in colonies in which GABAergic neurons predominate in the periphery and cholinergic neurons at the center. Therefore, it is unlikely that these two phenotypes belong at the end points of distinct differentiation lineages and it is possible that some GABAergic neurons could turn into cholinergic nerve cells given the proper instructive signals. Another argument against the idea of the GABAergic phenotype as an end-point fate is the fact that a majority of HNu (+) cells in the spinal cord extend long axons, whereas GABAergic spinal cord neurons in mature animals are interneurons [75,76]. In addition, many NSC-derived GABAergic neurons are islet-1 and p75^{NTR}(+), which supports the idea that at least a subset of them may be transitional motor neurons [77] stalled in their differentiation path because of a lack of target-derived differentiation signals [78,79]. The relatively higher rate of p75^{NTR} expression by GABAergic rather than cholinergic neurons is also suggestive of a single motor neuron differentiation lineage in a stage of separation from muscle targets [77]. These ideas imply that ensuring proper target connectivity for grafted NSCs in the spinal cord is not only essential for circuit restoration, but also for the proper differentiation of the graft, especially with respect to the most advanced stages of fate determination. It has been reported that “priming” type manipulations of human NSCs prior to grafting may advance them more fully to cholinergic fates post-grafting, both in the developing and adult spinal cord [80,81] but the likely precursor state of these cells and unknown survival rates and grafting efficiency does not allow direct comparisons with the results of the present study.

A substantial degree of elongation of NSC-derived axons was observed within the spinal cord parenchyma and, in many cases, along the ventral roots. It appears that there was very little, if any, inhibitory effect from the host in the elaboration and elongation of these axons. The myelin-associated glycoprotein (MAG.) and the oligodendrocyte-myelin glycoproteins NOGO A, B, and C have been shown to inhibit axonal regeneration [82], although neurotrophic factors can antagonize these effects by increasing intracellular cAMP levels [83,84]. The lack of substantial axonal growth inhibition seen in our cases may be due to species incongruence, i.e. it is possible that rodent axonal inhibitory factors cannot bind to human NOGO receptors expressed by NSCs. Other factors may have to do with developmental cycle incongruence, i.e. it is possible that young neurons from high mammals such as humans can persist in an immature state for much longer periods than rodent neurons, thereby “overwhelming” intrinsic rodent mechanisms that restrict axonal formation and elongation.

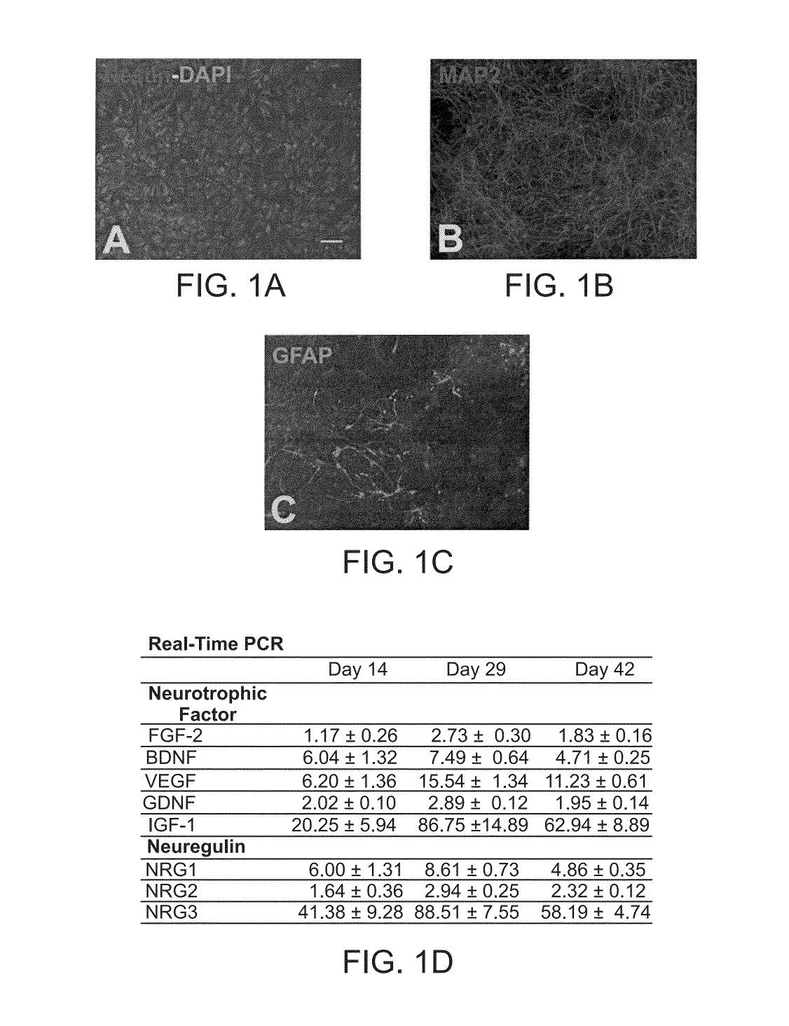

The degree of anatomical integration of grafted cells with host neurons via the establishment of reciprocal contacts is remarkable both because of its magnitude and its significant theoretical and practical implications. Although the methods of the present invention are the first to demonstrate such high levels of integration, the molecular mechanisms are still unknown. We can hypothesize that a number of trophic and cell-cell recognition transcripts shown to be expressed by cultured human NSCs play significant roles in these processes. These proteins are expressed and released in amounts and gradients that allow highly conserved tropic and trophic interactions to occur irrespective of age of the host or the fact that host and graft belong to different species. Based on our PCR data on human NSCs in culture (FIG. 1D) these protein signals include: GDNF and the neurotrophin BDNF, i.e. powerful trophic signals for motor neurons that can explain the marked ability of human NSCs to attract host motor axons [85-87]; neuregulins 1, 2 and 3, i.e. critical glial trophic signals that may also guide host axons and promote host-graft integration [88-94]; the growth hormone-like peptide IGF-1; and angiogenic factors like FGF and VEGF. Besides the neurotrophic effects of VEGF and IGF-1 [95-97], VEGF may also play a role in migration and position patterning of developing motor neurons [98]. Interestingly, IGF-1 may cooperate in tandem with BDNF to promote neuronal precursor differentiation [99], and this interaction may be particularly relevant for the findings of this study.

Thus the methods of the present invention relate to the successful grafting of NSCs from human spinal cord propagated in vitro into the adult rat spinal cord under a variety of experimental conditions and show marked differentiation into projection neurons that engage in circuit formation with the host irrespective of species differences. These outcomes indicate that, with successful suppression of host immune rejection of the graft, rodent models can be used for the preclinical evaluation of human NSCs as cell replenishment tools to a much larger extent than previously thought. It appears that cellular and molecular factors that signal circuit formation are remarkably conserved within the mammalian class, despite the fact that spatial and temporal patterning of developmental events is different among species and requires careful consideration.

Immunosuppressive Regimens Promote Graft Survival of Neural Stem Cells into the Adult Spinal Cord

In another aspect of the present invention, effective immunosuppressive regimens can be used to prevent the rejection of human NSCs grafted in SOD1-G93A mice. Our findings indicate that combinations of immunosuppressive drugs have significantly better outcomes compared to FK506 monotherapy in preventing graft rejection and, apparently via their promotion of graft survival, improving key parameters of motor neuron disease in SOD1-G93A mice. Similar results were obtained with CD4 antibodies. The effective suppression of NSC graft rejection allowed sufficient time for the differentiation of grafted cells into neurons and the establishment of networks linking host and graft neurons. Based on our data, combined immunosuppressive regimens or CD4 antibodies appeared to protect NSC grafts by suppressing CD4- and CD8-cell recruitment into the graft area and attenuating the microglial phagocytic response from surrounding spinal cord tissues. NK cells were rarely seen at the graft sites, and this is consistent with previous findings pointing to the low significance of these cells in xenograft rejection in the brain [100-101]. Within the clinical framework of our experimental design, the FK506+rapamycin combination was optimal due to its efficacy and relative simplicity.

Xenograft rejection is a serious problem when studying cell grafts of human or other highly discordant mammal origin in rodent models. For example, human spinal cord-derived progenitors are rejected 4 weeks after grafting into the adult rat spinal cord despite the use of cyclosporine A (14). In our hands, FK506, which is 10-100 times more potent than cyclosporine A in preventing graft rejection [102], has shown good results in rats grafted with human NSCs (16), but has fallen short of preventing rejection of the same cells in SOD1-G93A mice as described in the present Examples. The degree of species disparity between donor and host appears to be a key factor, and, in this respect, differences between mouse and rat may be significant. [101, 103]

Host rejection of xenografts is a complex response that is not fully understood. Although antibody-mediated mechanisms and participation of the complement have been implicated [104, 105], most evidence points to a central role for T-lymphocytes and microglia-derived macrophages [106]. The indefinite survival of human cell grafts in the nervous system of nude athymic rats observed by others [107] and by us [108] strongly supports the idea for a central role of T-cells in neural xenograft rejection. In response to foreign antigens presented by the xenograft, CD4 T-cells turn into helper cells that subsequently activate CD8 T-cells into cytotoxic roles, including the lysis of grafted cells expressing MHC class-1 epitopes on their membranes. CD4 cells also signal the transformation of CNS microglia into macrophages. Although CD8 cells are the effectors that execute the lysis of grafted cells and abound at the graft site, CD4 lymphocytes are the initiators of obligatory early signaling; for example, CD4 antibodies prevent the rejection of CNS grafts, whereas CD8 antibodies have a very weak effect. Based on our findings, CD4 antibodies are a viable alternative to immunosuppressive drugs to prevent rejection of human xeno grafts.

The use of combinations of immunosuppressants exploits some differences in their mechanisms of action without exposing animals to the side effects of extremely high doses of single compounds [102]. FK506 is a calcineurin-dependent inhibitor that blocks the production of interleukin 2 (IL-2) and thus the proliferation of T-lymphocytes. It may also have an additional role via blocking the glucocorticoid receptor. Rapamycin has inhibitory effects on IL-2 signaling downstream and independent of the calcineurin pathway and inhibits the progression of T-cells from the G to S phase. Thus, FK506 and rapamycin influence different steps in the IL-2 pathway. MMF reduces the proliferation of T-cells by blocking purine synthesis via inhibition of type II isomer of inosine monophosphate dehydrogenase. The principle of mixing these immunosuppressant compounds in various combinations is routinely used to prevent graft rejection in clinical settings [109]. However, the optimization of immunosuppressive treatments to prevent rejection of discordant neural xenografts in experimental animals has not been systematically studied. A potential concern is whether these immunosuppressive compounds can cross the blood-brain barrier (BBB). FK506 and rapamycin can readily cross the BBB, but there is no published work, nor does the manufacturer have any data, on the permeability of the BBB to MMF. However, CNS bioavailability is unlikely to influence the efficacy of these drugs whose primary mode of action is suppression of T-cell proliferation in sites outside the CNS.

Besides their prevention of xenograft rejection, immunosuppressive compounds may also exert neuroprotective effects under certain conditions [110, 111]. Cyclosporine A, FK506, and rapamycin have all shown variable efficacy in models of traumatic injury, anoxia-ischemia, and neurodegeneration. With respect to SOD1-G93A mice, only cyclosporine A has shown some efficacy [112], whereas FK506 made no difference [113]. As far as we know, rapamycin and MMF have not yet been tested. In our hands, cyclosporine A was ineffective in altering the course of motor neuron disease in SOD1-G93A mice. In addition, when FK506 and rapamycin were given in combination to mice grafted with dead human NSCs, they had no obvious effects in clinical outcomes. Therefore, in the context of the present grafting methods, the therapeutic contributions of the immunosuppressive compounds themselves to Tg motor neuron disease were negligible.

### Survival and Differentiation of Neural Stem Cells in the Degenerate Spinal Cord

In still another aspect of the present invention, NSCs can survive and differentiate into neurons in the degenerative spinal cord environment of SOD1 G93A rats. These grafts, whose immune rejection can be effectively prevented with FK-506, afford both clinical and biological benefits that are powerful and significant. The potency of this effect can be best appreciated if one considers the fact that, for a disseminated illness like ALS, lumbar cord grafting is a partial approach that omits other vital portions of the segmental motor apparatus, i.e. the cervical motor neuron column responsible for respiratory movements. However, we cannot rule out that the release of BDNF and GDNF into the CSF might have had a broader effect on host motor neurons throughout the cord. Wider grafting procedures, i.e. including both lumbar and cervical regions have significantly higher mortality, but we have had recent success with grafting sites involving both regions (L. Xu, J. Yan and V. E. Koliatsos, personal observations).

The apparent resistance of grafted NCSs to the ongoing degenerative process in the ventral horn of G93A SOD1 rats is especially important. The survival and extensive differentiation of NSCs reported here is a strong indication that the inflammatory/excitotoxic signaling involving motor neurons harboring G93A SOD1 has no evident toxicity on cells without adverse genetic properties. This factor alone raises optimism for cellular strategies using grafts to restore motor function in degenerative MND.

The magnitude of clinical and biological effects described here is in the neighborhood of effects reported in other studies on experimental therapeutics of motor neuron disease in SOD1 transgenic rodents that focus on prevention of cell death and promotion of motor neuron survival. These trials have tested a number of small organic compounds and trophic factors and have included: copper chelators [114]; compounds targeting microglial-inflammatory and glutamatergic processes [115, 116]; selective [117,118] as well as non-selective [118] cyclooxygenase-2 inhibitors alone or in combinations with other compounds; GDNF retrogradely delivered to motor neurons from muscle with adeno-associated virus or via motor neuron transfer with lentivirus [119-121]; and VEGF transferred to motor neurons via lentiviral vector injected into various muscles [122] or via direct delivery [123-124]. With the exception of a recent study using intraventricular VEGF [124], the vast majority of these studies have involved SOD1 Tg mice [125-126] in which MND tends to be of later onset and milder progression than in the SOD1 Tg rats used here. The only other example of NSC treatment of SOD1 Tg rodents known to us is a study in which investigators grafted GDNF-engineered, cortically derived human neural progenitors in the spinal cord of G93A SOD1 rats and found that these cells survived well and expressed high levels of immunoreactive GDNF; most of these cells remained at a nestin (+) precursor state and showed no apparent structural integration or clinical benefits.

The Examples described herein identify some cellular and molecular mechanisms mediating the therapeutic effects of NSCs in Tg MND. Although NSCs engage in reciprocal connections with host motor neurons, they do not appear to exert therapeutic effects by replacing degenerated neuromuscular units. Based on our findings on the expression and release of trophic peptides, at least a portion of the effects of NSCs on degenerating motor neurons may be their delivery, via classical mechanisms including transsynaptic transfer, of neurotrophins and especially trophic cytokines to degenerating host motor neurons. In other words, the surprising establishment of synaptic appositions between grafted human NSCs and rat motor neurons, also observed in a recent study between human NSC grafts and mouse motor neurons [127], may play a significant therapeutic role beyond the obvious advantage of strengthening/amplifying local circuits. The significance of NSC-derived GDNF for the survival of motor neurons has also been stressed in another in vitro study showing a protective effect of this trophic cytokine on rat spinal cord explants exposed to an excitotoxic protocol [128].

### Transplantation

Cell transplantation into patients can be achieved, for example, by filling a syringe with cells to be transplanted, suspended in artificial cerebrospinal fluid, physiological saline or the like, exposing damaged neural tissue by operation, and directly injecting the cells into the damaged area with an injection needle. The cells of the present invention can then migrate into neural tissues due to their high migration activity. Therefore, the cells may be transplanted into a portion adjacent to a damaged area. Injection of the cells into the cerebrospinal fluid may also be effective. In this case, the cells can be injected by typical lumbar puncture, and thus is preferable, since the patient is treated with only local anesthesia and without operation in a sickroom. Moreover, intraarterial injection and intravenous injection can also be effective, and hence the transplantation can be practiced by the same procedure as typical blood transfusion.

The methods of the present invention can be transplanted into recipients for the purpose of treating neurological diseases. The neurological diseases to be treated include, but are not limited to, demyelination diseases in the central and peripheral nervous systems, degenerative diseases in the central and peripheral nervous systems, spinal cord tumors, brain dysfunction including traumatic neurological diseases (including spinal cord injury), inflammatory diseases, infectious diseases (for example, Creutzfeldt-Jakob disease) and infarction of spinal cord.

All publications and patent applications cited in this specification are herein incorporated by reference as if each individual publication or patent application were specifically and individually indicated to be incorporated by reference.

### Exemplification

Materials and Methods for Examples 1 thru 6

### Derivation of Human NSCs

Human NSCs were prepared from the cervical and upper thoracic spinal cord of a single 8-week human fetus after an elective abortion. The tissue was donated by the mother in a manner fully compliant with the guidelines of NIH and FDA and approved by an outside independent review board. Spinal cord tissue was cleared of meninges and dorsal root ganglia and dissociated into a single-cell suspension by mechanical trituration in serum-free, modified N2 medium composed of 100 mg/L human plasma apo-transferrin, 25 mg/L recombinant human insulin, 1.56 g/L glucose, 20 nM progesterone, 100 μM putrescine and 30 nM sodium selenite in DMEM/F12, to which basic fibroblast growth factor (bFGF) (10 ng/ml) was added. The initial culture was serially expanded as monolayer in pre-coated flasks or plates. Approximately 6.1×10⁶ cells were obtained upon the initial dissociation of the spinal cord tissue. All cells were plated on to one 150 mm plate in 20 ml of growth media. Growth medium was changed every other day and, on alternate days, 10 ng/ml of bFGF was added. The first passage was conducted at 16 days post-plating. At this time point, the culture was composed mostly of dividing NSCs and post-mitotic neurons. Dividing cells were harvested by brief treatment with trypsin (0.05%+0.53 mM EDTA) followed by mechanical trituration. A single cell suspension was thus derived that was centrifuged at 1400 rpm for 5 minutes. The cell pellet was resuspended in the growth medium and cells were replated in new pre-coated plates at 1.2×10⁶ cells in 20 ml of medium per 150 mm plate. Cells were harvested at approximately 75% confluence, which occurred within 5 or 6 days. This process was repeated for 20 passages. Cells from various passages were frozen in the growth medium plus 10% DMSO and stored in liquid nitrogen. Upon thawing, overall rate of recovery was 80-95%. The resulting cell line, produced by epigenetic means only and by using bFGF as the sole mitogen, was coded “566RSC”.

Passage 10-12 cells were used in this study. Five to seven days prior to surgery, one cryopreserved vial of the appropriate passage was thawed, washed, and cultured again as described above. For multiple days of surgeries, cultures were seeded at varying densities so that each flask reached confluence on the designated day of surgery. Cells were subsequently harvested by brief enzymatic treatment as described above, washed in a buffered saline solution, couriered to the surgery site on wet ice, and used within 24 hours. Viability of cells on ice was typically greater than 80% within this 24-hour period.

### Experimental Subjects and Surgical Procedures

Nude rats (160-180 g, strain CR: NIH-RNU, n=37) were purchased from the animal service of the National Cancer Institute and were the main experimental subjects of this study. An additional small number of commonly used Sprague-Dawley rats (n=3, Charles River Laboratories, Wilmington, Mass.) was used for the purpose of proving the concept that the results reported here were unrelated to the athymic state of our experimental subjects. Normal Sprague-Dawley rats were immunosuppressed with daily injections of FK506 (2 μg/g i.p.; Prograf, Fujisawa Healthcare, Inc., Deerfield, Ill.). Animal surveillance and the surgical procedures described here were carried out according to protocols approved by the Animal Care and Use Committee of the Johns Hopkins Medical Institutions. Surgeries were performed using gas anesthesia (enflurane:oxygen:nitrous oxide=1:33:66) and aseptic methods. All nude rats were subjected to surgical interventions two weeks prior to NSC grafting; interventions included proximal ventral rhizotomies [34](n=18), excitotoxic lesions [13](n=12) or sham surgeries (n=6). Rats of normal immunological constitution were not subjected to lesions prior to grafting.

Rhizotomies involved transections of L4 and L5 roots with extraspinal avulsion of the corresponding spinal nerves as described [34]. In brief, the left L4 & L5 spinal nerve roots were exposed at the level of the iliac crest after splitting the sacroiliac joint and roots were avulsed by applying a steady moderate traction with forceps. Excitotoxic lesions were made with 100 μl of 150 mM of the kainate analog L-homocysteic acid (HCA) [35] absorbed into a gelfoam pledget that was laid on the dorsal dura matter of L4-L5 for a period of 20 minutes after a dorsal laminectomy. This strategy optimizes the need to lesion a rather extensive group of vulnerable (motor) neurons throughout L4-L5 without causing epileptogenesis. The sham group was subjected to dorsal laminectomy and subsequent wound closure. The parallel use of axotomy and neurotoxic lesions allowed some variance in the method of inducing motor neuron death, and the neurotoxic paradigm offered the additional advantage of preserving the conduit for the assessment of de novo axonal elongation towards the periphery.

Two weeks after lesion, animals were subjected to dorsal laminectomy at the lower thoracic level and received 4 injections of 10⁵ NSCs in 0.5 μl suspension into ventral L4 and L5 on the left side. Injections were made streotaxically on a Kopf spinal unit 1 mm lateral to the midline using pulled-beveled glass micro-pipettes connected, via silastic tubing, to 10 μl Hamilton microsyringes. Animals were allowed to survive for 3 weeks, 3 months, or 6 months (avulsion lesions), 3 or 6 months (excitotoxic lesions), or 6 months (sham lesions) (n=6 for each treatment×time point).

### Histology, Immunocytochemistry (ICC) and Microscopy

The survival and phenotypic fate of NSCs were assessed with ICC, including ABC-peroxidase ICC and dual-label immunofluorescence. Tissues were prepared from animals perfused with 4% freshly depolymerized, neutral-buffered paraformaldehyde. The thoraco-lumbar spinal cord segments with attached roots and lumbar nerves were further fixed by immersion in the same fixative for an additional 4 hr after removal of the dura. Blocks containing the entire grafted area plus 1 mm border above and below were subdissected, equilibrated in 30% neutral-buffered sucrose, and frozen for further processing. L3-S1 roots were processed separately as whole-mount preparations or after teasing the rootlets with heat-coagulated tips of glass pipettes. Blocks were sectioned transversely (30 μm) on a freezing microtome; sections were kept in an antifreeze solution until processed for NSC survival or phenotypic studies. Survival studies utilized human nuclear antigen (HNu) immunoperoxidase-stained sections (˜15 per animal, i.e. every 24^{th} section through the L3-S1 block) with random sampling of the first section. HNu is a selective nuclear marker of cells of human origin [36]. NSC differentiation utilized 4-5 sections 1.5 mm apart taken through the grafting area and stained with dual-label immunofluorescence; in most cases, dual-label immunofluorescence combined HNu with another cellular marker.

After permeabilization with 0.1% Triton X-100 and non-specific site blocking with 5% normal serum, slide-mounted sections were incubated in primary antibodies in 1 mg/ml BSA with 0.1% Triton X-100 (4° C., overnight). Primary antibodies were used to address human (graft) versus rat (host) cell identity, mitotic activity, and neuronal, astrocytic, and oligodendrocytic phenotype specification and included a number of monoclonal antibodies and antisera as laid out in Table 1. Normal IgG from the species of origin of the primary antibodies served as negative controls. Peroxidase-based detection of HNu immunoreactivity utilized an enhanced version of the avidin-biotin method (ABC-elite kit; Vector, Burlingame, Calif.) and a standard DAB chromagen reaction. Dual immunofluorescence utilized an indirect protocol combining goat/donkey anti-mouse IgG and goat/donkey anti-rabbit IgG labeled with Cy3 or Cy2 in a corresponding fashion (1:200; Jackson ImmunoResearch, West Grove, Pa.); anti-rabbit IgGs were used against the species of origin of the phenotype-marking antibody. Sections were incubated in these antibodies for 2-4 hours at RT and were then counterstained with the fluorescent DNA dye 4′,6-diamidino-2-phenylindole (DAPI). Sections were mounted with mounting medium DPX and studied with epifluorescence on an Axiophot Zeiss microscope or with confocal microscopy on a Zeiss LSM 410 unit. Triple immunofluorescence was used for the colocalization of HNu, Bassoon, and TUJ1. This procedure involved first a dual (indirect) immunofluorescent step in which Cy5 replaced Cy3 as fluorophore for the goat anti-mouse IgG, and then a direct immunofluorescent step in which sections were incubated in HNu antibody coupled to the red fluorophore Alexia-594 (Molecular Probes, Eugene, Oreg.).

### Cell Counts for Surviving and Differentiating NSCs

To assess NSC graft survival, we counted total numbers of HNu (+) profiles through L4-L5 on immunoperoxidase-labeled sections based on stereological assumptions. The optical fractionator probe [37] was applied with the aid of a motorized-stage Axioplan microscope equipped with the Stereo Investigator V hardware and software (MicroBrightField Inc., Williston, Vt.). As explained in the previous section, sections had been sampled in a randomized systematic fashion. Corrected section thickness after processing was 18 μm.

The contour of each half of spinal cord section was outlined at 5× by a blinded investigator and cells were counted using a 100× oil-immersion objective. A 40×35 μm counting frame was used within a 300×300 μm grid coextensive with the outlined area; the counting depth (optical dissector height) ranged 9-13 μm according to the average section thickness per case. A guard volume of 1.0 μm was used during cell counts to avoid sectioning artifacts, including lost caps and uneven section surfaces.

To study NSC differentiation, we used a non-stereological method of counting total number of HNu (+) cells, as well as cells dually labeled with HNu and a phenotypic marker on randomly selected high-power (100×) fields from immunofluorescent preparations. One field in each one of 4 sections, spaced 1 mm apart through the grafting area, was used from each animal. Numbers of HNu (+) and double-labeled profiles were pooled from all 4 fields counted from each case and grouped per experimental protocol. Average numbers of single and double-labeled cells were generated for each treatment group (n=4 per group).

Variation in survival and differentiation as function of experimental protocol, graft location, and time point post-surgery was studied with one-way ANOVA followed by Tukey's Multiple Comparison post hoc test, except in cases where only two groups were tested, where students' t test was more appropriate. Results are expressed as mean±SD.

### Real-Time PCR Analysis of the Expression of Relevant Trophic and Tropic Genes

RNA samples were extracted from 10⁷ NSCs at various time points of differentiation in vitro (0, 14, 29, and 42 days after withdrawal of bFGF) using Trizol (Invitrogen, Carlsbad, Calif.). Residual DNA was removed using the DNA-free kit (Ambion, Austin, Tex.). Template cDNA was synthesized from 1 μg of RNA with the iScript cDNA Synthesis Kit (Bio-Rad, Hercules, Calif.) and was diluted 10-fold before PCR amplification. SYBR green-based real-time PCR was performed using the iCycler (Bio-Rad). 18s rRNA was used as the reference transcript (e.g. control gene). Table 2 lists the human-specific primers for bFGF [38], brain-derived neurotrophic factor (BDNF) [39], vascular endothelial growth factor (VEGF) [38], glial cell line-derived neurotrophic factor (GDNF) [40], insulin-like growth factor-1 (IGF-1) [41], and neuregulins 1, 2, 3 (NRG1, 2, and 3) [42], all of which were assayed because of suspected or established role in motor neuron development. PCR reactions, run in triplicate for each sample, were carried out in 25 μl volume containing 2 μl of 1:10 dilution of cDNA, 0.5 μl of each sense and antisense 10 μM primer stocks, 12.5 μl iQ SYBR green Supermix (Bio-Rad), and nuclease-free water. For either 18s rRNA or mRNA of interest, except neuregulin transcripts, cycling conditions were 95° C. for 5 min, followed by 40 cycles of 95° C. for 15 sec and 60° C. for 1 min. For NRG1, 2, and 3 the annealing temperature was 56° C. Reactions without template served as negative controls. Melting curve analysis was carried out by heating the amplicon from 50 to 95° C. in 90 0.5° C. increments. PCR efficiency curves were made for each gene using 5 duplicate 5-fold dilutions, corresponding to 0.08 to 50 ng of the initial total RNA input into the RT reaction. Data was analyzed using the Bio-Rad Gene Expression Macro version 1.1 for Microsoft Excel. Normalized gene expression ratios were calculated as described [43]. Briefly, PCR efficiencies were calculated according to the equation E=e^{[-1/slope]}. The relative expression ratio of target genes was calculated using the equation: ratio=(E_{target})^{Δ target Ct (control-sample)}/(E_{ref})^{Δ} reference Ct (control-sample), where the Ct is the point at which SYBR green fluorescence rises above background fluorescence. Gene expression levels from Day 0 cDNA were designated as 1, to which other time points were compared. Standard deviations for the relative and normalized expression values were calculated.

### Materials and Methods for Examples 7 Thru 9

### SOD1 G93A Breeding

SOD1 G93A male rats supplied by Dr. David S. Howland, University of Washington, Seattle, were bred to 4 Tac: N(SD) female rats from Taconic (Germantown, N.Y.). Rats were bred for one week in pairs of one male: one female Offspring were weaned and genotyped at 21 days of age and positive transgenic pups were identified for treatment. Colony was propagated by back-breeding male pups of the same litter with the original female breeders to reduce phenotypic variance.

### Derivation and Propagation of Human NSC's

Human NSCs were prepared from the cervical-upper thoracic cord of a single 8-week human fetus after an elective abortion. Tissues were donated by the mother in a manner fully compliant with the guidelines of NIH and FDA. Spinal cord tissues cleared of meninges and dorsal root ganglia were dissociated into a single cell suspension by mechanical dissociation in serum-free, modified N2 medium and serially expanded in monolayer [44]. Growth medium was changed every other day, and, on alternate days, 10 ng/ml of bFGF was added to the culture. The first passage was conducted at 16 days post-plating, a time point at which the culture was composed mostly of dividing NSCs and post-mitotic neurons. Dividing cells were harvested by brief treatment with trypsin followed by dissociation and replated in new pre-coated plates. Cells were harvested at ˜75% confluence, which occurred within 5 or 6 days. This process was repeated for up to 20 passages. Cells from various passages were frozen in the growth medium plus 10% DMSO in liquid nitrogen. Upon thawing, recovery rate was 80-95%.

Passage 10-12 cells were used in this study. Five to seven days prior to surgery, one cryopreserved vial of the appropriate passage was thawed, washed, and cultured again. Cells were harvested by brief enzymatic treatment as described above, washed in buffered saline, couriered to the surgery site on wet ice, and used within 24 hours. Viability of cells on ice was typically greater than 80% within this 24-hour period. Immediately prior to grafting, the overwhelming majority of human NSCs expressed nestin, whereas ˜5% were immunoreactive for PSA-NCAM. Less than 1% expressed the neuronal markers TUJ1 and MAP2 or the astroglial marker GFAP.

### Surgical Procedures

All surgical procedures were carried out according to protocols approved by the Animal Care and Use Committee of the Johns Hopkins Medical Institutions using gas anesthesia (enflurane:oxygen:nitrous oxide=1:33:66) and aseptic methods.

Live or dead NSCs were grafted into the lumbar protuberance (L4 & L5) of 62 day-old SOD1 G93A rats (220-300 g; mixed gender; n=45) mounted on a Kopf spinal stereotaxic unit under microscopic guidance. Twenty seven subjects grafted with live (n=16) or dead (n=11) cells were used in motor testing followed by anatomical analyses. The remaining animals (n=18) were used for motor neuron survival, ELISA, Western Blot, and real time RT-PCR studies equally divided between subjects grafted with live and subjects grafted with dead cells.

Dead cells were prepared by 3× freezing in liquid nitrogen (−70° F.) and then thawing in room temperature. To confirm cell death of NSCs, 10 μl of the cell suspension was mixed with 40 μl of Hank's balanced salt solution and 50 μl of stock Trypan Blue solution was added to the mixture. Solution was left for 5 min at room temperature and then a drop of it was placed on a hemacytometer and studied with light microscopy. All cells prepared as above were seen to take up the Trypan Blue stain, evidence that they were destroyed.

Cell suspensions were delivered under aseptic conditions via 8 injections aimed at the ventral horn on both sides (1 μl with 5×10⁴ NSC per injection site, 4 injection sites per side) with pulled-beveled glass micro-pipettes connected to 10 μl Hamilton microsyringes via silastic tubing. All rats received FK-506 (1 mg/kg i.p. daily) till euthenized to prevent immune rejection. Rats were killed with perfusion-fixation when their BBB score (see below) was <3.

### Animal Testing—Clinical Outcomes

Rats were tested for motor strength and weight twice weekly. Motor strength tests included the BBB locomotor rating scale [45, 46] and the inclined plane scale [47]. For BBB scoring, animals were tested for 4-5 minutes in an open field. For inclined plane scoring, rats were placed on the inclined mat and plane angle was adjusted to the highest point at which the animal could retain position for 5 seconds; this angle was then recorded as the subject's inclined plane score. BBB and inclined plane scores were analyzed by repeated-measures ANOVA followed by Fisher LSD post hoc test to assess differences between live- and dead-cell groups. Disease onset was defined as the point at which body weight was found decreased for the second consecutive time, i.e. a sensitive and very objective measure for determining disease onset in murine models of ALS [48]. Course of illness as an effect of graft type (live- or dead-cell graft) was analyzed by comparing age at disease onset and age of death between the two groups (with student's t test) as well as with Kaplan-Meier survival analysis followed by log-rank testing.

### Histology, ICC, and Microscopy

Tissues were prepared from animals perfused with 4% freshly depolymerized, neutral-buffered paraformaldehyde based on protocols approved by the Animal Care and Use Committee of the Johns Hopkins Medical Institutions. The thoraco-lumbar spinal cord segments with attached roots and lumbar nerves were further fixed by immersion in the same fixative for an additional 4 hr after removing the dura. Blocks containing the entire grafted area plus 1 mm border above and below were cryoprotected and frozen for further processing. L3-S1 roots were processed separately as whole-mount preparations or after separating rootlets with heat-coagulated tips of glass pipettes. Blocks were sectioned (35 μm) at the transverse or saggital plane.

NSC survival and differentiation was studied with dual-label immunofluorescence that combined, in most cases, HNu with another cellular marker and was performed essentially as described [17]. Primary antibodies were from Chemicon International (Temecula, Calif.) except otherwise noted and included: mouse anti-HNu protein antibody (1:800); rabbit anti-TUJ1 (1:400; Research Diagnostics Inc., Flanders, N.J.); rabbit anti-GFAP (1:400; Dako Carpinteria, Calif.); rabbit anti-human nestin (1:200); mouse anti-NF70 (1:100, human and porcine specific); goat anti-human GDNF (1:400, R&D Systems Inc. MN); mouse anti-SYN (1:200, human and hamster specific); mouse anti-BSN (1:400, rat and mouse specific; Stressgen, Victoria, BC); goat anti-ChAT IgG (1:100); Guinea pig anti-VGLUT 1/2 (1:10,000, rat specific); and guinea pig anti-VAChT (1:500). Dual immunofluorescence utilized an indirect protocol combining donkey anti-mouse and donkey anti-rabbit or donkey anti-goat IgG labeled with Cy3 or Cy2, respectively (1:200; Jackson ImmunoResearch, West Grove, Pa.). Sections were incubated in linking antibodies for 2-4 hours at room temperature and were then counterstained with the fluorescent DNA dye DAPI, coverslipped with DPX, and studied with epifluorescence or confocal microscopy. Triple immunofluorescence was used for the colocalization of HNu, Bassoon, and TUJ1; this procedure involved first a dual immunofluorescence step with donkey anti-rabbit IgG-Cy2 and donkey anti-mouse IgG-Cy3 and then an additional incubation in biotinylated HNu antibody followed by treatment with streptavidin-AMCA (Jackson). Digital images were optimized for brightness/contrast and resolution (600 ppi) with the aid of Adobe PhotoShop 6.0 software (Adobe Systems, San Jose, Calif.).

### Cell Counts for Differentiated NSCs and Stereological Counts for Surviving α-Motor Neurons Motor Neurons

To study NSC differentiation, we used a non-stereological method of counting the total number of HNu (+) cells, as well as cells dually labeled with HNu and a protein marker of neural cell differentiation on randomly selected high-power (100×) fields from our immunofluorescent preparations. One field in each of 6 sections spaced ˜1 mm apart through the graft area was used from each animal. Numbers of HNu (+) and double-labeled profiles were pooled from all 6 fields counted from each case and grouped per experimental protocol. Average numbers of single and double-labeled cells were generated for the dead-or live-cell group (n=6 per group).

To assess motor neuron survival in rats grafted with live or dead cells (n=3 per group), we used tissues from animals sacrificed at 128 days of age, i.e. a mid-point in the course of disease for subjects that received dead-cell grafts. Every sixth section in the L3-S1 region from each animal was sampled as per stereological requirements [17] and stained with cresyl violet (Niss1 staining) for motor neuron counting. α-motor neurons, identified as multipolar cells with distinct nucleus and a soma diameter >35 μm [49], were counted with the optical fractionator probe using the Stereo Investigator V hardware and software (MicroBrightField Inc., Williston, Vt.) as described [17]. Differences between animals grafted with live versus dead cells were analyzed with a student's t test.

### ELISA for Motor Neurotrophic Factors: GDNF and BDNF

CSF was sampled from the fourth ventricle of animals (n=3 per dead-or live-cell group) under gas anesthesia. Animals were then euthenized by decapitation and tissue samples containing grafting sites and areas adjacent to those were dissected from transverse 1 mm-thick spinal cord slices. CSF or cord samples were processed as described [50] and levels of GDNF and BDNF protein were measured with the E-Max ImmunoAssay system (Promega, Madison, Wis.). Reaction product was revealed with HRP-conjugated chicken IgY antibody and TMB as chromogen. TMB absorbance was read with a ThermoMax microplate reader at 450 nm. Variance in concentrations among samples from live-cell grafts, areas adjacent to grafts and dead-cell grafts was analyzed with one-way ANOVA followed by a Tukey's Multiple Comparison post hoc test. Difference in CFS concentration of GDNF and BDNF between animals grafted with live versus dead cells was analyzed with student's t test.

### Western Blotting for GDNF and BDNF

Protein samples from CSF or spinal cord prepared as for ELISA were electrophoresed on 12% NuPAGE precast gels (Invitrogen, Carlsbad, Calif.) and transferred on to nitrocellulose membranes (BA-S85; Schleicher & Schuell, Keene, N.H.). Blots were blocked in TBS (pH 7.4) containing 5% donkey serum, and then incubated in GDNF and BDNF antibodies (1:500; overnight, 4° C.), followed by HRP-linked donkey anti-goat IgG (for GDNF) and anti-rabbit IgG (for BDNF) (1:2000; Jackson) (1 hr, room temperature). All antibodies were diluted in TBS containing 5% donkey serum. Blots were developed with the SuperSignal Chemiluminescent Substrate (Pierce) and exposed to Kodak-XAR film (Eastman Kodak, Rochester, N.Y.). Blots were then striped and re-blotted with β-actin antibody (1:500, Sigma), followed by HRP-linked donkey anti-mouse IgG (1:10000, Jackson). Immunoreactive bands were analyzed with Bio-Rad Quantity One software (Bio-Rad Laboratories, Hercules, Calif.). Band density ratios (GDNF or BDNF: β-actin) were calculated per animal and group means were entered for statistical analysis as in the case of ELISA experiments.

Real time RT-PCR for BDNF, GDNF, and VEGF

Cultured NSCs prior to grafting and homogenized spinal cord tissues (experimental n=4; control n=3) were subjected to RNA extraction as described [17]. Template cDNA was reverse transcribed with the iScript cDNA synthesis kit (Bio-Rad, Hercules, Calif.) and SYBR green-based real-time PCR was performed with the iCycler (Bio-Rad). Human- and rat-specific primers were synthesized by Integrated DNA Technologies (Coralville, Iowa). SDHA and RPL13a served, respectively, as the human- and rat-specific reference sequences used for normalization. PCR reactions, run in triplicate for each sample, contained diluted cDNA (1:1; 1 μl), 10 μM of sense and antisense primer stocks (0.5 μl each), iQ SYBR green Supermix (Bio-Rad) (12.5 μl) and of nuclease-free water (10.5 μl) in 25 μl volume. PCR cycling conditions were 95° C. for 5 min, 35 cycles of 95° C. for 30 sec, 60° C. for 30 sec, and 72° C. for 30 sec. Reactions without template served as negative controls. Reactions without RT containing 25 ng RNA in 1 μl served as RT (−) controls. Melting curve analysis was carried out by heating the amplicon from 60 to 95° C. in 71 0.5° C. increments. Efficiency curves were created for each amplicon using 5 duplicate 2-fold dilutions, corresponding to 6.25-100 ng of the initial total RNA input into the RT reaction. Data were analyzed using the Bio-Rad Gene Expression Macro version 1.1 for Microsoft Excel. Normalized gene expression ratios were calculated as described [51].

Levels of rat- and human-specific gene expression in spinal cords with live grafts were expressed as fold changes from baseline expression in spinal cords with dead NSCs and from live NSCs prior to grafting. Standard deviations for normalized expression values were calculated as described [52].

### Materials and Methods for Examples 10 Thru 12

### Experimental Subjects and Design

Heterozygous male B6SJL-TgN (SOD1-G93A) 1Gur mice were purchased from Jackson Laboratories (Bar Harbor, Me.) and mated with wild-type females. Animal care and surgical procedures were carried out according to protocols approved by the Animal Care and Use Committee of the Johns Hopkins Medical Institutions. Heterozygous offspring of mixed gender were selected by genotyping at ˜1 month of age.

Because, in pilot studies, we had failed to achieve acceptable human NSC graft survival with routine cyclosporin immunosuppression, the present study was designed with emphasis on alternative immunosuppressive regimens. Based on evidence for a strong T-cell-mediated rejection in pilot studies, our goal here was to use a potent combination of T-cell activation and proliferation blockers, capitalizing on commercially available calcineurin-dependent inhibitors (FK506, tacrolimus), non-calcineurin-mediated inhibitors (rapamycin, sirolimus), and inosine monophosphate dehydrogenase inhibitors (mycophenolate esters such as mycophenolate mofetil, MMF). Thus, five experimental groups were given different immunosuppressive treatments: the first group was treated with FK506 monotherapy (Prograf, Fujisawa Healthcare, Inc., Deerfield, Ill.) (2 μg/g/day, n=23); a second group was treated with a double immunosuppressive regimen combining FK506 and rapamycin (Calbiochem, La Jolla, Calif.) (1 μg/g/day each, n=21); a third group received triple immunosuppression with FK506, rapamycin, and MMF (CellCept, Roche, Nutley, N.J.) (1 μg/g/day each for FK506 and rapamycin, 100 μg/g/day for MMF, n=14); a fourth group was treated with CD4 antibodies (clone GK1.5, 20 μg/g/day, n=12); and a fifth group (n=10) was treated with FK506 plus rapamycin exactly as the second group, except that subjects were grafted with dead cells. Dead-cell grafts utilized cells that were exposed to repeated freezing-thawing and served to control for the potential therapeutic effects of immunosuppressants themselves on motor neuron disease. The FK506-rapamycin combination was the only treatment given to animals with dead-cell grafts because it is the simplest immunosuppressant regimen that protects grafts from rejection (see Results section). Males and females were randomly admixed in the various experimental groups to minimize a systemic effect of gender on disease progression and treatment response.

A group of animals was euthenized at 1 week (FK506 monotherapy, n=6; FK506 plus rapamycin treatment, n=5; FK506 plus rapamycin plus MMF, n=5; CD4 antibodies, n=4) and another group was euthenized at 1 month (FK506, n=5; FK506 plus rapamycin, n=5; FK506 plus rapamycin plus MMF, n=4; CD4 antibodies, n=3) post-grafting, to monitor host immune response to grafted cells and graft survival. All others (FK506, n=12; FK506 plus rapamycin, n=11; FK506 plus rapamycin plus MMF, n=5; CD4 antibodies, n=5; FK506 plus rapamycin in animals grafted with dead cells, n=10) were allowed to survive to end-stage illness (as defined in the Motor scoring section of Materials and Methods; typically >2 months post-grafting) in order for us to assess clinical outcomes as well as long-term graft survival and host responses.

### Derivation and Culture of Human NSCs

Human NSCs were prepared from the cervical spinal cord of a single 8-week human fetus donated by the mother in a manner compliant with the guidelines of NIH and FDA and approved by an outside independent review board. All JHMI institutional guidelines were followed in obtaining and using these cells in our laboratory. The initial culture was expanded as monolayer in poly-D-lysine and fibronectin-coated dishes using serum-free medium containing FGF-2 as described [60]. The resulting cell line, termed “566RSC”, was passaged 10-12 times prior to grafting. Five to seven days prior to surgery, one cryopreserved vial of cells was thawed, washed, and cultured again as monolayer. Cultures were seeded so as to reach confluence on the day of surgery. Cells were subsequently harvested by brief enzymatic treatment that deactivated FGF-2, washed in buffered saline, and used within 24 hours. As verified by Trypan Blue exclusion, viability of cells on ice was typically greater than 80% within this 24 hour period.

### Preparation of Monoclonal Antibody Against Mouse CD4

The monoclonal antibody GK1.5, a rat IgG2b directed against a surface epitope of mouse CD4 cells, has excellent in vivo efficacy in multiple models of T-cell-mediated rejection [29]. The antibody was prepared from GK1.5 hybridoma cells (a gift from Dr. William Baldwin, Department of Pathology, JHMI) and purified from cell culture supernatant or ascites fluid with a protein G column. For cell culture, cells were plated in plastic flasks containing Hybridoma-SFM medium with 1% ultra-low IgG fetal bovine serum and penicillin/streptomycin (all from Invitrogen, Carlsbad, Calif.) in a humidified cell culture incubator with CO₂: ambient air=5:95 (37° C.). For ascites preparation, two month-old male nude mice with the same strain background as SOD1-G93A (B6.Cg-Foxn 1^{nu} from Jackson Labs, Bar Harbor, Me.) were primed by injecting 0.5 ml of Freund's incomplete adjuvant one week before the injection of 1×10⁶ hybridoma cells in 0.5 ml PBS i.p. Ascites fluid was collected two weeks later with multiple taps.

Conditioned medium or ascites fluid were centrifuged at 2000 g. Undiluted medium supernatant or ascites supernatant diluted 1:10 in 20 mM phosphate buffer (PB, pH 7.4) were loaded onto a 5 ml Hi-trap protein G column (Amersham Biosciences, Piscataway, N.J.). The column was washed with 20 mM phosphate buffer (PB, pH 7.4) and bound antibody was eluted with 0.1 M glycine-HCl (pH 2.9) and immediately neutralized with 1M Tris-HCl (pH 9).

### Surgical Procedures

Surgeries were carried out under gas anesthesia (enflurane:oxygen:nitrous oxide=1:33:66) and aseptic conditions, with the aid of a Zeiss surgical microscope and a Kopf spinal stereotaxic unit fitted with a mouse mouth-and-nose device. NSCs were grafted via a dorsal laminectomy window into the ventral gray matter of the lower lumbar protuberance of 8-week old SOD1 G93A mice. To achieve a good correspondence with L4-L5, injections were targeted to the portion of spinal cord immediately underneath the T12 vertebra. Cell suspensions were delivered under aseptic conditions via 4 injections aimed at ventral horn (2×10⁴ NSCs in 0.5 μl per injection site, 2 injection sites on each side of the spinal cord 1 mm apart) with pulled, beveled glass micro-pipettes connected to 10 μl Hamilton microsyringes via silastic tubing.

Animals were treated with four different immunosuppressive regimens as outlined in the “Experimental subjects and design section” of Materials and Methods. Immunosuppressant compounds were given with daily i.p. injections beginning one day prior to grafting and ending on the day prior to euthanasia. To prepare FK506 for monotherapy, the commercially available FK506 solution (5 mg/ml) was diluted to 1 mg/ml with sterile distilled water. When FK506 was combined with rapamycin, 1 mg of commercially available rapamycin powder was first dissolved in 200 μl of DMSO and then combined with 200 μl of the commercial FK506 solution (5 mg/ml); the mixture was then diluted to a final 2 ml volume with the addition of 1.6 ml of sterile distilled water. For triple immunosuppression with FK506, rapamycin, and MMF, rapamycin was mixed with FK506 first, and the dilution step was done with 1.6 ml of sterile distilled water in which 100 mg of MMF had been previously dissolved. GK1.5 antibodies (diluted 10 mg/ml in sterile PBS) were injected for a total of 9 days beginning one day prior to grafting and then for 5 consecutive days every 4 weeks until sacrifice [29].

### Motor Scoring and Other Tests for Clinical Outcomes

Animals were weighed twice a week, and disease onset was defined as the time point when body weight started to decrease consecutively [61]. Motor strength testing was used to monitor disease progression and to determine terminal illness. Terminal illness, defined in greater detail below, was used as an endpoint for these experiments, according to the guidelines of the Animal Care and Use Committee of JHMI. The Basso, Beattie, and Bresnahan (BBB) locomotor rating scale [62] that is commonly used to assess motor strength places emphasis on hindlimbs. In SOD1-G93A mice, motor deficits appear first in hindlimbs but subsequently spread to forelimbs. To appropriately address the four-limb involvement and accommodate for the small animal size, we adjusted a 5-point scale originally proposed by Wrathall [63], primarily in order to distinguish between hindlimb and forelimb strength. The modified Wrathall scale scores were defined as follows: 5, normal gait; 4, mild deficits: hindlimbs lose some weight bearing; 3, moderate deficits: hindlimbs lose most weight bearing and toe clearance; 2, severe deficits: hindlimbs lose weight bearing almost completely and forelimbs begin to show signs of weakness; 1, end-stage disease: hindlimbs are completely paralyzed, forelimbs are affected to various degrees, and the mouse cannot right itself when laid on its side. All animals were euthenized at stage 1.

### Statistical Analysis of Clinical Outcomes

Clinical outcomes were studied on the same set of data originating from five concurrent treatment groups allowed to survive to terminal illness. These outcomes included disease onset and life span as well as disease progression based on consecutive motor scores (motor score curves) and Kaplan-Meier survival. In one type of analysis, these outcomes were compared among the four groups of subjects treated with live cells but with variable immunosuppression, i.e. FK506 (n=42), FK506+rapamycin (n=11), FK506+rapamycin+MMF (n=5), and anti-CD4 antibodies (n=5). Variance in disease onset and life span was analyzed with one-way ANOVA, followed by a Fisher LSD post hoc test. Variance in motor score curves was analyzed by repeated-measures ANOVA, followed by Fisher LSD testing. Statistical analysis of Kaplan-Meier survival was based on Logrank testing.

In a second type of analysis, clinical outcomes were compared between two groups of animals, both of which were immunosuppressed with FK506+rapamycin, but one had received live cells whereas the other was treated with dead cells. Disease onset and life span between live- and dead-cell groups were compared with a Student's t-test. Disease progression was compared in the two groups by analyzing differences in motor score and Kaplan-Meier survival curves as described above.

### Histology, Immunocytochemistry (ICC), and Microscopy

At end-stage disease and at one week and one month post-grafting, mice were euthenized with an overdose of sodium pentobarbital (5 mg/100 g i.p.) followed by intracardiac perfusion with 4% freshly depolymerized, neutral-buffered paraformaldehyde. Spinal cord tissue blocks containing the entire lumbar region were dissected and immersion-fixed in paraformaldehyde for an additional 4 hr at RT after removing the dura. Tissues were then equilibrated in 30% sucrose and sectioned at the transverse plane (30 μm) on a freezing microtome.

ICC studies focused on the differentiation of human NSCs, the structural integration of NSCs, and the characterization of type and intensity of immune response to the graft. Many of these experiments required dual-label immunofluorescence. After permeabilization with 0.1% Triton X-100 and non-specific site blocking with 5% normal serum from the same species as the secondary antibodies, sections were incubated in primary antibodies in 1 mg/ml BSA with 0.1% Triton X-100 (4° C., overnight). Primary antibodies were used to address human (graft) versus mouse (host) cell identity, neuronal, astrocytic, and oligodendrocytic phenotype specification, and the type and intensity of host-versus-graft cellular response (see Supplemental Table 1). The presence of human cells in mouse tissues can be reliably traced with antibodies against human nuclear antigen (HNu) [64]. Control sections were stained by replacing the primary antibodies with pre-immune IgG from the same species of origin.

Antigen-antibody binding sites were revealed with Cy2- or Cy3-conjugated secondary goat or donkey IgGs directed against the species of origin of the corresponding primary antibodies (1:200; Jackson ImmunoResearch Laboratories, Inc., West Grove, Pa.). In most cases, Cy3-conjugated goat or donkey anti-mouse IgG was used to trace the human cell marker HNu and Cy2-conjugated goat or donkey IgG was used for various cellular markers. Secondary antibody incubations were performed for 2-4 hours at RT. All sections were counterstained with the DNA dye DAPI (blue) and then dehydrated and coverslipped with DPX. In the few cases that our experimental design required triple labeling, the third secondary antibody was coupled with the blue fluorescence dye 7-amino-4-methyl-coumarin-3-acetic acid (AMCA, 1:200; Jackson ImmunoResearch Laboratories, Inc., West Grove, Pa.) and DAPI counterstain was omitted. Sections were studied with a Zeiss Axiophot microscope equipped for epifluorescence, and images were captured with a Spot RT Slider digital camera (Diagnostic Instruments, Inc., Sterling Heights, Mich.). Confocal microscopic images were captured and optically resectioned in the x and y axes using a Zeiss LSM 410 unit.

### EXAMPLES

### Example 1

### In Vitro Differentiation and Trophic/Tropic Profile of Human NSCs

NSCs prepared for grafting were propagated as a monolayer culture in the presence of bFGF and delivered to animals within 24 hours post-bFGF withdrawal. At that time, all cells expressed the NSC marker nestin (FIG. 1A), approximately 5% were immunoreactive for the neuronal precursor-specific marker PSA-NCAM, and less than 1% expressed the neuronal markers. TUJ1 and MAP2 or the astroglial marker GFAP. With continued culture in the absence of bFGF, about 50% of cells acquired neuronal phenotypes as shown by MAP-2 immunoreactivity and cytological profile (FIG. 1B), and some differentiated into GFAP (+) astrocytic profiles (FIG. 1C) within two weeks. Very few cells expressed oligodendrocyte/Schwann cell markers within this time frame.

The expression of representative neurotrophic factors and neuregulins was studied by real-time PCR at 0, 14, 29, and 42 days post-bFGF withdrawal, i.e. during the differentiation phase (FIG. 1D). Among neurotrophic factors, GDNF and BDNF transcripts increased 3- and 7-fold respectively, whereas VEGF increased 15-fold by day 29; significant sustained increases were also noted for IGF-1 (FIG. 1D). Among neuregulins (NRG), NRG1 and NRG3 transcripts, particularly the latter, were also found to have significantly increased. The expression of all these factors declined after 4 weeks, a pattern that may reflect some cellular deterioration that is common in long-term culturing of mature neurons.

### Example 2

### Survival and Migration of Human NSCs in Rat Spinal Cord

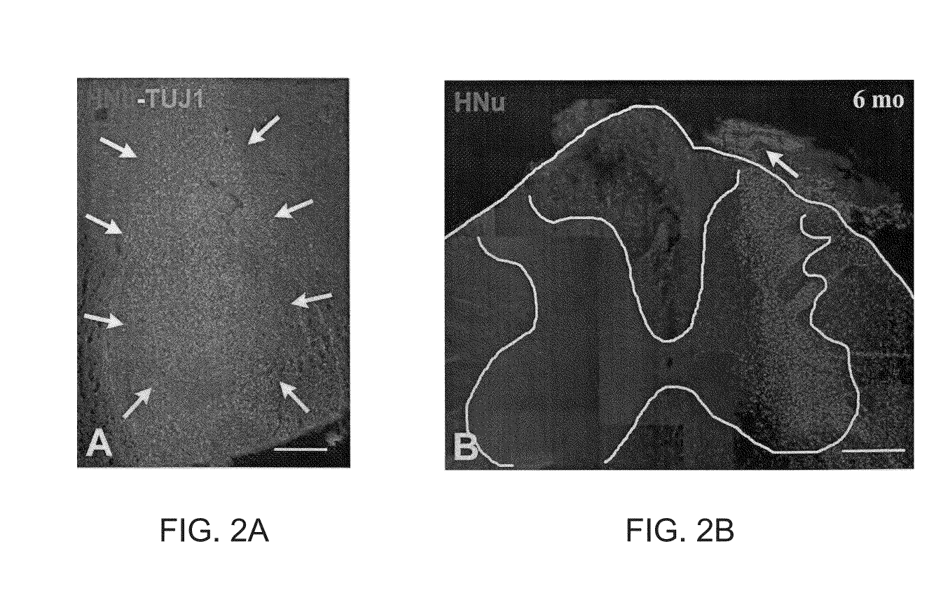

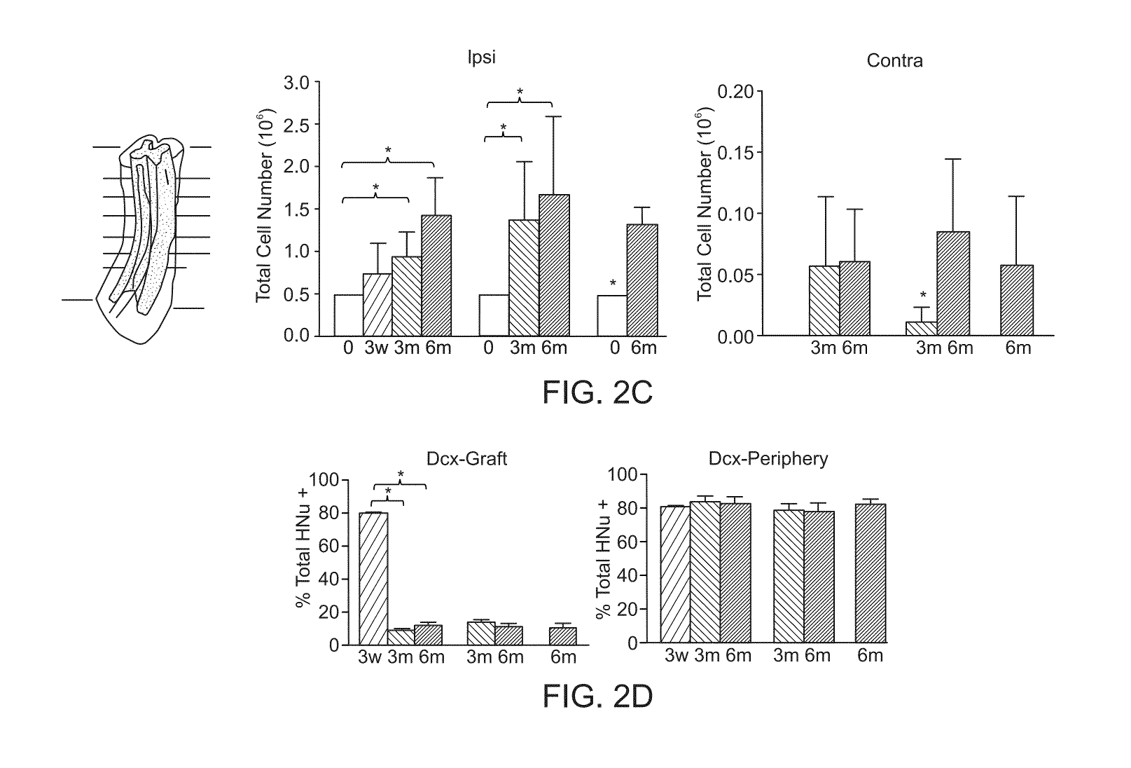

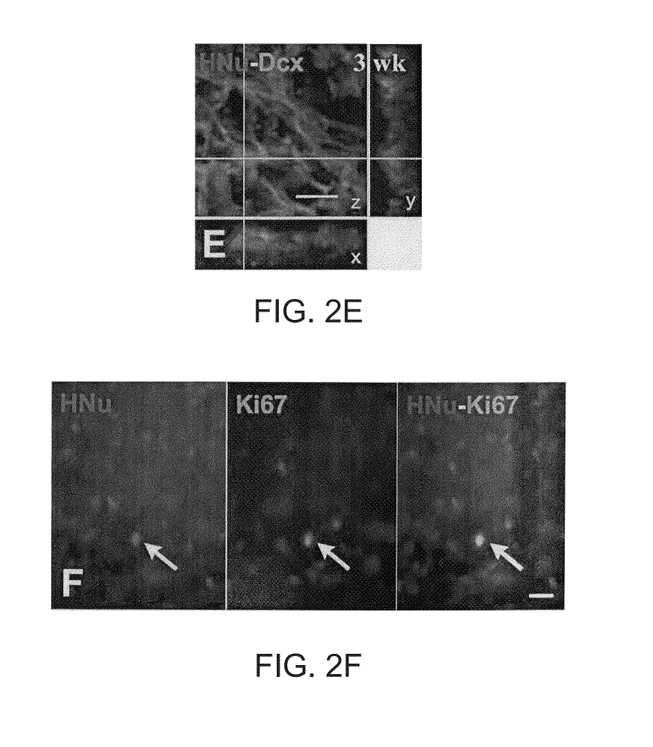

The examination of all stained sections from lesioned and control animals shows a robust engraftment and excellent long-term survival of NSCs in the adult spinal cord environment (FIG. 2A-B). Stereological estimates of surviving HNu (+) cells show an average of 1.5×10⁶ cells present in the L4-L5 cord 6 months post-grafting. This figure represents a 3-4 fold increase of the cell population present in the initial graft (FIG. 2C) and implies that the initial graft survived well and underwent, on average, two mitotic divisions. This estimate is consistent with a low frequency (3-5%) of HNu (+) cells that also expressed the nuclear antigen Ki67, a marker of all phases of cell cycle minus GO, in all experimental conditions studied in this paper (FIG. 2F). Ki67-positive cells were found randomly dispersed across the graft area without evidence of clustering in specific sites.

Comparison among animals surviving for 3 weeks, 3 months and 6 months shows a significant tendency of 566RSC cells to migrate away from the initial grafting sites and populate both gray and white matter, as well as the proximal end of ventral and dorsal roots in the avulsion cases. Approximately 3% of NSC-derived cells were found in the contralateral side, mostly in the white matter. There were more cells in the contralateral side in avulsion-injured than in HCA-injured animals 3 months post-grafting (FIG. 2C, right-hand panel). The migratory disposition of 566RSC cells is further confirmed with the expression of doublecortin (Dcx), a microtubule-associated protein that serves as a specific marker for migrating neuronal precursors [22], by about 80% of grafted cells three weeks post-grafting (FIG. 2D). Dcx expression is reduced to 10-15% of HNu (+) cells at the grafting sites at 3 and 6 months but remains very high (˜80%) in HNu (+) cells peripheral to the grafts up to 6 months post-grafting, evidence for continuous migration (FIG. 2E).

In summary, human NSCs survive very well in the spinal cords of nude rats with minimal further mitotic activity irrespective of the presence or absence of lesion, and migrate extensively into the ipsilateral and contralateral spinal cord.

### Example 3

### Differentiation of Human NSCs into Neuronal and Non-Neuronal Cells: Parenchyma Versus Meninges

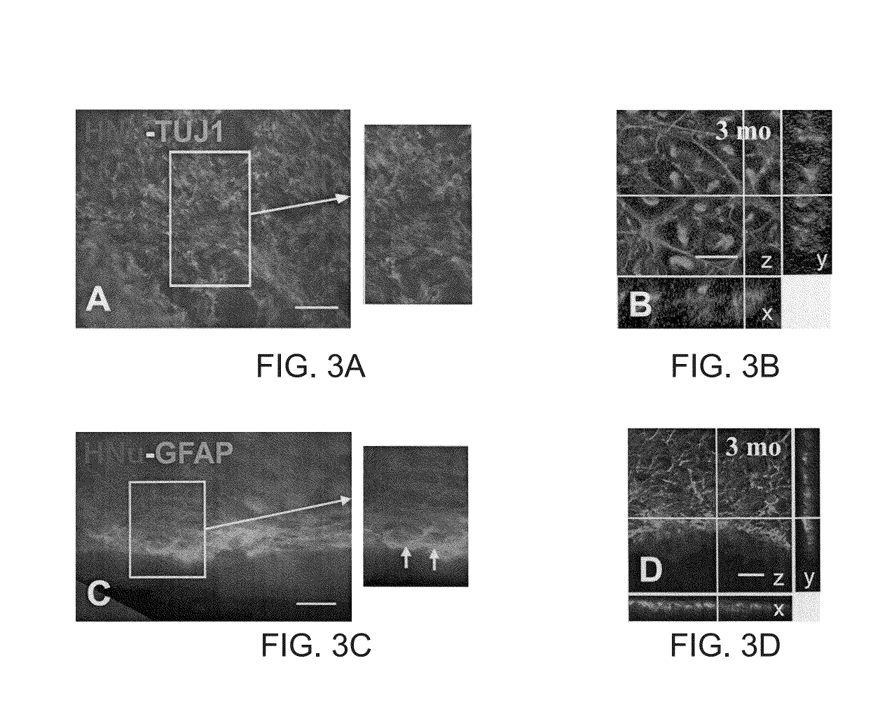

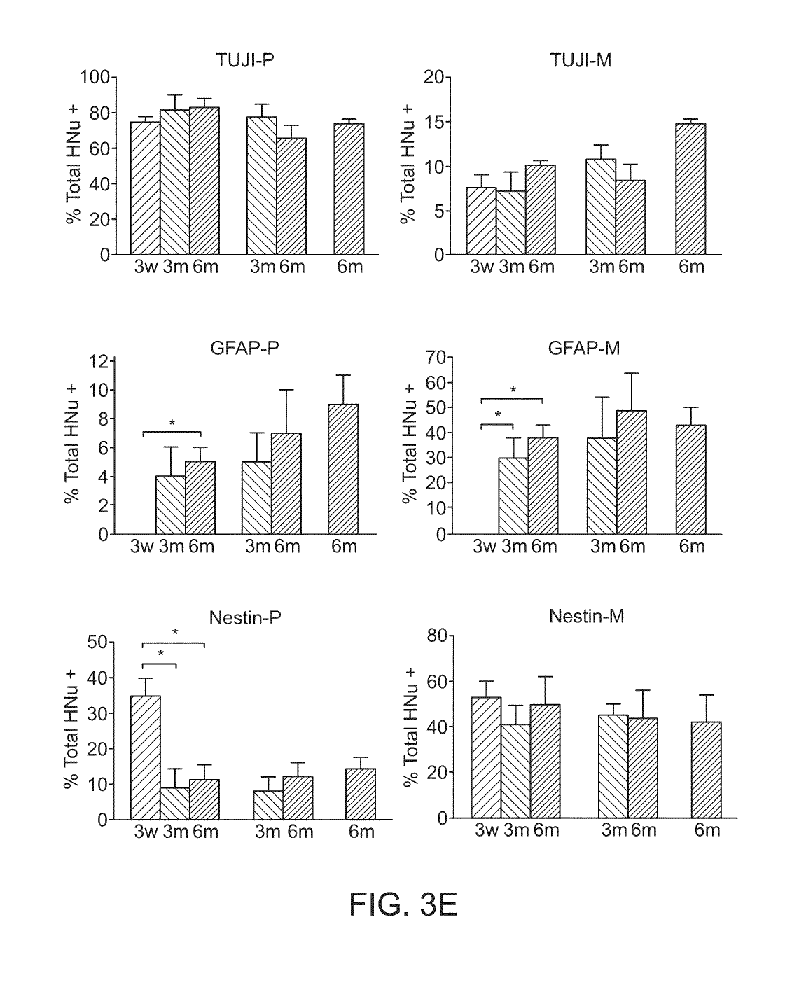

Within the L4-L5 parenchyma, the vast majority of NSCs promptly entered a neuronal lineage, as evidenced by very high rates of TUJ-1 immunoreactivity at 3 weeks (75%) and at 3 months (78-80% in various treatment groups) and 6 months (67-82%) post-grafting (FIG. 3A-B; E). These TUJ-1 (+) cells have round or bipolar cell bodies and an average diameter of 10 μm (FIG. 3A-B). Rates of TUJ-1 differentiation did not differ significantly among treatment groups and were consistent with results from dual ICC for HNu and the neuronal nuclear epitope NeuN. In addition, there were no significant differences in rates of TUJ-1 (+) NSC-derived cells among various time points, evidence that the establishment of a neuronal lineage occurred very early in the life of these grafts. Compared to neuronal markers, the appearance of astrocytic phenotypes was slower. HNu (+) cells do not show significant GFAP immunoreactivity at 3 weeks post-grafting. By 3-6 months, ˜5% of grafted HNu (+) cells in the avulsion cases and a slightly higher proportion of cells in HCA− and sham animals stained for GFAP (FIG. 3E, left-hand panel). The astrocytic cytology of these GFAP (+) cells is confirmed by confocal microscopy (FIG. 3D). A declining percentage of NSCs showed nestin immunoreactivity throughout the time course studied here. There was a significant reduction in numbers of nestin (+) cells between 3 weeks and 3 months post-grafting, but ˜11-14% of HNu (+) cells remained nestin (+) even at 6 months, a pattern indicating that a small portion of NSCs persisted at a precursor state long after grafting (FIG. 3E, left-hand panel).

In a number of animals, the meninges adjacent to the ventral horn were included in the grafts. At those sites, patterns of differentiation were different from the parenchymal sites. For example, the rate of GFAP (+), astrocyte-like, NSC-derived cells was higher (30-50%) compared to parenchymal grafts, and there were more cells persisting in a nestin (+) state (40-53%) (FIG. 3C-D; E, right-hand panel). The rate of neuronal differentiation was very low at those sites; 6-11% of HNu (+) cells colocalized TUJ1 immunoreactivity (FIG. 3E, right-hand panel) and this was independently confirmed with dual staining for HNu and NeuN. In sections that were dually labeled for human nestin and GFAP, we established that the two phenotypes were expressed by distinct populations of NSC-derived cells, i.e. the vast majority of GFAP (+) cells were nestin (−) and nestin (+) cells were GFAP (−). This pattern suggests that, in our preparations, nestin expression was not an essential feature of astrocytes, and that nestin (+) cells persisted, to a substantial degree, as neural stem or precursor cells. In both parenchymal and meningeal locations we were able to find HNu (+) cells that also expressed A2B5, a ganglioside antigen present in the common glial precursor O-2A occasionally, at 3 months as well as 6 months post-grafting. However, immunoreactivity for the early oligodendrocyte markers CNPase and O4 and the mature oligodendrocyte markers gal C and Rip was negative in all cases.

As a proof of the concept that the predominantly neuronal differentiation of NSCs is not dependent on the athymic state of nude rats, we performed a small study in which we grafted the lumbar cord of normal rats. These animals had to be treated with FK506 to prevent xenograft rejection. Two, months post-grafting, animals were prepared exactly as the nude rats and the neuronal differentiation of 566RSC cells was explored with dual immunofluorescence using NeuN and TUJ1 as neuronal markers. As in the case of nude rats, the vast majority of NSCs had differentiated into neuronal cells (FIG. 4A-4B).

In concert, the fate of grafted NSCs depends on location, i.e. the parenchymal environment promotes a neuronal differentiation, whereas the meningeal environment appears to facilitate the progression towards astrocytic differentiation or allows NSCs to remain in an immature, nestin (+) state. Irrespective of location, differentiation of human NSCs in the oligodendrocytic lineage is negligible.

### Example 4

### GABAergic and Cholinergic Neurotransmitter Phenotypes Expressed by Grafted NSCs

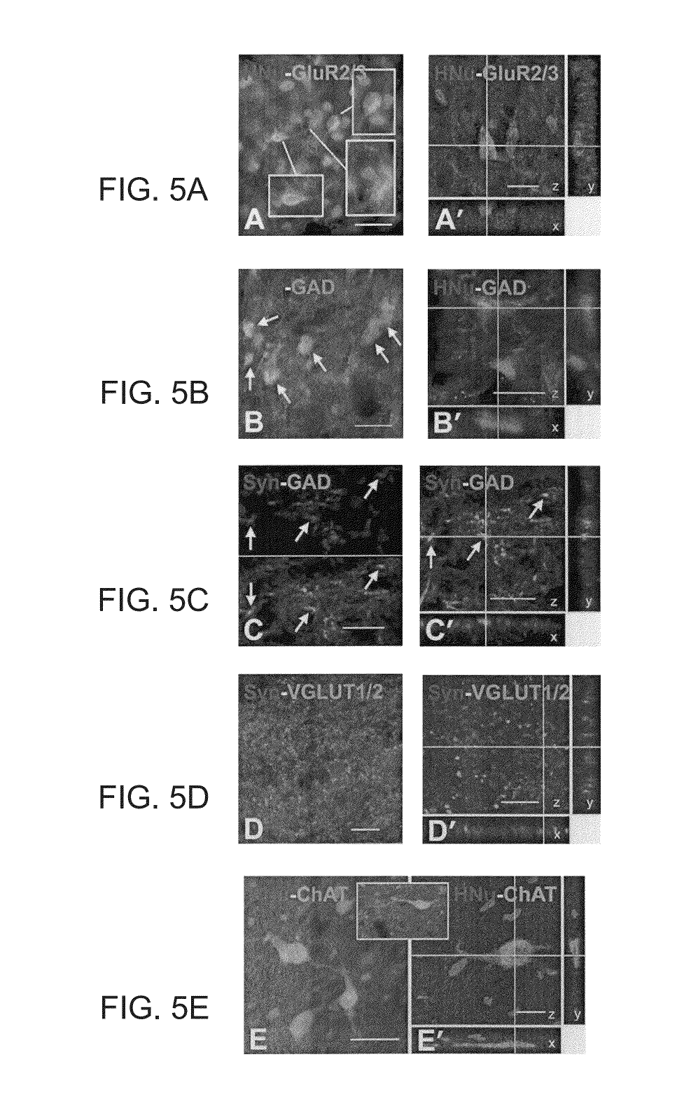

Because of the predominant neuronal fate of grafted NSCs excitatory, inhibitory (GABA) and cholinergic neurotransmitter markers were examined in order to ascertain the degree of differentiation of cells into the neuronal lineage (FIG. 5A-5E). Almost without exception, grafting sites were markedly enriched in metabotropic glutamatergic (FIG. 5A), and GABAergic (FIG. 5B) neurotransmitter markers. Individual bipolar NSC-derived cells were seen to express strong immunoreactivity for GluR2/3 and GAD and to be contacted by terminals enriched in these two neurotransmitter markers, indication that graft-derived cells engage in both excitatory and inhibitory neurotransmission. Both GluR2/3 and GAD immunoreactivity became first apparent in grafting sites at 3 months and persisted without appreciable changes at 6 months post-grafting.

Examination of sections stained for HNu, GAD, and GluR2/3 showed a large number of NSC-derived neurons colocalizing both GAD and GluR2/3. To distinguish between GABAergic and glutamatergic phenotypes in the nerve terminals, we performed dual ICC for human-specific synaptophysin (to mark graft-derived terminals) and GAD or vesicular glutamate transporter type 1 and 2 (VGLUT-1/2). GAD and VGLUT-1/2 are sensitive and selective markers for GABAergic and glutamatergic neurons respectively [23,24], and terminal staining is especially robust in the spinal cord (FIG. 5C-D). To label a maximal number of glutamatergic terminals, antibodies against VGLUT-1 and VGLUT-2 were used in combination. Large numbers of GAD and VGLUT-1/2 (+) terminals were seen at the grafting sites, but fields of VGLUT-1/2 (+) puncta only partially overlapped the graft (FIG. 5D). In multiple fields in sections dually stained for human-specific synaptophysin and GAD, we found that a majority of synaptophysin (+) terminals colocalized GAD immunoreactivity by confocal microscopy (FIG. 5C′). In contrast, no colocalization of human synaptophysin with VGLUT-1/2 immunoreactivity was observed in the same terminals, despite dense apposition of individually labeled terminals (FIG. 5D′).

Using GAD immunoreactivity as a reliable marker of GABAergic neurons, we counted HNu and GAD (+) profiles and calculated rates of dually labeled cells in the total population of HNu (+) cells at 6 months, i.e. the longest survival time examined. At that time point, a significant percentage of HNu (+) cells in all three experiment groups (avulsion: 60.5+/−0.47; HCA lesion: 56.4+/−3.19; sham: 49.57+/−4.04) were also GAD immunoreactive. Frequency of differentiation did not vary significantly by type of treatment.

A very small percentage of HNu (+) cells (less than 1%), first appearing at 3 months and consistently seen at 6 months post-grafting, colocalized ChAT immunoreactivity. ChAT (+) neurons were larger than other neuronal HNu (+) cells (15-25 μm in diameter) and displayed multipolar cytologies (FIG. 5E).

These findings indicate that a majority of NSC-derived neurons develop and sustain stable bipolar cytologies and GABAergic phenotypes for at least 6 months after grafting. These cells are contacted by GABAergic terminals from other graft and host neurons and glutamatergic terminals from the host. A small, but consistent percentage of graft-derived neurons evolve into larger multipolar neurons with cholinergic phenotypes.

### Example 5

### Developmental Neuronal Phenotypes Expressed by Grafted NSCs

At 3 and 6 months post-grafting, we examined our graft preparations for the expression of genes that are specific for developing motor neurons in ventral horn, such as the LIM homeodomain transcription factors Islet-1 and HB9 [25,26]. In addition, we screened our preparations for the expression, by graft-derived cells, of the common neurotrophin receptor p75^{NTR}, a robust marker of motor neurons during the period of axonal extension/developmental cell death [27,28].

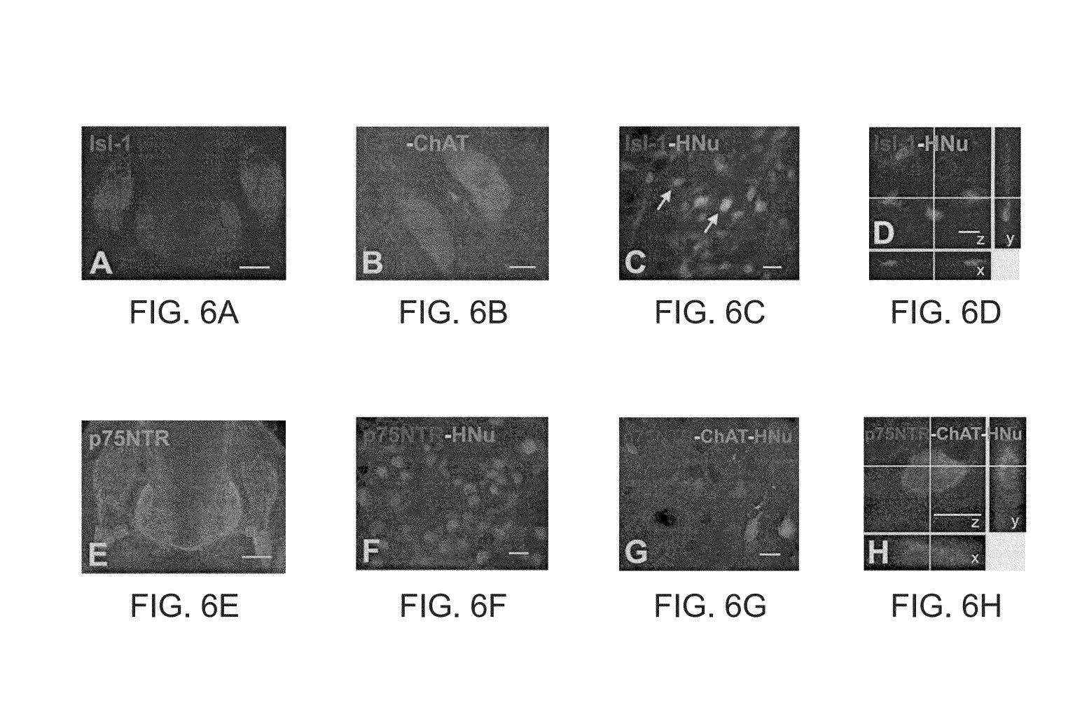

Islet-1, a marker for motor neurons throughout their life span, was robustly expressed in rat spinal cord at E13 (FIG. 6A) and, less intensely, in host α-motor neurons (FIG. 6B). Islet-1 immunoreactivity was also present in a large number of HNu (+) cells (FIG. 6C-D), including cells labeled for both HNu and ChAT. In our hands, HB9 ICC resulted in inconsistent staining of HNu (+) cells, although it did detect nuclear and some cytoplasmic immunoreactivity of E11-E12 motor neurons in the medial column. A majority of small bipolar (putative GABAergic) HNu (+) neurons was found to colocalize p75^{NTR} immunoreactivity (FIG. 6F). However, only few multipolar cholinergic neurons originating in the graft expressed p75^{NTR} immunoreactivity (FIG. 6G-H).

In concert, a majority of human NSC-derived neurons express the common neurotrophin receptor p75^{NTR} and many also express homeodomain transcription factors characteristic of developing motor neurons. Expression of p75^{NTR} is not selectively associated with either GABAergic or cholinergic neurotransmitter phenotypes expressed by differentiated NSCs, although it may be more frequent in non-cholinergic neurons.

### Example 6

### Structural Integration of Human NSCs in Rat Spinal Cord: Synapses, Axons, and Long Projections into Ventral Roots

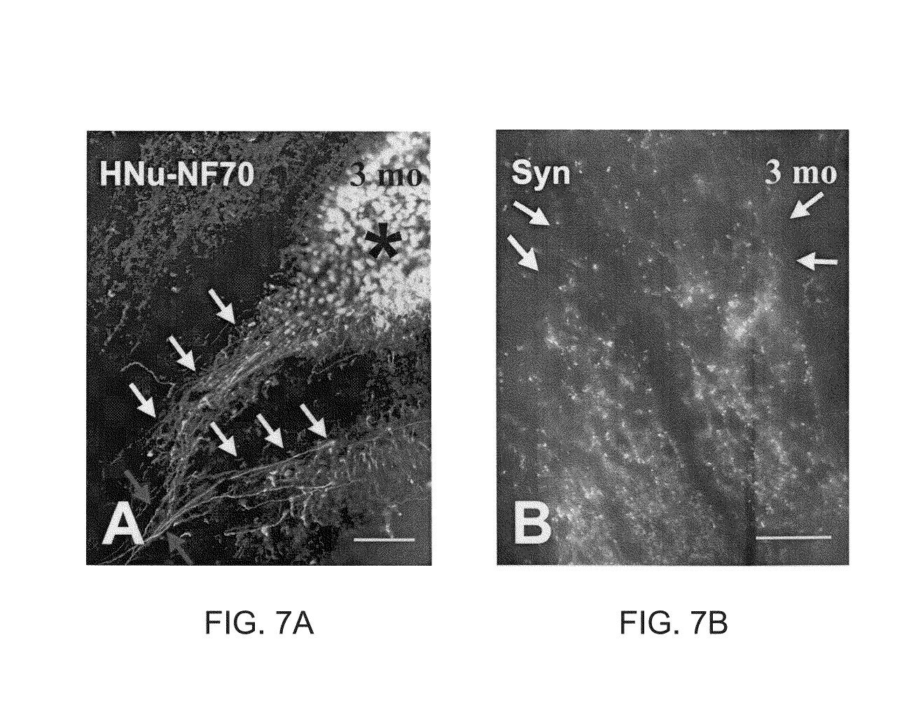

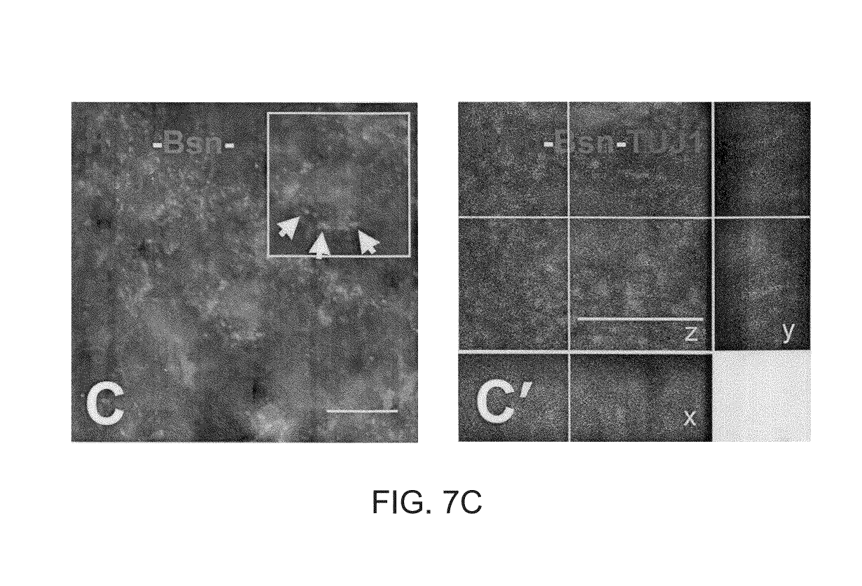

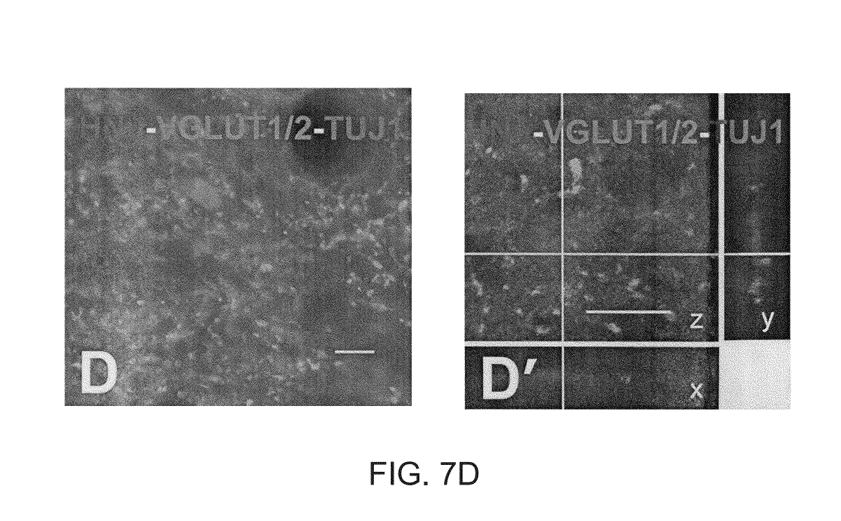

By 3 months post-grafting, HNu (+) neurons elaborate prolific axons (FIG. 7A) and synapses (FIG. 7B) that can be specifically linked to graft origin with antibodies selective for human synaptophysin and neurofilament (NF) proteins. Axons bundle in groups and form secondary tracts aiming at the ventral root exit zone (FIG. 7A). However, the ability of HNu (+) neurons to elaborate highly differentiated structures like axons and synapses does not necessarily imply their capacity to integrate within the host circuitry. Therefore, to differentiate between host and graft inputs, we used a monoclonal antibody for the presynaptic protein-Bassoon (BSN) that selectively recognizes rat and mouse, but not human, epitopes. In sections stained for HNu (to establish graft origin), TUJ-1 (to establish neuronal differentiation), and Bassoon (to detect terminals from host rat axons), we found that most HNu (+), TUJ-1 (+) cells in parenchymal locations were contacted by large numbers of synaptic boutons of rat origin (FIG. 7C-C′), i.e. evidence that the host species (rat) massively innervates graft-derived (human) neuronal cells. Because graft-derived terminals are negative for markers of glutamatergic neurotransmission, i.e. VLGUT1/2 immunoreactivity, VLGUT1/2 ICC is an independent probe useful in differentiating between graft and host terminals. By combining, in triple ICC protocols, staining for HNu and TUJ-1 (together marking graft-derived neurons) with mixed staining for VLGUT1 and VGLUT2 (together marking most host-derived glutamatergic terminals), we found dense appositions between graft-derived neurons and host glutamatergic terminals (FIG. 7D-D′). In preparations stained for VLGUT1/2, GFAP and HNu, we never observed such contacts between host terminals and graft-derived astrocytic profiles.

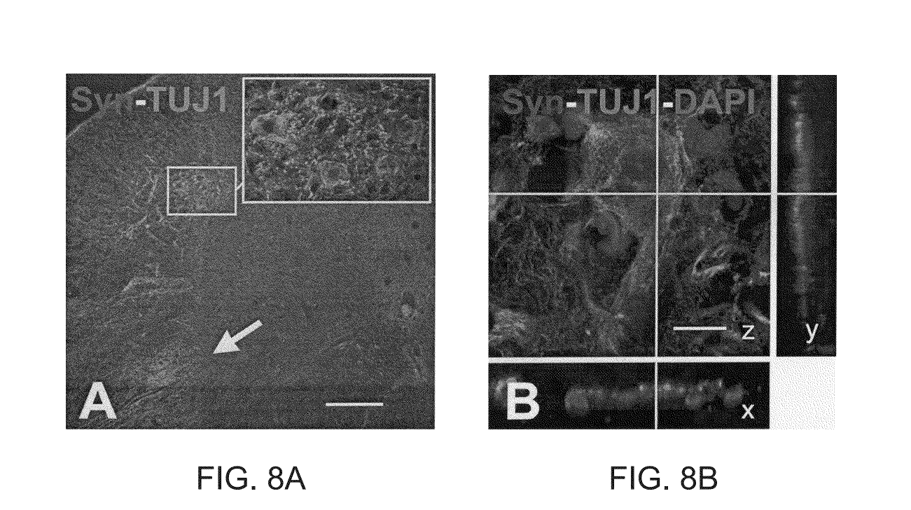

Conversely, preparations stained with human-specific synaptophysin and TUJ-1 or ChAT revealed dense terminal fields comprised of multiple small boutons that were apposed to host neurons, including large and small motor neurons both on the side of grafting as well as the contralateral side. Large numbers of host motor neurons were seen to be contacted in such a fashion by graft-specific terminals (FIG. 8A-B). Treatment variance (avulsion, HCA lesion, or sham treatment) did not seem to affect the presence or absence of such dense reciprocal contacts. In sections stained for human synaptophysin and GFAP, we did not observe any appositions between graft-derived terminal specializations and astrocytes. This evidence suggests the elaboration of reciprocal innervation between graft-derived and host neurons.

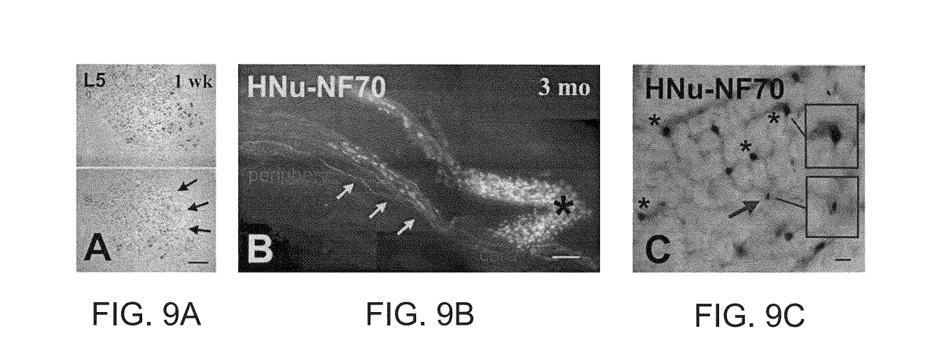

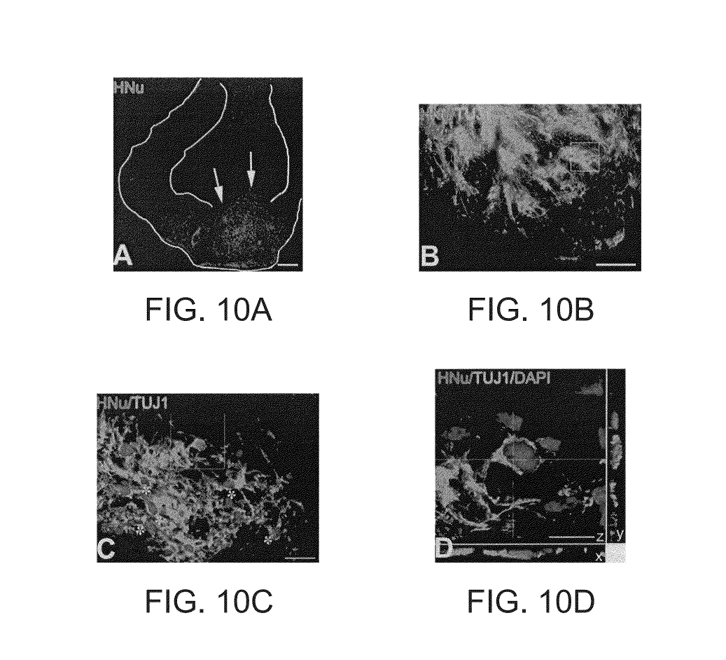

For NSCs differentiating in the spinal cord and elaborating axons, a critical question is whether axons derived from these cells can traverse the ventral root and, possibly, the peripheral nerve, and eventually re-establish some form of connection with muscle targets. This is a complex problem with many facets that fall beyond the scope of this paper, yet some observations made on our HCA and sham treatment in which the ventral roots were preserved (FIG. 9A) are relevant to the issue of graft differentiation and integration. First, in most L4 and L5 roots we examined 3-6 months post-lesion, we found human NF70 (+) axons coursing along the root. Because of the advantages related to penetration of reagents, teased root preparations universally revealed many more axons than whole root mounts (FIG. 9B). There were no apparent differences in density of these newly extended axons between HCA− and sham animals. Second, in most cases we also found HNu (+) cells that had migrated along with the new axons (FIG. 9B). Many of these migrating HNu (+) cells were also TUJ-1 (+). Cross-sections of ventral roots confirmed the presence of both migrating NSCs and graft-derived axons in motor roots (FIG. 9C).

In summary, NSC-derived neurons not only develop differentiated neurotransmitter phenotypes in the adult spinal cord, but also elaborate axons and synaptic specializations, and they engage in dense reciprocal innervations with host spinal cord neurons. In addition, ventral roots in animals with intact parenchyma-root continuity contain graft-derived axons and migrating HNu (+) cells.

### Example 7

### Survival, Neuronal Differentiation and Structural Integration of Human NSCs in the Spinal Cord of SOD1 G93A Rats

Human NSCs were identified by their HNu protein signature and their phenotypic fates were tracked with dual ICC for HNu and epitopes specific for neural precursor, neuronal, and glial cells. At the end of experiments in SOD1 G93A rats, human NSCs showed robust engraftinent and excellent long-term survival (FIG. 10A). A majority of HNu (+) cells (70.4%) had differentiated into the neuronal lineage, based on their colocalization of TUJI (FIG. 10B-D). Approximately one-fifth (19.2%) of HNu (+) cells colocalized nestin, and very few (1.3%) HNu (+) cells were positive for GFAP.

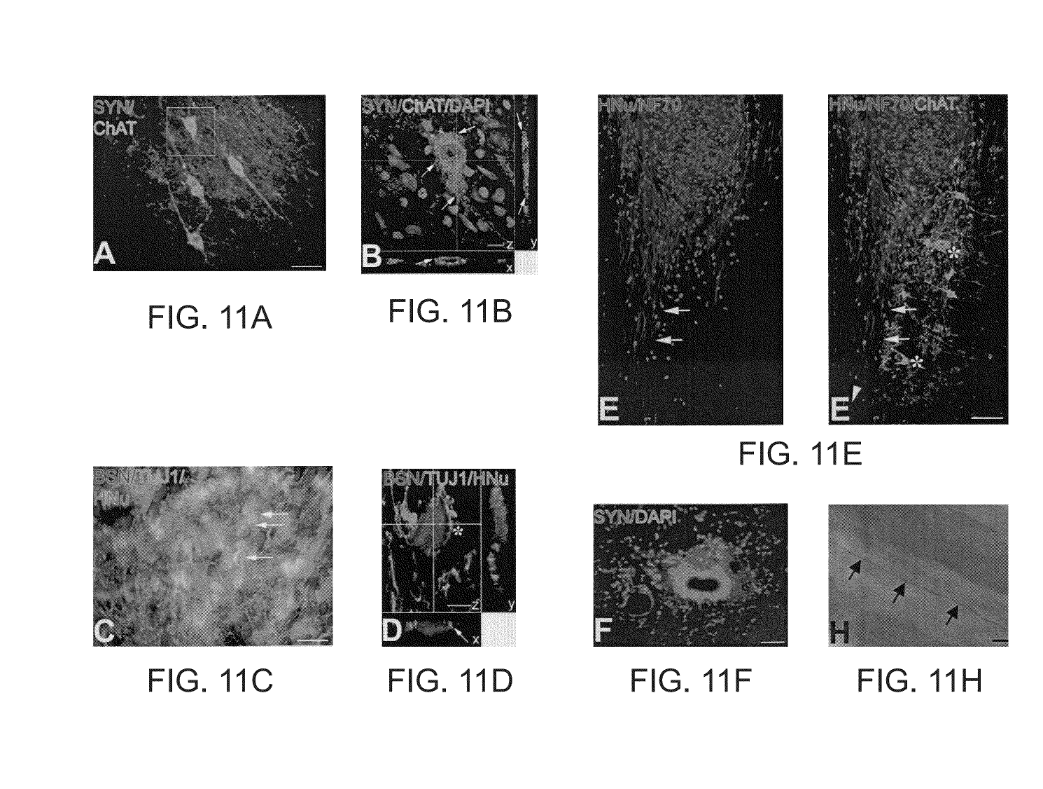

The capacity of human NSCs to integrate within the host circuitry was tested with perikaryal markers for graft/host cells and markers selective for either host or graft axons/terminals. A large number of ChAT (+) host motor neurons was found to be contacted by human synaptophysin (+) synaptic boutons of graft origin (FIG. 11A-B). Conversely, a large number of rat-derived synaptic boutons was seen in close proximity to graft-derived neurons in preparations stained with either a mixture of antibodies for VGLUT1/2 (present in host, but not in graft terminals), HNu and TUJ1 or a combination of antibodies for the presynaptic protein Bassoon (that recognizes rat and mouse but not human epitopes), HNu, and TUJ1. Synaptic contacts of rat nerve terminals on graft-derived neurons were not as common as human nerve terminals on host motor neurons (FIG. 11C-D).

Besides projecting locally to innervate host motor neurons, human NF70 (+) axons from differentiated NSCs formed bundles that coursed in the ventral funiculus and crossed over at least 1-2 spinal segments. The preference of these axonal bundles for white versus gray matter was evident in preparations stained for markers that revealed the boundaries of ventral horn (FIG. 11E). A dense plexus of human NF70 (+) axons and human synaptophysin (+) terminals was consistently observed in juxtaposition to ependymal cells (FIG. 11F), likely resulting from targeted migration of differentiated NSCs rather than the formation of de novo graft-to-ependyma projection. Only a few human NF70 (+) axons were found in teased or en block preparations of ventral roots (FIG. 11G).

### Example 8

### NSC Grafts into the Lumbar Cord of SOD1 G93A Rats Prolong Life Span and Delay Motor Neuron Death and Disease Onset and Progression

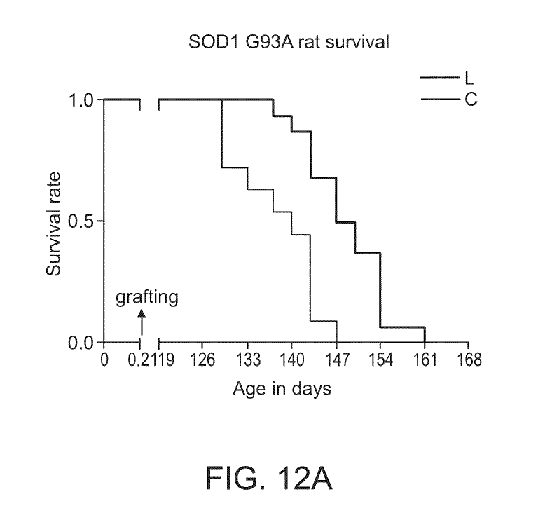

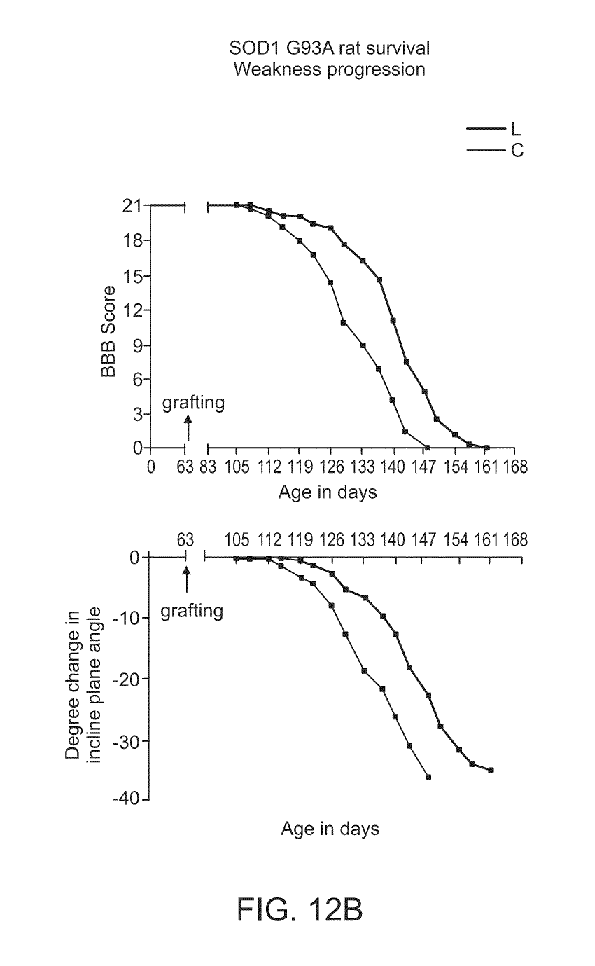

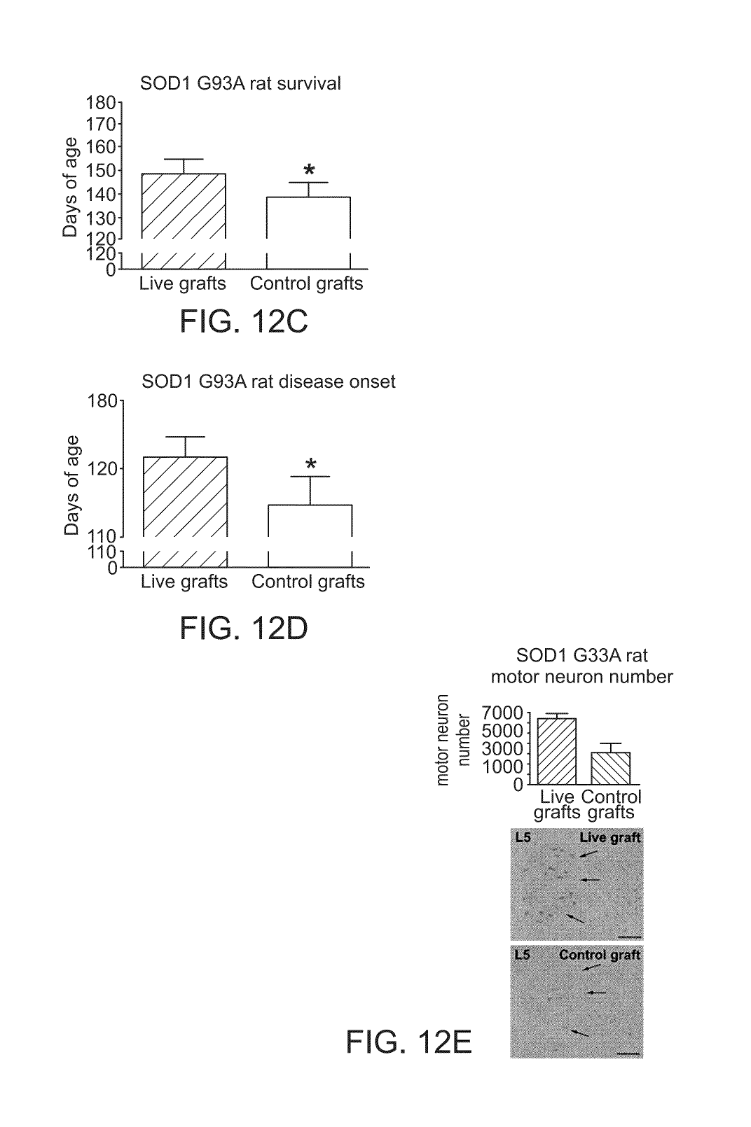

Animals grafted with live NSCs showed increased survival by both Kaplan-Meier (FIG. 12A) and end-point analysis; the average life span for animals grafted with dead NSCs was 138 days, whereas rats grafted with live NSCs lived for 149 days (FIG. 12C). Average time-to-disease-onset was 115 days for animals that received dead cells and 122 days for animals that were grafted with live NSCs (FIG. 12D). Time plots of BBB open field and inclined plane test scores show a significantly slower progression in muscle weakness in animals grafted with live cells compared to animals that received dead NSCs (FIG. 12B).

The effect of NSCs on motor neuron survival in the lumbar protuberance (L3-S1) of Tg rats was examined in a small group of animals that received live or dead NSCs and were sacrificed at 128 days of age. Stereologically estimated numbers of α-motor neurons were 6,418 for animals that received live NSCs and 3,206 for rats grafted with dead NSCs, i.e. there were twice as many neurons in the lumbar protuberance of experimental compared to control animals of the same age (FIG. 12E).

### Example 9

### Molecular Correlates of Clinical and Biological Effects of Human NSCs in SOD1 G93A Rats: Evidence for the Expression and Release of Neurotrophins and Trophic Cytokines, with Emphasis on GDNF

To explore potential mechanisms of neuroprotection afforded by human NSCs on degenerating motor neurons, we studied the expression and release of BDNF and GDNF, two peptides with classical trophic effects on mammalian motor neurons [53-56], by Western blotting and ELISA using both spinal cord preparations and CSF samples.

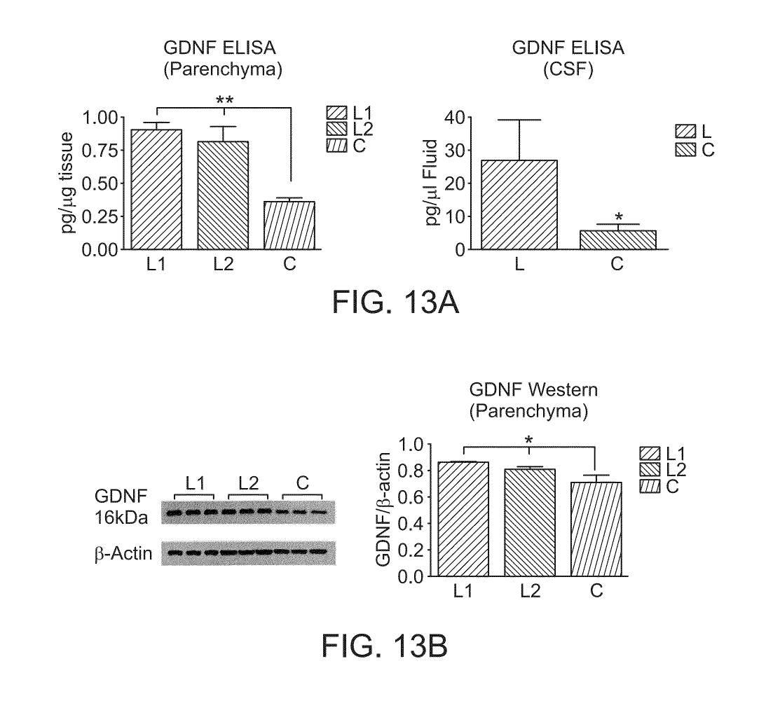

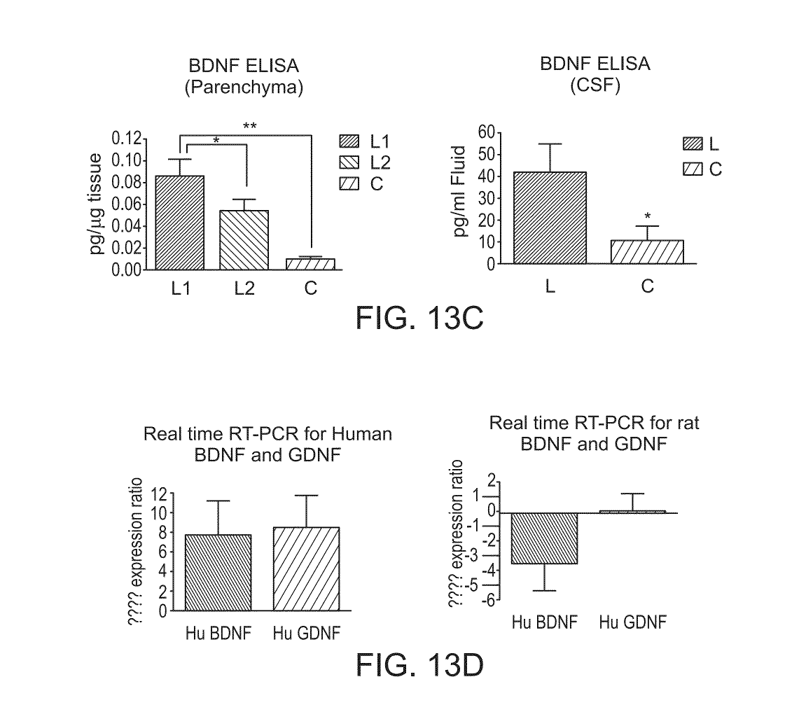

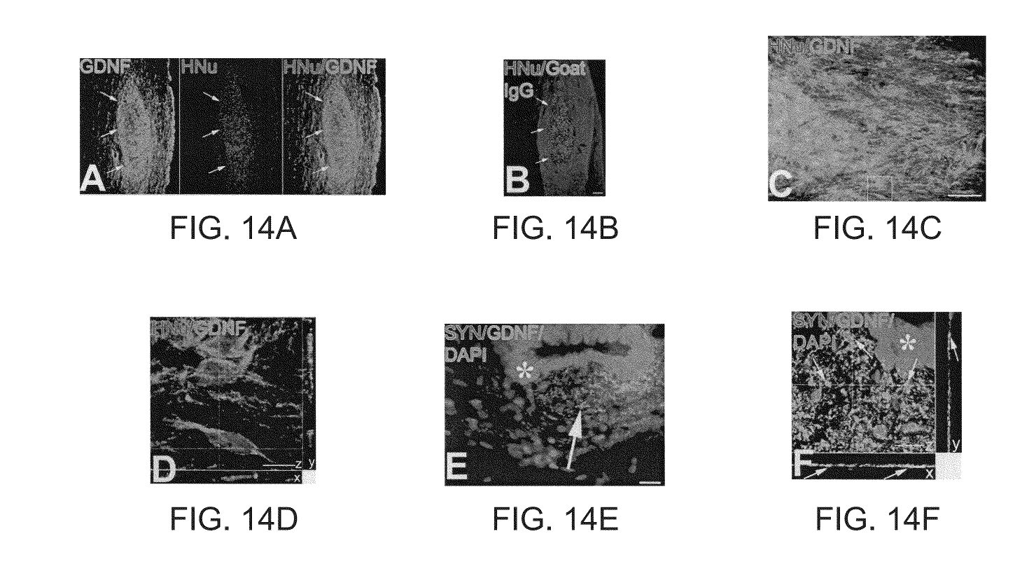

In the case of GDNF, ELISA shows a three-fold increase in the release of this trophic peptide in the spinal cord (FIG. 13A, left) and a five-fold increase in GDNF secreted in the CSF (FIG. 13A, right) in animals with live NSCs. Western blotting confirms the ELISA pattern of increase and shows a normalized GDNF density of 0.860 in live-cell grafts and 0.708 in dead-cell grafts (FIG. 13B). In the case of BDNF, there was an eight-fold increase in BDNF concentration in the spinal cord (FIG. 4C, left) and a four-fold increase in the CSF (FIG. 13C, right) of animals grafted with live cells. BDNF was not detectable by Western blotting in our hands.

The ELISA data suggest a more widespread secretion of GDNF compared to BDNF in animals grafted with live NSCs, especially in the CSF. To explore the source of the excess trophic peptide, i.e. differentiate between graft (human) and host (rat) origin, we performed real-time RT-PCR analysis for GDNF and BDNF mRNA expression on samples from live- and dead-cell graft sites using human- and- rat-specific primers (FIG. 13D). In addition, we studied the expression of VEGF, an angiogenic trophic peptide with evident trophic effects in mammalian motor neurons [57]. Normalized human GDNF and BDNF mRNA expression in spinal cords grafted with live cells was several-fold higher than that in NSCs prior to grafting; we found an 8-fold change for human BDNF and a 9-fold increase for human GDNF. Rat BDNF mRNA expression in spinal tissues with live NSC grafts decreased by 3.5-fold, whereas rat GDNF expression remained essentially unchanged when compared to tissues with dead NSC grafts. Human VEGF mRNA expression was found to be ˜2 times higher in grafted tissues compared to a pre-grafting sample of NSCs, and rat VEGF mRNA expression decreased ˜3 times compared to levels in spinal cords with dead-cell grafts.

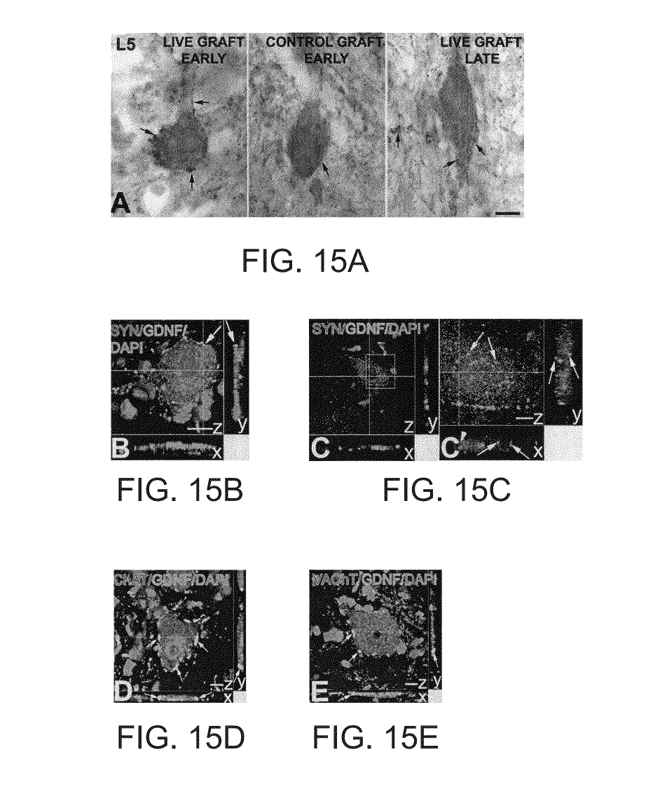

The graft origin of GDNF, i.e. the trophic peptide with the greatest upregulation and widespread secretion in subjects with live grafts, was further supported with GDNF ICC. In animals with live grafts, the vast majority of grafted HNu (+) cells were found to express immunoreactive GDNF (FIG. 14A-D). GDNF immunoreactivity was shown to be localized within NSC-derived terminals, e.g. in the dense terminal fields around ependymal cells (FIG. 14E). Moreover, spinal cords with live grafts exhibited a high density of GDNF-immunoreactive boutons attached to cell bodies and proximal dendrites of host motor neurons. These boutons were especially prominent in animals with early motor neuron disease (BBB score 19 or higher) and were less prominent in animals with advanced disease (BBB score 6 or lower) (FIG. 15A). In contrast to terminals near ependymal cells, the vast majority of NSC terminals contacting host motor neurons did not contain immunoreactive GDNF (FIG. 15B-C). On the other hand, GDNF (+) terminals on motor neuron cell bodies were found to colocalize ChAT and VAChT, i.e. classical markers of cholinergic nerve terminals, in early disease stages (FIG. 15D-E). The size, shape, and neurotransmitter identity of these GDNF (+) terminals identifies them as cholinergic C-boutons, i.e. terminals of segmental spinal origin that participate in local motor circuits [58]. In concert, although most graft-derived differentiated cells express GDNF and the excess GDNF expression in grafted cords is of human origin, the terminals of graft-derived cells on host motor neurons rarely contain GDNF immunoreactivity. The increased GDNF immunoreactivity within synapses belonging to host neurons in an environment with substantially increased human and unchanged rat GDNF expression raises the suspicion of a transsynaptic transfer of GDNF from graft cell terminals onto host motor neurons and/or interneurons [59].

### Example 10

### Combined Immunosuppressive Agents or CD4 Antibodies Ameliorate Cell-Mediated Rejection and Improve Human NSC Graft Survival in SOD1-G93A Mice

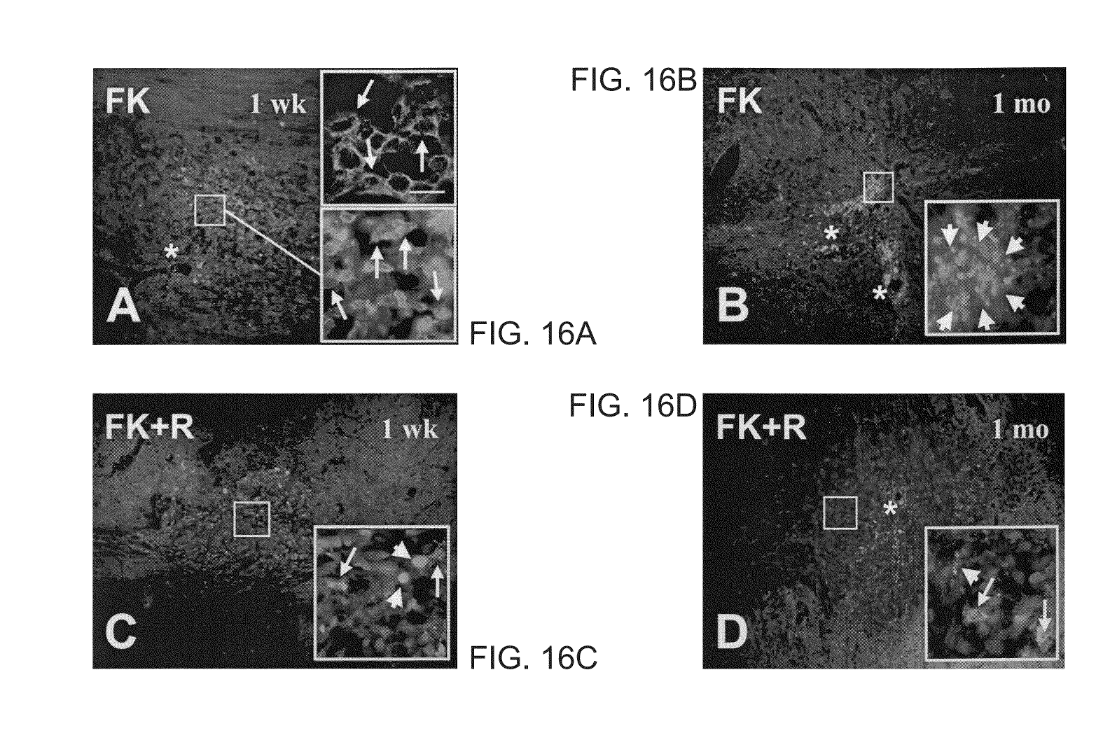

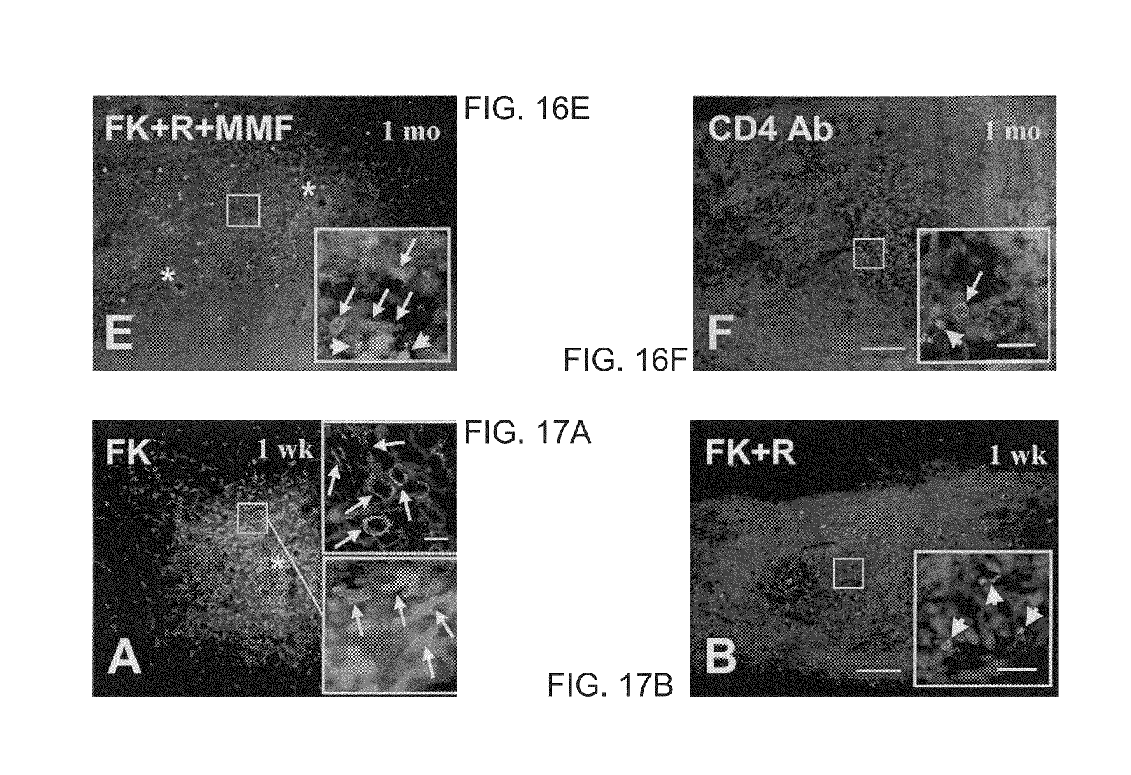

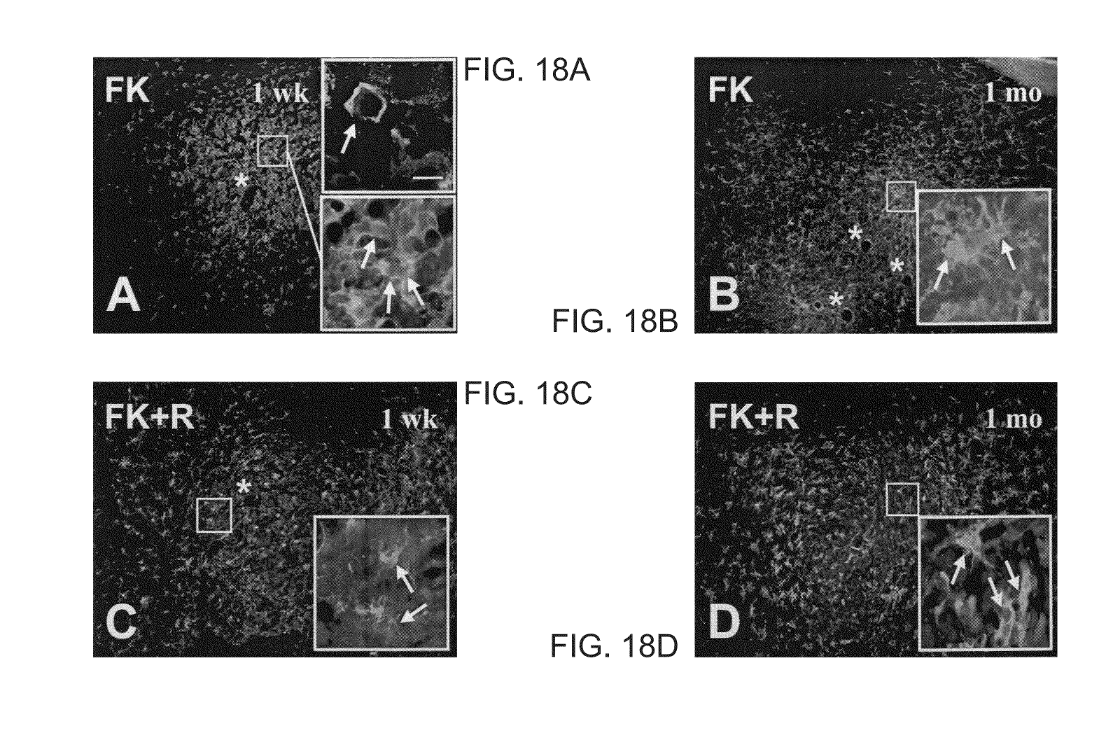

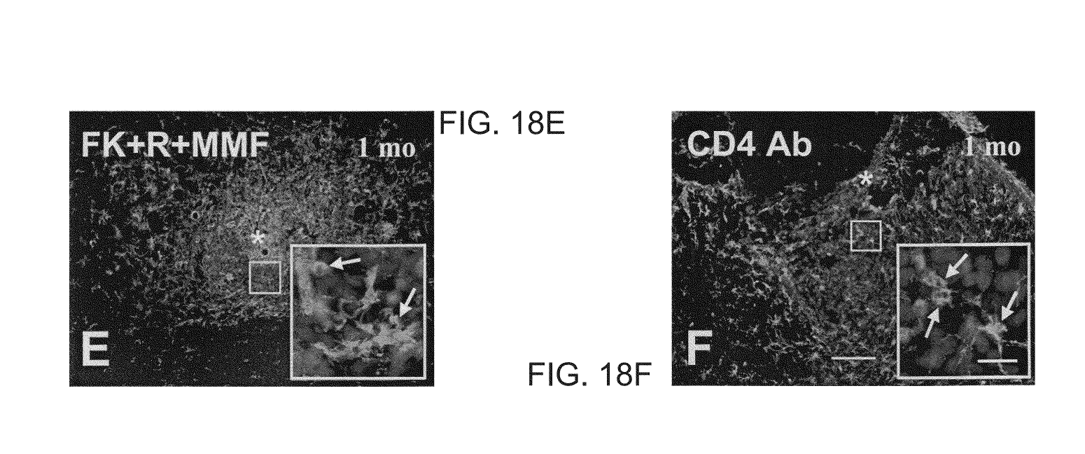

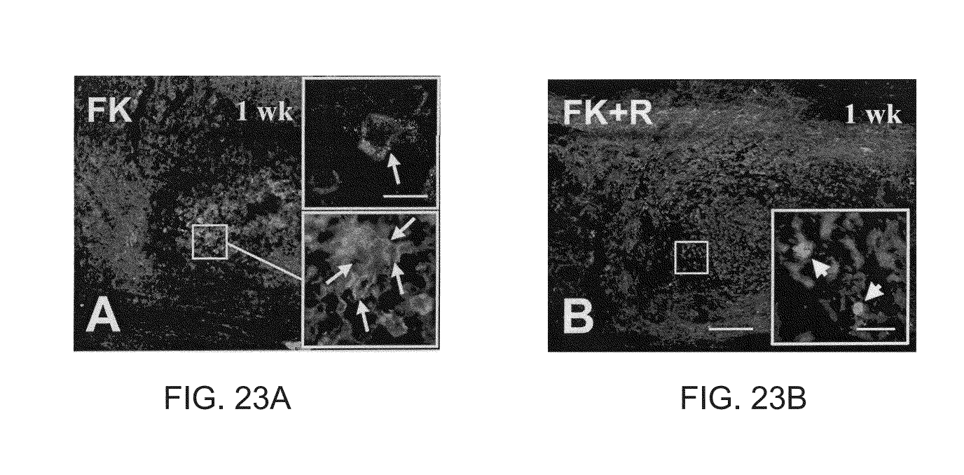

Graft rejection was studied with ICC for HNu (to mark graft-derived cells) and protein epitopes marking blood-borne immune cells i.e. lymphocytes and natural killer cells or resident microglia/macrophages. Blood-borne cells were detected with antibodies against CD4 and CD8 surface antigens of T-cells (FIGS. 16A-16F and FIGS. 17A-17B) and an antibody against a surface epitope present in NK cells (DX5, FIGS. 23A-23B) [65]. Microglial cells were labeled with an antibody against the Iba-1 epitope of a microglia-specific calcium-binding protein expressed by these cells in all functional states including resting, activated, and phagocytic microglia [66,67] (FIGS. 18A-18F). Cytological features and anatomical relationships with blood vessels also helped in the identification of immune cells. For example, blood-borne cells cluster primarily around blood vessels, although this pattern is less distinct with advanced rejection. Under all circumstances, blood-borne cells are rare in host tissues surrounding the grafts.

CD4 (+) and CD8 (+) cell recruitment to grafting sites were similar in terms of cytology and perivascular clustering with all treatments and at all time points, but the CD4 response was predominant (FIGS. 16A-16F and FIGS. 17A-17B). NK cells were not seen frequently in our preparations. A moderately intense NK cell response was seen in subjects treated with FK506 alone, primarily in perivascular locations with little parenchymal invasion (FIGS. 23A-23B). The cytology of these DX-5 (+) NK cells resembled that of CD4 (+) and CD8 (+) cells. In animals treated with combined immunosuppressive regimens, DX-5 immunoreactivity appeared primarily as debris without obvious cellular localization (FIG. 23A, inset). CNS microglia was typically seen to invade the graft from surrounding tissues replete with microglial cells at various stages of transformation (FIGS. 18A-18F). As we established [68], reactive microglia is featured by a scant cytoplasm and long, highly ramified processes, whereas phagocytic glia has retracted, thick processes and a more substantial cytoplasm volume.

With FK506 treatment alone, HNu staining fails to reveal graft-derived nuclei as early as 1 week post-grafting (FIGS. 16A, 17A, 18A and FIGS. 23A-23B). Grafting sites are featured by a diffuse, non-nuclear HNu immunoreactivity often seen in close proximity to CD8 (+) lymphocytes (FIG. 1A, insets) and, to a lesser degree, to NK cells (FIG. 23, insets), but not CD4 (+) cells (FIG. 17A, insets). HNu immunoreactivity is often localized within the cytoplasm of microglial phagocytes (FIG. 18A, insets). One month post-grafting, CD8 (FIG. 16B) and CD4 (not shown) immunoreactivities remain strong in the graft region, but they are localized within small round structures without cellular organization that probably represent cellular debris. Very few live CD4 or CD8 (+) cells are visible at the graft sites (FIG. 16B), a pattern indicating that active T-cell rejection is over by that time. Iba-1(+) microglial cells with phagocytic cytology are numerous at the graft site 1 week post-grafting (FIG. 18A). At one month, microglial reaction remains strong, but many of these Iba-1 (+) cells exhibit cytologies consistent with activated microglia (FIG. 18B).

Combined treatment with FK506 and rapamycin has an impressive effect on graft survival, based on the presence of numerous dense, nuclear HNu (+) profiles in all our experimental subjects (FIGS. 16C-D, 17B, FIGS. 23A-23B, and FIG. 18C-D). CD4 and CD8 (+) cell infiltration is markedly diminished; many such cells appear to have died based on the early presence of CD4 or CD8 (+) debris. Microglial response is moderate; the overwhelming majority of Iba-1 (+) cells have the appearance of activated, but not phagocytic, microglia (FIGS. 18A-18F; compare insets between panels C and A). A very low level of CD4 or CD8 (+) response is present at 1 month post-grafting (FIG. 16D). Microglial reaction is still strong, and there is an apparent increase in the presence of macrophages at 1 month (FIG. 18D) and well into end-stage disease (not shown). The addition of MMF to the regimen of FK506 plus rapamycin had no apparent effects in graft survival and infiltration by CD4/CD 8 (+) cells (FIG. 16E) or by microglia/macrophages (FIG. 18E) compared to the double immunosuppressive regimen.

The use of CD4 antibodies also significantly promoted the viability of HNu (+) cells. Invasion of the graft by blood-borne lymphocytes (FIG. 16F) or microglial cells (FIG. 18F) was significantly attenuated. As with combined immunosuppressive treatments, the cytology of most Iba-1 (+) cells resembled that of activated, but not phagocytic, microglia (FIG. 18F, inset).

In concert, the combination of FK506 and rapamycin with or without MMF or the use of CD4 antibodies, suppressed CD4- and CD8-cell infiltration and also significantly attenuated and delayed microglia-mediated phagocytosis.

### Example 11

### Combined Immunosuppressive (Wilts and CD4 Antibodies Improve Disease Outcomes in SOD1-G93A Mice Wafted with Human NSCs

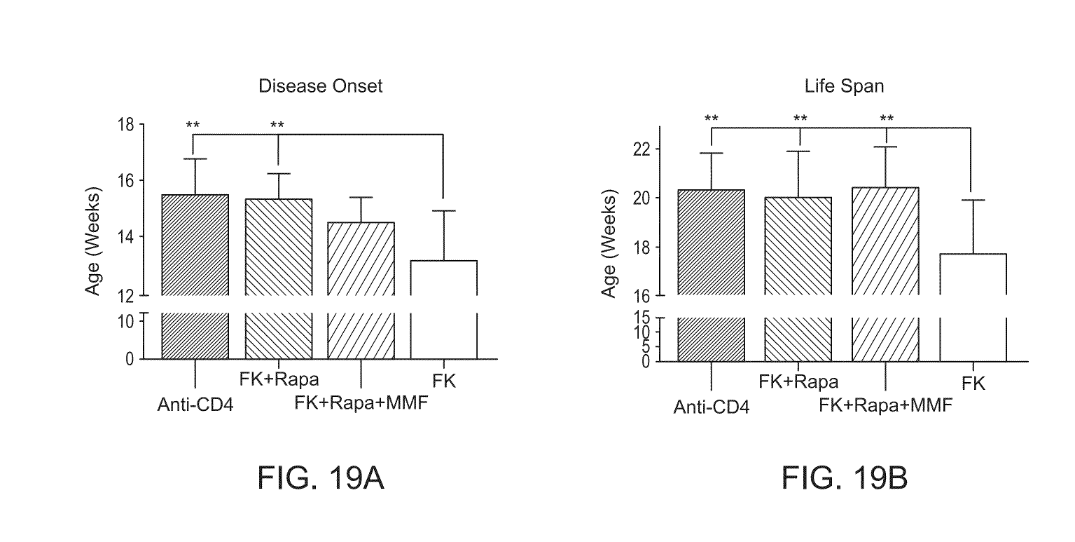

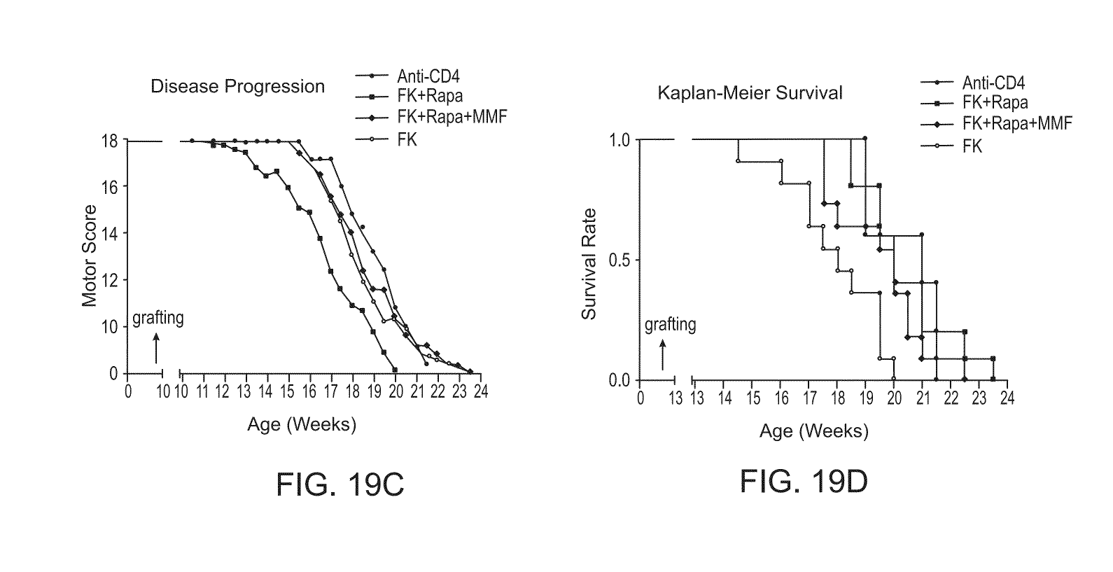

When treated with combined immunosuppressive drugs or anti-CD4 antibodies, SOD1-G93A mice grafted with live NSCs showed delayed disease onset, improved motor scores, and longer life spans compared to FK506 monotherapy (FIGS. 19A-19B). Mice treated with FK506 alone had an average disease onset of 13.2±1.8 weeks. Treatments with the GK1.5 antibody, FK506+rapamycin, and less so with FK506+rapamycin+MMF all delayed time to disease onset (15.5±1.3 weeks, 15.3±1.0 weeks, and 14.5±0.9 weeks, respectively) compared to the FK506 group (FIG. 19A). Post-hoc testing reveals that individual comparisons between the anti-CD4 or FK506+rapamycin groups and FK506 group are significant, but the difference between FK506+rapamycin+MMF and FK506 groups is not. Treatment with anti-CD4 antibody, FK506+rapamycin, and FK506+rapamycin+MMF significantly extended the life span of NSC-grafted animals compared to FK506 treatment alone (20.3±1.5 weeks, 19.7±1.9 weeks, and 20.4±1.7 weeks as compared with 17.6±2.0 weeks) (FIG. 19B). Motor score testing also showed a delayed loss of muscle strength in the anti-CD4, FK506+rapamycin, and FK506+rapamycin MMF groups compared to FK506 treatment group (FIG. 19C). Logrank testing of Kaplan-Meier survival curves also revealed significant differences among anti-CD4, FK506+rapamycin, FK506+rapamycin+MMF, and FK506 groups. The use of Logrank testing within pairs of treatment groups revealed significant differences between FK506 monotherapy and any of the other treatment groups (FIG. 19D). Based on the above data, the combined FK506+rapamycin regimen appears to optimize prevention of graft rejection, at least for the purpose of generating clinically meaningful differences in the time framework of motor neuron disease in SOD1-G93A mice.

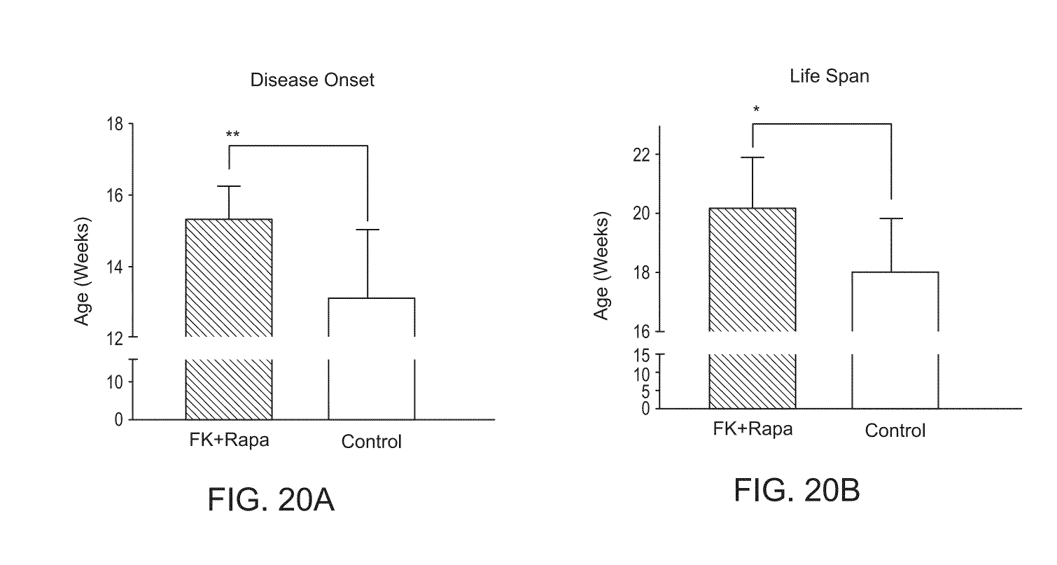

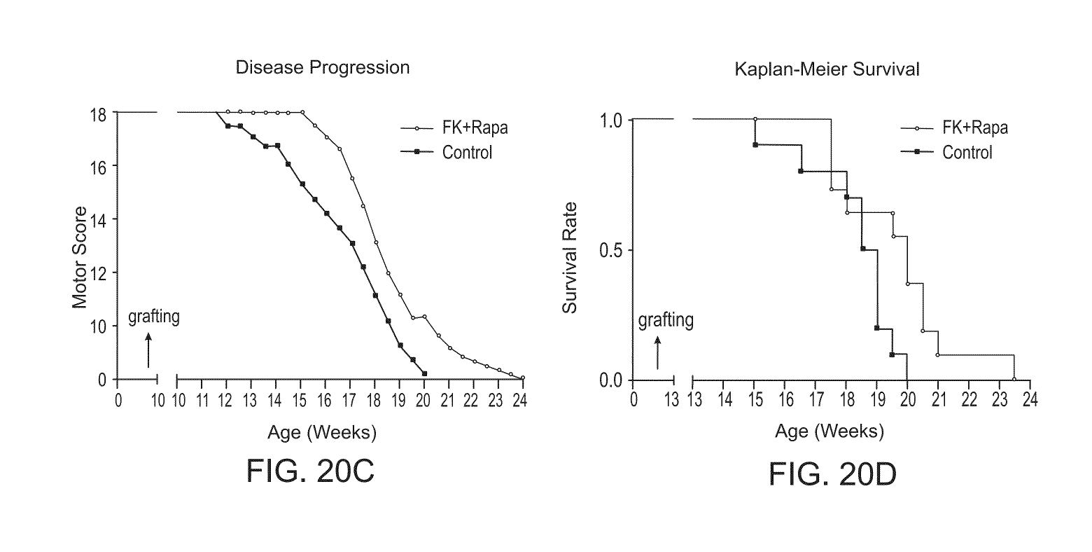

To control for the potential effects of immunosuppressants on clinical parameters of motor neuron disease, we compared disease onset, motor score, and life span data between animals grafted with live versus dead human NSCs, all of which were treated with FK506+rapamycin. In animals grafted with live cells, disease onset was delayed by 2.1 weeks (FIG. 20A), and life span was extended for 1.7 weeks (FIG. 20B) compared to animals that had received dead cells (disease onset: 15.3±1 versus 13.1±1.9 weeks; life span: 19.7±1.9 versus 18.0±1.8 weeks). Motor score testing shows a later onset and slower progression of weakness in animals treated with live NSCs (p=0.02) (FIG. 20C) and Kaplan-Meier survival analysis shows a significant difference between live- and dead-cell groups (p=0.001) (FIG. 20D). These differences indicate that immunosuppressants influence key clinical parameters of motor neuron disease in SOD1-G93A mice via their protection of NSC survival and not via a direct protective effect on host motor neurons.

### Example 12

### Grafted Human NSCs Survive to End-Stake Disease, Differentiate Predominantly into Neurons, and Establish Synaptic Contacts with Host Neurons

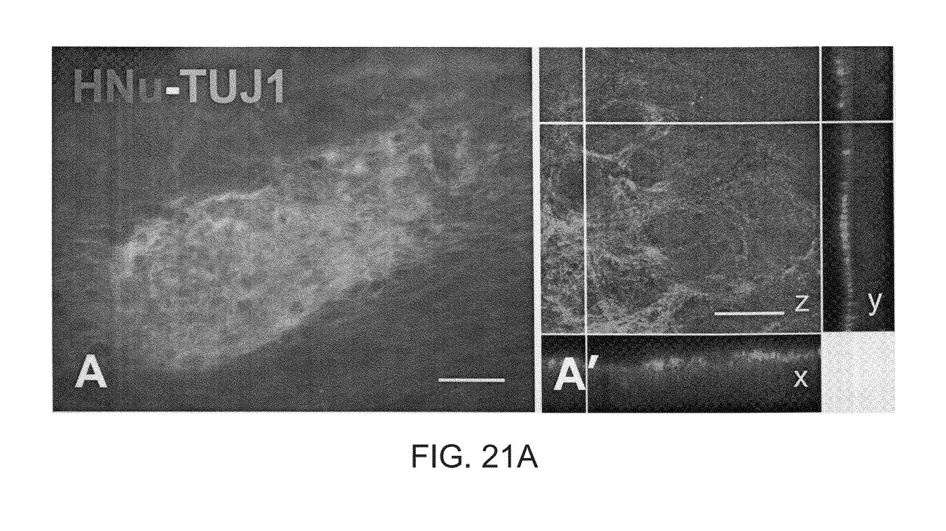

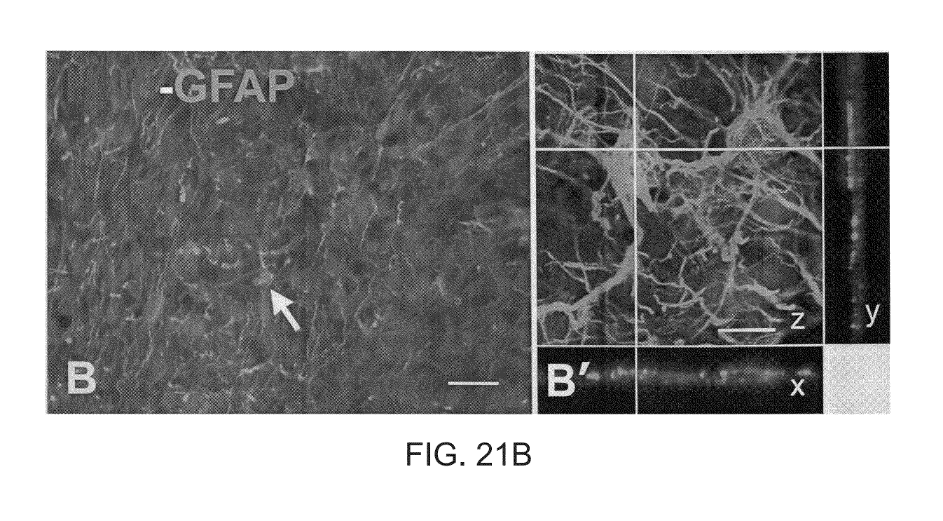

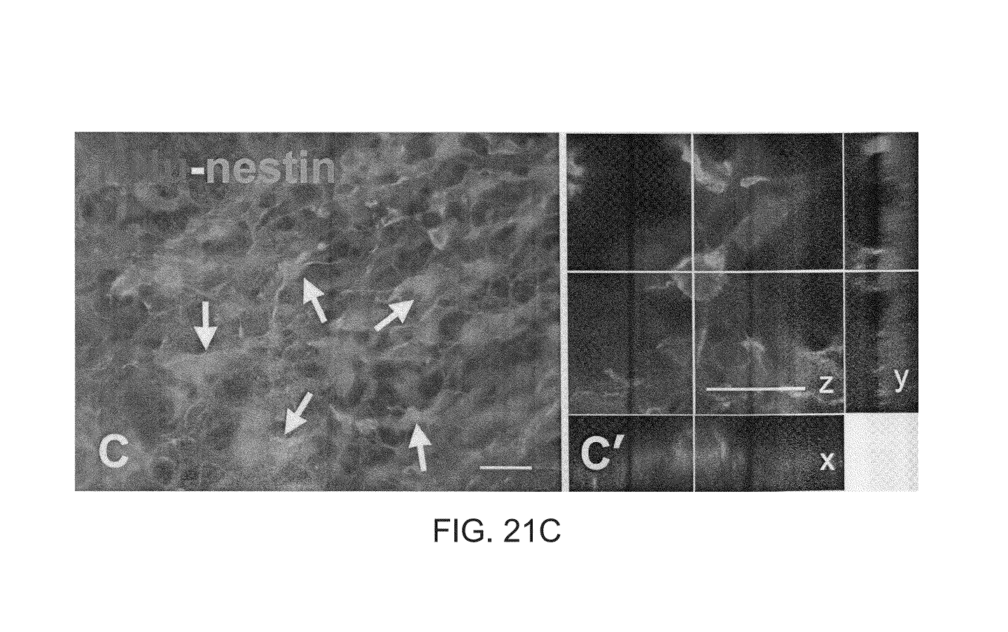

In end-stage animals (more than two months post-grafting) treated with combinations of immunosuppressive agents or CD4 antibodies, the vast majority of human NSCs had differentiated into TUJ1 (+) neurons (FIG. 21A). Confocal microscopy confirmed the colocalization of HNu (red) with TUJ1 (green) in the same cells (FIG. 21A′). Although numerous GFAP (+) astrocytic processes were present in the graft site (FIG. 21B), the colocalization of GFAP immunoreactivity with HNu (+) nuclei within the same cells was a rare phenomenon in the majority of grafts located within the spinal cord parenchyma (FIG. 21B). This profile suggests that most GFAP (+) processes at the graft site are of host origin. However, in portions of grafts located very close to meninges (pia), the frequency of HNu (+) cells colocalizing GFAP increased, including the occasional clustering of double-labeled cells (FIG. 21B′); this pattern shows a degree of plasticity in NSC differentiation determined by local factors in the spinal cord microenvironment. HNu (+) nuclear profiles did not colocalize with O4 or Rip immunoreactivity within the same cells, a pattern suggesting that human NSCs did not differentiate in the oligodendrocyte or Schwann cell lineage in our experimental settings (data not shown). A small number of human NSCs remained undifferentiated as nestin (+) cells (FIG. 21C, C′).

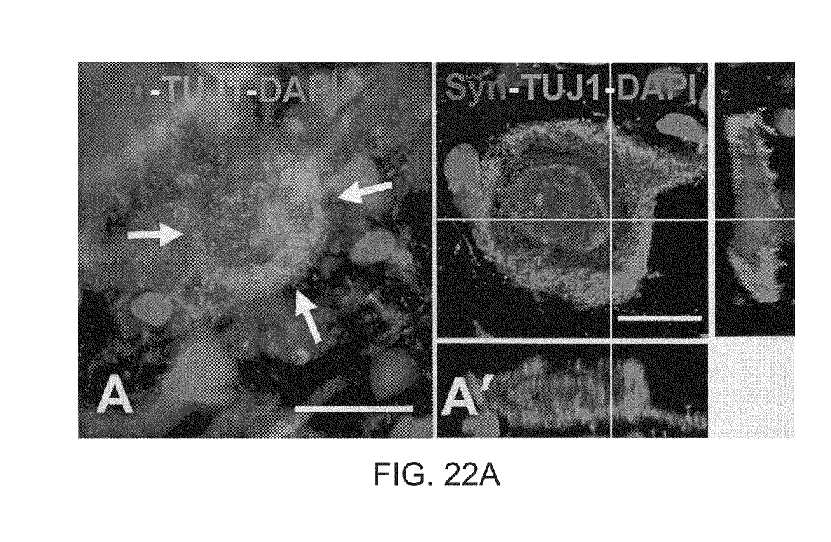

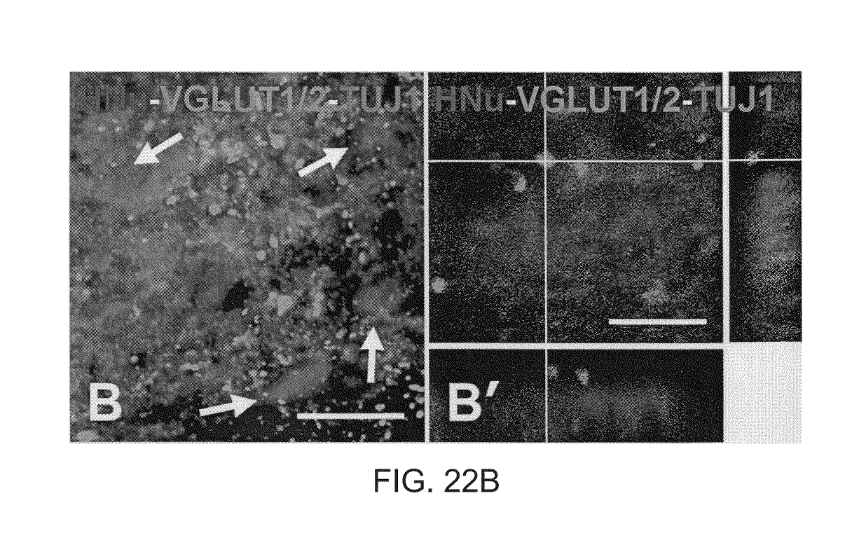

When synaptic terminal markers specifying graft origin were combined with generic neuronal markers, large numbers of human synaptophysin (+) boutons—representing terminals of graft-derived neurons—were found apposed to host neurons, especially surviving α-motor neurons (FIG. 22A). Confocal analysis of specimens from one month post-grafting and end-stage animals shows anatomical patterns typical of synaptic contacts (FIG. 22A′). Conversely, HNu and TUJ1 (+) cells, i.e. graft-derived neurons, were closely apposed to VGLUT1/2 (+) terminals of mouse origin (FIG. 22B); the synaptic significance of such appositions was difficult to determine by confocal microscopy because of the high cell density of grafts (FIG. 22B′). Together, these patterns of reciprocal innervation between host and graft-derived neurons indicate a substantial degree of structural integration of NSC grafts within the host circuitry. This phenomenon is likely to account for the beneficial effects of NSCs on clinical outcomes laid out in the previous section.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

TABLE 1
Primary Antibodies

| Phenotypes | Target proteins/epitopes | Host | Dilution | Vendor |
|---|---|---|---|---|
| Neural stem cell | nestinq | mouse | 1:400 | Chemicon, Temecula, CA |
| | nestin | rabbit | 1:200 | Chemicon |
| Neuronal- | Polysialylated neuronal cell adhesion | mouse | 1:400 | Chemicon |
| Restricted | molecule (PSA-NCAM) | | | |
| Precursor | | | | |
| Human Identity | human nuclear protein antibody (HNu) | mouse | 1:800 | Chemicon |
| Mitotic marker | Ki67, antigen (NCL-Ki67p) | rabbit | 1:1000 | Novocastra Labs, Newcastle, UK |
| Neuron-general | type III β-tubulin epitope J1 (TUJ1) | mouse | 1:1000 | Covance, Berkeley, CA |
| | neuronal nuclear epitopes (NeuN) | mouse | 1:1000 | Chemicon, |
| | doublecortin (Dex, C-18) | goat | 1:400 | Santa Cruz Biotech, Santa Cruz, CA |
| Neuron-human- | 70 kD neurofilament (NF70) | mouse | 1:100 | Chemicon |
| specific | synaptophysin (Syn) | mouse | 1:600 | Chemicon |
| Neuron-rodent | bassoon (Bsn) | mouse | 1:400 | Stressgen, Victoria, BC |
| specific | | | | |
| Neuron-motor | Islet (Islt 1) | mouse | 1:1000 | DSHB, Iowa City, Iowa |
| neuron | HB-9 | mouse | 1:1000 | DSHB, Iowa City, Iowa |
| development- | Low-affinity neurotrophin receptor | rabbit | 1:200 | Promega, Madison, WI |
| related | (p75^{NTR}) | | | |
| Neuro-transmitter | glutamate decarboxylase 65, 67 (GAD) | rabbit | 1:400 | Chemicon |
| enzymes/receptors/ | glutamate decarboxylase 67 (GAD) | mouse | 1:600 | Chemicon |
| transporters | choline acetyltransferase (ChAT) | goat | 1:100 | Chemicon |
| | vesicular glutamate transporter 1 | guinea | 1:2000 | Chemicon |
| | (VGLUT1) | pig | | |
| | vesicular glutamate transporter 2 | guinea | 1:2000 | Chemicon |
| | (VGLUT2) | pig | | |
| | glutamate receptor subunit 2&3 | rabbit | 1:100 | Chemicon |
| | (GluR2/3) | | | |
| Astrocyte | glial fibrillary acidic protein (GFAP) | rabbit | 1:400 | Dako, Carpinteria, CA |
| Ogliodendrocyte | A2B5 | mouse | 1:200 | Chemicon |
| | O4 | mouse | 1:25 | Chemicon |
| | CNPas | mouse | 1:100 | Chemicon |
| | Rip | mouse | 1:100 | Chemicon |

TABLE 2
Primer sequences used for real-time PCR

| | Primer Sequence |
|---|---|
| Neurotrophic | |
| Factor | |
| FGF-2 | sense 5′-GTG TGC TAA CCG TAC CTG GC-3′ |
| | antisense 5′-CTG GTG ATT TCC TTG ACC GG-3′ |
| BDNF | sense 5′-CCA AGG CAG GTT CAA GAG G-3′ |
| | antisense 5′-TCC AGC AGA AAG AGA AGA GGA-3′ |
| VEGF | sense 5′-CCA TGG CAG AAG GAG GAG G-3′ |
| | antisense 5′-ATT GGA TGG CAC TAG CTG CG-3′ |
| GDNF | sense 5′-CTG ACT TGG GTC TGG GCT ATG-3′ |
| | antisense 5′-TTG TCA CTC ACC AGC CTT CTA TTT-3′ |
| IGF-1 | sense 5′-CAA CAA GCC CAC AGG GTA TGG-3′ |
| | antisense 5′-GCA CTC CCT CTA CTT GCG TTC-3′ |
| Neuregulin | |
| NRG1 | sense 5′-CTT GTA AAA TGT GCG GAG A-3′ |
| | antisense 5′-GTC TTT CAC CAT GAA GCA CT-3′ |
| NRG2 | sense 5′-GAG ACA GCC AAG TCC TAT TG-3′ |
| | antisense 5′-CCC TCG ATG TAG TAG CAG AC-3′ |
| NRG3 | sense 5′-AGG ACC TTG CAT ACT GTC TC-3′ |
| | antisense 5′-ACT CCT TGG TAG CCT TCT TT-3′ |
| Standard | |
| 18s rRNA | sense 5′-CGC CGC TAG AGG TGA AAT TC-3′, |
| | antisense 5′-TTG GCA AAT GCT TTC GCT C-3′ |

The following documents are referred to above by reference to the below sequential numbering with such number designations above generally set forth within brackets (i.e. [ ]).
1. Mallet J, Björklund A, Caskey C T, Gage F H, Hefti F, et al. (1991) Group report: neuronal replacement and functional modification. In: Price D L, Thoenen H, Aguayo A J, editors. Neurodegenerative Disorders. Mechanisms and Prospects for Therapy. New York: John Wiley & Sons. pp. 271-290.
2. Dumont A S, Dumont R J, Oskouian R J (2002) Will improved understanding of the pathophysiological mechanisms involved in acute spinal cord injury improve the potential for therapeutic intervention? Curr Opin Neurol 15: 713-720.
3. Gage F H (2000) Mammalian neural stem cells. Science 287: 1433-1438.
4. Lindvall 0, Kokaia Z, Martinez-Serrano A (2004) Stem cell therapy for human neurodegenerative disorders-how to make it work. Nat Med 10 Suppl: S42-S50.
5. Park K I, Ourednik J, Ourednik V, Taylor R M, Aboody-Guterman K S, Auguste K I, Lachyankar M B, Redmond D E, Snyder E Y (2002) Global gene and cell replacement strategies via stem cells. Gene Ther 9: 613-624.
6. Cao Q L, Howard R M, Dennison J B, Whittemore S R (2002) Differentiation of engrafted neuronal-restricted precursor cells is inhibited in the traumatically injured spinal cord. Experimental Neurology 177: 349-359.
7. Yan J, Welsh A M, Bora S H, Snyder E Y, Koliatsos V E (2004) Differentiation and tropic/trophic effects of exogenous neural precursors in the adult spinal cord. J Comp Neurol 480: 101-114.
8. Shihabuddin L S, Homer P J, Ray J, Gage F H (2000) Adult spinal cord stem cells generate neurons after transplantation in the adult dentate gyrus. J Neurosci 20: 8727-8735.
9. Cao Q L, Zhang Y P, Howard R M, Walters W M, Tsoulfas P, Whittemore S R (2001) Pluripotent stem cells engrafted into the normal or lesioned adult rat spinal cord are restricted to a glial lineage. Exp Neurol 167: 48-58.
10. McDonald J W, Liu X Z, Qu Y, Liu S, Mickey S K, Turetsky D, Gottlieb D I, Choi D W (1999) Transplanted embryonic stem cells survive, differentiate and promote recovery in injured rat spinal cord. Nature Med 5: 1410-1412.
11. Han S S W, Kang D Y, Mujtaba T, Rao M S, Fischer I (2002) Grafted lineage-restricted precursors differentiate exclusively into neurons in the adult spinal cord. Experimental Neurology 177: 360-375.
12. Rothstein J D, Martin L J, Kuncl R W. Decreased glutamate transport by the brain and spinal cord in amyotrophic lateral sclerosis. N Engl J Med 1992; 326:1464-1468.
13. Howland D S, Liu J., She Y, Goad B, Maragakis N J, Kim B et al. Focal loss of the glutamate transporter EAAT2 in a transgenic rat model of SOD1 mutant-mediated amyotrophic lateral sclerosis (ALS). Proc Natl Acad Sci USA 2002; 99(3):1604-1609.
14. Turner B J, Atkin J D, Farg M A, Zang d W, Rembach A, Lopes E C et al. Impaired extracellular secretion of mutant superoxide dismutase 1 associates with neurotoxicity in familial amyotrophic lateral sclerosis. J Neurosci 2005; 25(1):108-117.
15. Park K I, Ourednik J, Ourednik V, Taylor R M, Aboody-Guterman K S, Auguste K I et al. Global gene and cell replacement strategies via stem cells. Gene Ther 2002; 9(10):613-624.
16. Cao Q L, Howard R M, Dennison J B, Whittemore S R. Differentiation of engrafted neuronal-restricted precursor cells is inhibited in the traumatically injured spinal cord. Experimental Neurology 2002; 177(2):349-359.
17. Yan J, Welsh A M, Bora S H, Snyder E Y, Koliatsos V E. Differentiation and tropic/trophic effects of exogenous neural precursors in the adult spinal cord. J Comp Neurol 2004; 480(1):101-114.
18. Bruijn L I, Miller T M, Cleveland D W. Unraveling the mechanisms involved in motor neuron degeneration in ALS. ANNU. REV. NEUROSCI 2004; 27:723-749.
19. Price D L, Wong P L, Koliatsos V E et al. Motor Neuron Disease. In: Siegel G J, Alberts R W, Brady S T et al., eds. Basic Neurochemistry: Elsevier Science, 2005.
20. Gurney M E, Pu H, Chiu A Y et al. Motor neuron degeneration in mice that express a human Cu,Zn superoxide dismutase mutation. SCIENCE 1994; 264:1772-1775.
21. Wong P C, Pardo C A, Borchelt D R et al. An adverse property of a familial ALS-linked SOD1 mutation causes motor neuron disease characterized by vacuolar degeneration of mitochondria. NEURON 1995; 14:1105-1116.
22. Cleveland D W, Rothstein J D. From Charcot to Lou Gehrig: deciphering selective motor neuron death in ALS. NAT. REV. NEUROSCI 2001; 2(11):806-819.
23. Mallet J, Bjorklund A, Caskey C T et al. Group report: neuronal replacement and functional modification. In: Price D L, Thoenen H, Aguayo A J, eds. Neurodegenerative Disorders. Mechanisms and Prospects for Therapy. New York: John Wiley & Sons, 1991:271-290.
24. Tai Y T, Svendsen C N. Stem cells as a potential treatment of neurological disorders. CURR. OPIN. PHARMACOL. 2004; 4(1):98-104.
25. Silani V, Cova L, Corbo M et al. Stem-cell therapy for amyotrophic lateral sclerosis. LANCET 2004; 364(9429):200-202.
26. Ostenfeld T, Caldwell M A, Prowse K R et al. Human neural precursor cells express low levels of telomerase in vitro and show diminishing cell proliferation with extensive axonal outgrowth following transplantation. EXP NEUROL 2000; 164(1):215-226.
27. Englund U, Bjorklund A, Wictorin K. Migration patterns and phenotypic differentiation of long-term expanded human neural progenitor cells after transplantation into the adult rat brain. BRAIN RES. DEV. BRAIN RES. 2002; 134(1-2):123-141.
28. Hurelbrink C B, Barker R A. Migration of cells from primary transplants of allo- and xenografted foetal striatal tissue in the adult rat brain. EUR. J. NEUROSCI. 2005; 21(6):1503-1510.
29. Wood M J, Sloan D J, Wood K J et al. Indefinite survival of neural xenografts induced with anti-CD4 monoclonal antibodies. NEUROSCIENCE 1996; 70(3):775-789.
30. Klein S M, Behrstock S, McHugh J et al. GDNF Delivery Using Human Neural Progenitor Cells in a Rat Model of ALS. HUM. GENE THER. 2005; 16(4):509-521.
31. Li P, Tessler A, Han S S et al. Fate of immortalized human neuronal progenitor cells transplanted in rat spinal cord. ARCH. NEUROL 2005; 62(2):223-229.
32. Yan J, Xu L, Welsh A M et al. Grafting of Human Neural Stem Cells (hNSCs) into the Spinal Cord of a Mouse Model of ALS (SOD1-G93A) Delays the Symptoms and Extends Life Span [abstract]. SOC. NEUROSCI. ABS. 2005; 131.3.
33. Xu L, Yan J, Chen D W A M et al. Human neural stem cell (NSC) grafts in the spinal cord of SOD1-G93A transgenic rats delay motor neuron degeneration and prolong life span [abstract]. SOC. FOR NEUROSCI. ABST. 2005:213.3.
34. Koliatsos V E, Price W L, Pardo C A, Price D L (1994) Ventral root avulsion: an experimental model of death of adult motor neurons. J Comp Neurol 342: 35-44.
35. Ikonomidou C, Qin Y Q, Labruyere J, Olney J W (1996) Motor neuron degeneration induced by excitotoxin agonists has features in common with those seen in the SOD-1 transgenic mouse model of amyotrophic lateral sclerosis. J Neuropathol Exp Neurol 55: 211-224.
36. Vescovi A L, Parati E A, Gritti A, Poulin P, Ferrario M, Wanke E, Frolichsthal-Schoeller P, Cova L, Arcellana-Panlilio M, Colombo A, Galli R (1999) Isolation and cloning of multipotential stem cells from the embryonic human CNS and establishment of transplantable human neural stem cell lines by epigenetic stimulation. Exp Neurol 156: 71-83.
37. Gundersen H J G, Bagger P, Bendtsen T F, Evans S M, Korbo L, Marcussen N, Moller A, Nielsen K, Nyengaard J R, Pakkenberg B, Sorensen F B, Vesterby A, West M J (1988) The new stereological tools: disector, fractionator, nucleator and point sampled intercepts and their use in pathological research and diagnosis. APMIS 96: 857-881.
38. Hesling C, D'Incan M, D'Incan C, Souteyrand P, Monboisse J C, Pasco S, Madelmont J C, Bignon Y J (2004) Downregulation of BRCA1 in A375 melanoma cell line increases radio-sensitivity and modifies metastatic and angiogenic gene expression. J Invest Dermatol 122: 369-380.
39. Schulte-Herbruggen o, Nassenstein C, Lommatzsch M, Quarcoo D, Renz H, Braun A (2005) Tumor necrosis factor-alpha and interleukin-6 regulate secretion of brain-derived neurotrophic factor in human monocytes. J Neuroimmunol 160: 204-209.
40. Kozlowski D A, Bremer E, Redmond D E, Jr., George D, Larson B, Bohn M C (2001) Quantitative analysis of transgene protein, mRNA, and vector DNA following injection of an adenoviral vector harboring glial cell line-derived neurotrophic factor into the primate caudate nucleus. Mol Ther 3: 256-261.
41. Wang X, Seed B (2003) A PCR primer bank for quantitative gene expression analysis. Nucleic Acids Res 31: e154.
42. Dunn M, Sinha P, Campbell R, Blackburn E, Levinson N, Rampaul R, Bates T, Humphreys S, Gullick W J (2004) Co-expression of neuregulins 1, 2, 3 and 4 in human breast cancer. J Pathol 203: 672-680.
43. Pfaffl M W (2001) A new mathematical model for relative quantification in real-time RT-PCR. Nucleic Acids Research 29:
44. Johe K K, Hazel T G, Muller T, Dugich-Djordjevic M M, McKay R D. Single factors direct the differentiation of stem cells from the fetal and adult central nervous system. Genes Dev 1996; 10(24):3129-3140.
45. Basso D M, Beattie M S, Bresnahan J C. A sensitive and reliable locomotor rating scale for open field testing in rats. J Neurotrauma 1995; 12(1):1-21.
46. Klein S M, Behrstock S, McHugh J, Hoffmann K, Wallace K, Suzuki M et al. GDNF Delivery Using Human Neural Progenitor Cells in a Rat Model of ALS. Hum Gene Ther 2005; 16(4):509-521.
47. Rivlin A S, Tator C H. Objective clinical assessment of motor function after experimental spinal cord injury in the rat. J Neurosurg 1977; 47(4):577-581.
48. Weydt P, Hong S Y, Kliot M, Moller T. Assessing disease onset and progression in the SOD1 mouse model of ALS. Neuroreport 2003; 14(7):1051-1054.
49. Swett J E, Wikholm R P, Blanks R H, Swett A L, Conley L C. Motoneurons of the rat sciatic nerve. Exp Neurol 1986; 93(1):227-252.
50. Sheng J G, Bora S H, Xu G, Borchelt D R, Price D L, Koliatsos V E. Lipopolysaccharide-induced-neuroinflammation increases intracellular accumulation of amyloid precursor protein and amyloid beta peptide in APPswe transgenic mice. Neurobiology of Disease 2003; 14(1):133-145.
51. Pfaffl M W. A new mathematical model for relative quantification in real-time RT-PCR. Nucleic Acids Research 2001; 29(9).
52. Vandesompele J, De Preter K, Pattyn F, Poppe B, Van Roy N, De Paepe A et al. Accurate normalization of real-time quantitative RT-PCR data by geometric averaging of multiple internal control genes. Genome Biol 2002; 3(7):RESEARCH0034.
53. Yan Q, Elliott J, Snider W D. Brain-derived neurotrophic factor rescues spinal motor neurons from axotomy-induced cell death. Nature 1992; 360:753-755.
54. Yan Q, Matheson C, Lopez O T. In vivo neurotrophic effects of GDNF on neonatal and adult facial motor neurons. Nature 1995; 373:341-344.
55. Henderson C E, Phillips H S, Pollock R A, Davies A M, Lemeulle C, Armanini M P et al. GDNF: a potent survival factor for motoneurons present in peripheral nerve and muscle. Science 1994; 266:1062-1064.
56. Koliatsos V E, Clatterbuck R E, Winslow J W, Cayouette M H, Price D L. Evidence that brain-derived neurotrophic factor is a trophic factor for motor neurons in vivo. Neuron 1993; 10(3):359-367.
57. Van Den B L, Storkebaum E, Vleminckx V, Moons L, Vanopdenbosch L, Scheveneels W et al. Effects of vascular endothelial growth factor (VEGF) on motor neuron degeneration. Neurobiol Dis 2004; 17(1):21-28.
58. Hellstrom J, Oliveira A L R, Meister B, Cullheim S. Large cholinergic nerve terminals on subsets of motoneurons and their relation to muscarinic receptor type 2. Journal of Comparative Neurology 2003; 460(4):476-486.
59. Rind H B, Butowt R, von Bartheld C S. Synaptic targeting of retrogradely transported trophic factors in motoneurons: Comparison of glial cell line-derived neurotrophic factor, brain-derived neurotrophic factor, and cardiotrophin-1 with tetanus toxin. Journal of Neuroscience 2005; 25(3):539-549.
60. Johe K K, Hazel T G, Muller T et al. Single factors direct the differentiation of stem cells from the fetal and adult central nervous system. GENES AND DEVELOPMENT 1996; 10(24):3129-3140.
61. Weydt P, Hong S Y, Kliot M et al. Assessing disease onset and progression in the SOD1 mouse model of ALS. NEUROREPORT 2003; 14(7):1051-1054.
62. Basso D M, Beattie M S, Bresnahan J C. A sensitive and reliable locomotor rating scale for open field testing in rats. J NEUROTRAUMA 1995; 12(1):1-21.
63. Wrathall J R, Pettegrew R K, Harvey F. Spinal cord contusion in the rat: production of graded, reproducible, injury groups. EXP. NEUROL 1985; 88(1):108-122.
64. Vescovi A L, Parati E A, Gritti A et al. Isolation and cloning of multipotential stem cells from the embryonic human CNS and establishment of transplantable human neural stem cell lines by epigenetic stimulation. EXP. NEUROL 1999; 156(1):71-83.
65. Ortaldo J R, Winkler-Pickett R, Mason A T et al. The Ly-49 family: regulation of cytotoxicity and cytokine production in murine CD3+ cells. J. IMMUNOL. 1998; 160(3):1158-1165.
66. Imai Y, Ibata I, Ito D et al. A novel gene iba1 in the major histocompatibility complex class III region encoding an EF hand protein expressed in a monocytic lineage. BIOCHEM. BIOPHYS. RES. COMMUN. 1996; 224(3):855-862.
67. Ito D, Imai Y, Ohsawa K et al. Microglia-specific localisation of a novel calcium binding protein, Iba1. BRAIN RES. MOL. BRAIN RES. 1998; 57(1):1-9.
68. Koliatsos V E, Price W L, Pardo C A et al. Ventral root avulsion: an experimental model of death of adult motor neurons. J COMP NEUROL 1994; 342(1):35-44.
69. Enomoto M, Shinomiya K, Okabe S (2003) Migration and differentiation of neural progenitor cells from two different regions of embryonic central nervous system after transplantation into the intact spinal cord. European Journal of Neuroscience 17: 1223-1232.
70. Horner P J, Power A E, Kempermann G, Kuhn H G, Palmer T D, Winkler J, Thal L J, Gage F H (2000) Proliferation and differentiation of progenitor cells throughout the intact adult rat spinal cord. J Neurosci 20: 2218-2228.
71. Iwanami A, Kaneko S, Nakamura M, Kanemura Y, Mori H, Kobayashi S, Yamasaki M, Momoshima S, Ishii H, Ando K, Tamaoka Y, Tamaoki N, Nomura T, Toyama Y, Okano H (2005) Transplantation of human neural stem cells for spinal cord injury in primates. J Neurosci Res 80: 182-190.
72. Xu L, Yan J, Chen D W A M, Hazel T, Johe K, Koliatsos V E (2005) Human neural stem cell (NSC) grafts in the spinal cord of SOD1-G93A transgenic rats delay motor neuron degeneration and prolong life span. Soc for Neurosci Abst 213.3.
73. Yan J, Xu L, Welsh A M, Chen D H T, Johe K, Koliatsos V E (2005) Grafting of Human Neural Stem Cells (hNSCs) into the Spinal Cord of a Mouse Model of ALS (SOD1-G93A) Delays the Symptoms and Extends Life Span. Soc Neurosci Abs 131.3:
74. Cummings B J, Uchida N, Tamaki S J, Salazar D L, Hooshmand M, Summers R, Gage F H, Anderson A J (2005) Human neural stem cells differentiate and promote locomotor recovery in spinal cord-injured mice. Proc Natl Acad Sci USA 102: 14069-14074.
75. Schneider S P, Fyffe R E (1992) Involvement of GABA and glycine in recurrent inhibition of spinal motoneurons. J Neurophysiol 68: 397-406.
76. Malcangio M, Bowery N G (1996) GABA and its receptors in the spinal cord. Trends Pharmacol Sci 17: 457-462.
77. Ma W, Behar T, Chang L, Barker J L (1994) Transient increase in expression of GAD65 and GAD67 mRNAs during postnatal development of rat spinal cord. J Comp Neurol 346: 151-160.
78. Houenou L J, Haverkamp L J, McManaman J L, Oppenheim R W (1991) The Regulation of Motoneuron Survival and Differentiation by Putative Muscle-Derived Neurotrophic Agents—Neuromuscular Activity and Innervation. Development 149-155.
79. Yin Q W, Oppenheim R W (1992) Modifications of Motoneuron Development Following Transplantation of Thoracic Spinal-Cord to the Lumbar Region in the Chick-Embryo—Evidence for Target-Derived Signals That Regulate Differentiation. Journal of Neurobiology 23: 376-395.
80. Wu P, Tarasenko Y I, Gu Y, Huang L Y, Coggeshall R E, Yu Y (2002) Region-specific generation of cholinergic neurons from fetal human neural stem cells grafted in adult rat. Nat Neurosci 5: 1271-1278.
81. Gao J, Coggeshall R E, Tarasenko Y I, Wu P (2005) Human neural stem cell-derived cholinergic neurons innervate muscle in motoneuron deficient adult rats. Neuroscience 131: 257-262.
82. Filbin M T (2003) Myelin-associated inhibitors of axonal regeneration in the adult mammalian CNS. Nat Rev Neurosci 4: 703-713.
83. Spencer T, Filbin M T (2004) A role for cAMP in regeneration of the adult mammalian CNS. J Anat 204: 49-55.
84. Harper J M, Krishnan C, Darman J S, Deshpande D M, Peck S, Shats I, Backovic S, Rothstein J D, Kerr D A (2004) Axonal growth of embryonic stem cell-derived motoneurons in vitro and in motoneuron-injured adult rats. Proc Natl Acad Sci USA 101: 7123-7128.
85. Henderson C E, Phillips H S, Pollock R A, Davies A M, Lemeulle C, Armanini M P, Simpson L C, Moffet B, Vandlen R A, Koliatsos V E, Rosenthal A (1994) GDNF: a potent survival factor for motoneurons present in peripheral nerve and muscle. Science 266: 1062-1064.
86. Beck K D, Valverde J, Alexi T, Poulsen K, Moffat B, Vandlen R A, Rosenthal A, Hefti F (1995) Mesencephalic dopaminergic neurons protected by GDNF from axotomy-induced degeneration in the adult brain. Nature 373: 339-341.
87. Koliatsos V E, Clatterbuck R E, Winslow J W, Cayouette M H, Price D L (1993) Evidence that brain-derived neurotrophic factor is a trophic factor for motor neurons in vivo. Neuron 10: 359-367.
88. Meyer D, Birchmeier C (1996) Multiple essential functions of neuregulin in development. Nature 378: 386-390.
89. Grinspan J B, Marchionni M A, Reeves M, Coulaloglou M, Scherer S S (1996) Axonal interactions regulate Schwalm cell apoptosis in developing peripheral nerve: neuregulin receptors and the role of neuregulins. J Neurosci 16: 6107-6118.
90. Kopp D M, Trachtenberg J T, Thompson W J (1997) Glial growth factor rescues Schwann cells of mechanoreceptors from denervation-induced apoptosis. J Neurosci 17: 6697-6706.
91. Parkinson D B, Dong Z, Bunting H, Whitfield J, Meier C, Marie H, Mirsky R, Jessen K R (2001) Transforming growth factor beta (TGFbeta) mediates Schwann cell death in vitro and in vivo: examination of c-Jun activation, interactions with survival signals, and the relationship of TGFbeta-mediated death to Schwann cell differentiation. J Neurosci 21: 8572-8585.
92. Fainzilber M, Smit A B, Syed N I, Wildering W C, Hermann, van der Schors R C, Jimenez C, Li K W, van Minnen J, Bulloch A G, Ibanez C F, Geraerts W P (1996) CRNF, a molluscan neurotrophic factor that interacts with the p75 neurotrophin receptor. Science 274: 1540-1543.
93. Riethmacher D, Sonnenberg-Riethmacher E, Brinkmann V, Yamaai T, Lewin G R, Birchmeier C (1997) Severe neuropathies in mice with targeted mutations in the ErbB3 receptor. Nature 389: 725-730.
94. Oka N, Kawasaki T, Matsui M, Tachibana H, Sugita M, Akiguchi I (2000) Neuregulin is associated with nerve regeneration in axonal neuropathies. Neuroreport 11: 3673-3676.
95. Azzouz M, Ralph G S, Storkebaum E, Walmsley L E, Mitrophanous K A, Kingsman S M, Carmeliet P, Mazarakis N D (2004) VEGF delivery with retrogradely transported lentivector prolongs survival in a mouse ALS model. Nature 429: 413-417.
96. Zheng C, Nennesmo I, Fadeel B, Henter J I (2004) Vascular endothelial growth factor prolongs survival in a transgenic mouse model of ALS. Ann Neurol 56: 564-567.
97. Storkebaum E, Lambrechts D, Dewerchin M, Moreno-Murciano M P, Appelmans S, Oh H, Van Damme P, Rutten B, Man W Y, De Mol M, Wyns S, Manka D, Vermeulen K, Van Den B L, Mertens N, Schmitz C, Robberecht W, Conway E M, Collen D, Moons L, Carmeliet P (2005) Treatment of motoneuron degeneration by intracerebroventricular delivery of VEGF in a rat model of ALS. Nat Neurosci 8: 85-92.
98. Schwarz Q, Gu C, Fujisawa H, Sabelko K, Gertsenstein M, Nagy A, Taniguchi M, Kolodkin A L, Ginty D D, Shima D T, Ruhrberg C (2004) Vascular endothelial growth factor controls neuronal migration and cooperates with Sema3A to pattern distinct compartments of the facial nerve. Genes Dev 18: 2822-2834.
99. Arsenijevic Y, Weiss S (1998) Insulin-like growth factor-I is a differentiation factor for postmitotic CNS stem cell-derived neuronal precursors: distinct actions from those of brain-derived neurotrophic factor. J Neurosci 18: 2118-2128.
100. Brevig T, Holgersson J, Widner H. Xenotransplantation for CNS repair: immunological barriers and strategies to overcome them. TRENDS 1N NEUROSCIENCE 2000; 23(8):337-344.
101. Larsson L C, Anderson P, Widner H et al. Enhanced survival of porcine neural xenografts in mice lacking CD1d1, but no effect of NK1.1 depletion. CELL TRANSPLANT. 2001; 10(3):295-304.
102. Masri M A. The mosaic of immunosuppressive drugs. MOL. IMMUNOL. 2003; 39(17-18):1073-1077.
103. Larsson L C, Frielingsdorf H, Mirza B et al. Porcine neural xenografts in rats and mice: donor tissue development and characteristics of rejection. EXP. NEUROL. 2001; 172(1):100-114.
104. Larsson L C, Czech K A, Widner H et al. Discordant neural tissue xenografts survive longer in immunoglobulin deficient mice. TRANSPLANTATION 1999; 68(8):1153-1160.
105. Barker R A, Ratcliffe E, McLaughlin M et al. A role for complement in the rejection of porcine ventral mesencephalic xenografts in a rat model of Parkinson's disease. J. NEUROSCI. 2000; 20(9):3415-3424.
106. Barker R A, Widner H. Immune problems in central nervous system cell therapy. NEURORX. 2004; 1(4):472-481.
107. Hurelbrink C B, Armstrong R J, Dunnett S B et al. Neural cells from primary human striatal xenografts migrate extensively in the adult rat CNS. EUR. J. NEUROSCI. 2002; 15(7):1255-1266.
108. Yan J, Welsh A M, Hazel T et al. Grafted human neural stem (NS) cells differentiate into neurons, migrate long distance and project axons in spinal cord and the roots of adult rats. [abstract]. SOClETY FOR NEUROSCIENCE ABSTRACTS 2003; 29(150.19).
109. Qi S, Xu D, Peng J et al. Effect of tacrolimus (FK506) and sirolimus (rapamycin) mono- and combination therapy in prolongation of renal allograft survival in the monkey. TRANSPLANTATION 2000; 69(7):1275-1283.
110. Kaminska B, Gaweda-Walerych K, Zawadzka M. Molecular mechanisms of neuroprotective action of immunosuppressants—facts and hypotheses. J CELL MOL. MED. 2004; 8(1):45-58.
111. Klettner A, Herdegen T. FK506 and its analogs—therapeutic potential for neurological disorders. CURR. DRUG TARGETS. CNS. NEUROL DISORD. 2003; 2(3):153-162.
112. Karlsson J, Fong K S, Hansson M J et al. Life span extension and reduced neuronal death after weekly intraventricular cyclosporin injections in the G93A transgenic mouse model of amyotrophic lateral sclerosis. J NEUROSURG. 2004; 101(1):128-137.
113. Anneser J M, Gmerek A, Gerkrath J et al. Immunosuppressant FK506 does not exert beneficial effects in symptomatic G93A superoxide dismutase-1 transgenic mice. NEUROREPORT 2001; 12(12):2663-2665.
114. Hottinger A F, Fine E G, Gurney M E, Zurn A D, Aebischer P. The copper chelator d-penicillamine delays onset of disease and extends survival in a transgenic mouse model of familial amyotrophic lateral sclerosis. Eur J Neurosci 1997; 9:1548-1551.
115. Kriz J, Nguyen M D, Julien J P. Minocycline slows disease progression in a mouse model of amyotrophic lateral sclerosis. Neurobiol Dis 2002; 10(3):268-278.
116. Kriz J, Gowing G, Julien J P. Efficient three-drug cocktail for disease induced by mutant superoxide dismutase. Ann Neurol 2003; 53(4):429-436.
117. Klivenyi P, Kiaei M, Gardian G, Calingasan N Y, Beal M F. Additive neuroprotective effects of creatine and cyclooxygenase 2 inhibitors in a transgenic mouse model of amyotrophic lateral sclerosis. J Neurochem 2004; 88(3):576-582.
118. Kiaei M, Kipiani K, Petri S, Choi D K, Chen J, Calingasan N Y et al. Integrative role of cPLA with COX-2 and the effect of non-steriodal anti-inflammatory drugs in a transgenic mouse model of amyotrophic lateral sclerosis. J Neurochem 2005; 93(2):403-411.
119. Acsadi G, Anguelov R A, Yang H, Toth G, Thomas R, Jani A et al. Increased survival and function of SOD1 mice after glial cell-derived neurotrophic factor gene therapy. Hum Gene Ther 2002; 13(9):1047-1059.
120. Wang L J, Lu Y Y, Muramatsu S, Ikeguchi K, Fujimoto K, Okada T et al. Neuroprotective effects of glial cell line-derived neurotrophic factor mediated by an adeno-associated virus vector in a transgenic animal model of amyotrophic lateral sclerosis. J Neurosci 2002; 22(16):6920-6928.
121. Guillot S, Azzouz M, Deglon N, Zurn A, Aebischer P. Local GDNF expression mediated by lentiviral vector protects facial nerve motoneurons but not spinal motoneurons in SOD1(G93A) transgenic mice. Neurobiol Dis 2004; 16(1):139-149.
122. Azzouz M, Ralph G S, Storkebaum E, Walmsley L E, Mitrophanous K A, Kingsman S M et al. VEGF delivery with retrogradely transported lentivector prolongs survival in a mouse ALS model. Nature 2004; 429(6990):413-417.
123. Zheng C, Nennesmo I, Fadeel B, Henter J I. Vascular endothelial growth factor prolongs survival in a transgenic mouse model of ALS. Ann Neurol 2004; 56(4):564-567.
124. Storkebaum E, Lambrechts D, Dewerchin M, Moreno-Murciano M P, Appelmans S, Oh H et al. Treatment of motoneuron degeneration by intracerebroventricular delivery of VEGF in a rat model of ALS. Nat Neurosci 2005; 8(1):85-92.
125. Gurney M E, Pu H, Chiu A Y, Dal Canto M C, Polchow C Y, Alexander D D et al. Motor neuron degeneration in mice that express a human Cu,Zn superoxide dismutase mutation. Science 1994; 264:1772-1775.
126. Wong P C, Pardo C A, Borchelt D R, Lee M K, Copeland N G, Jenkins N A et al. An adverse property of a familial ALS-linked SOD1 mutation causes motor neuron disease characterized by vacuolar degeneration of mitochondria. Neuron 1995; 14:1105-1116.
127. Cummings B J, Uchida N, Tamaki S J, Salazar D L, Hooshmand M, Summers R et al. Human neural stem cells differentiate and promote locomotor recovery in spinal cord-injured mice. Proc Natl Acad Sci USA 2005; 102(39):14069-14074.
128. Llado J, Haenggeli C, Maragakis N J, Snyder E Y, Rothstein J D. Neural stem cells protect against glutamate-induced excitotoxicity and promote survival of injured motor neurons through the secretion of neurotrophic factors. Mol Cell Neurosci 2004; 27(3):322-331.

1-17. (canceled)

18. A method of treating a spinal cord disease or injury in a subject in need thereof comprising:
(a) isolating at least one neural stem cell from a spinal cord of a mammal, wherein the isolated neural stem cell is multipotential;
(b) expanding in vitro said neural stem cell in a dispersed adherent culture to form an expanded population; and
(c) engrafting the expanded population into the spinal cord of said subject.

19. The method of claim 18, wherein the neural stem cell is isolated from a region of a spinal cord of a mammal corresponding to the injured area of the subject.

20. The method of claim 18, wherein the expanded population comprises cells capable of providing trophic factors.

21. The method of claim 18, wherein the method further involves identifying the subject as having a spinal cord disease or injury.

22. The method of claim 18, wherein the expanded population comprises one or more of a neural stem cell, neural precursor cell, or neural progenitor cell.

23. The method of claim 18, wherein the expanded population is injected into the spinal cord of the subject.

24. The method of claim 18, wherein the spinal cord disease is amyotrophic lateral sclerosis (ALS).

25. The method of claim 21, wherein the engrafted population forms neurons capable of producing an inhibitory neurotransmitter.

26. The method of claim 21, wherein the neurons are capable of producing gamma amino butyric acid.

27. A method of treating a spinal cord injury or condition in a subject in need thereof comprising:
(a) isolating at least one human neural stem cell from a spinal cord of a mammal;
(b) expanding in vitro said neural stem cell in a dispersed adherent culture to form an expanded population; and
(c) contacting the spinal cord of the subject with the expanded population of cells, to treat the spinal cord injury or condition.

28. A method of treating amyotrophic lateral sclerosis in an individual in need thereof the method comprising:
(a) isolating at least one human neural stem cell from a spinal cord of a mammal;
(b) expanding in vitro said neural stem cell in a dispersed adherent culture to form an expanded population; and
(c) contacting the spinal cord of the subject with the expanded population to treat the amyotrophic lateral sclerosis.

29. The method of claim 28, wherein the transplanted population secretes one or more trophic factor selected from the group consisting of GDNF, BDNF, pleiotrophin, VEGF, erythropoetin, midkine, and SOD I.
